SEQUENCE LISTING (91 sequences)

SEQ ID NO: 1 - DNA, 102 bp, synthetic construct
  source 1..102: mol_type = other DNA
gagaataaactagtattcttctggtccccacagactcagagagaacccgccaccatgttcgtgttcctggtgctgctgcctctggtgtccagccagtgtgtg

SEQ ID NO: 2 - DNA, 302 bp, synthetic construct
  source 1..302: mol_type = other DNA
tgatgactcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagca

SEQ ID NO: 3 - protein, 508 aa, synthetic construct
ADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDS

SEQ ID NO: 4 - DNA, 1524 bp, synthetic construct
  source 1..1524: mol_type = other DNA
gctgacaccctgtgcattggctatcatgccaacaactccacagacaccgtggacacagtgctggagaagaacgtgacagtgacccactccgtgaacctgctggaagacaagcacaacggcaaactgtgcaagctgagaggcgtggcccctctgcacctgggcaagtgcaacatcgccggctggatcctgggcaacccagagtgcgagagcctgtccacagcccggtcctggtcttacattgtggagacatccaacagcgacaacggcacctgctaccctggcgacttcatcaattacgaggagctgagagagcagctgagctccgtgtccagcttcgagaggttcgagatctttcccaagacaagctcctggcccaaccacgacagcgacaacggggtgacagccgcctgccctcacgcaggcgccaagagcttctacaaaaacctgatctggctcgtgaagaaaggaaagagctaccccaagatcaaccagacctacatcaacgacaagggcaaggaggtgctggtgctttggggcatccaccaccctcccacaattgccgaccagcagtccctgtaccagaacgctgacgcctatgtcttcgtgggcacctctagatacagcaagaagttcaagcctgagatcgccacaagacccaaggtgcgggatcaggagggccggatgaattactactggaccctggtggagccaggcgacaagatcaccttcgaggccaccggcaacctggtggctccccgatacgccttcaccatggagcgggacgccggcagcggcatcatcatcagcgacacacctgtgcacgactgcaacaccacctgccagacccctgaaggagccatcaacaccagcctgcccttccagaatgtccaccccatcaccatcggcaaatgtccaaagtacgtgaagtccaccaaactgagactggccaccggcctgagaaacgtcccctcaatccagagcagaggcctgtttggggccattgccggcttcatcgagggcggatggaccggcatggtcgacggatggtacggatatcaccaccagaacgaacagggaagcggctacgccgccgacctgaagtccacacagaacgccatcgacaagatcaccaacaaggtgaacagcgtgatcgagaaaatgaacacacagttcacagccgtgggcaaggaattcaatcacctggagaagaggatcgagaatctgaacaagaaggtggacgacggcttcctggacatctggacatacaacgcagagctcctggtgctgctggaaaatgagcggacactggactaccacgactccaacgtgaagaacctgtacgagaaggtgcggaatcaactgaagaacaacgccaaggagattggcaacggctgctttgagttctaccacaagtgcgacaacacctgtatggagtccgtgaagaacggcacatatgactaccccaagtacagcgaagaagccaagctgaacagagagaagattgatggagtgaaactggacagc

SEQ ID NO: 5 - protein, 508 aa, synthetic construct
ADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLES

SEQ ID NO: 6 - DNA, 1524 bp, synthetic construct
  source 1..1524: mol_type = other DNA
gctgacaccctgtgcattggctatcatgccaacaacagcaccgacacagtggacacagtgctggaaaagaacgtgaccgtgacacactccgtgaacctgctggaggacaagcacaacggcaaactgtgcaagctgagaggcgtggcccctctgcacctgggcaagtgcaacatcgccggctggatcctgggcaacccagagtgcgagagcctgagcaccgccagctcctggtcttacatcgtggagacacccagctccgacaacggcacatgctaccctggcgacttcatcgactacgaggaactgagagagcagctgagctccgtgtcctctttcgagaggttcgagatcttccccaagacctcatcttggcccaaccacgacagcaacaagggcgtgaccgccgcctgtcctcacgctggcgcaaagagcttctacaagaacctgatctggctcgtgaagaagggaaacagctaccccaagctgagcaagagctacatcaatgacaaaggcaaggaggtgctggtgctatggggcatccatcaccccagcacaagcgctgaccagcagagcctgtatcagaacgctgacgcctacgtcttcgtcggcagctcccggtacagcaagaaattcaagcccgagatcgccatccggcccaaggtgcgggaccaagagggcagaatgaactactactggaccctggtggagcctggcgacaagatcacctttgaggccacaggcaatctggtggtgcccagatacgcctttgccatggagagaaatgccggctccggcatcatcatcagcgacacacctgtgcacgactgcaacaccacctgtcagacacccaagggcgccatcaacacaagcctgccttttcagaacattcaccccatcaccattggcaagtgccccaagtacgtcaagagcaccaaactgcggctggccacaggactgcggaacatccccagcattcagagccggggcctgtttggagccatcgctggcttcatcgagggcggctggaccggcatggtggacggctggtacggctaccatcaccagaatgaacagggcagcggctacgccgctgacctgaagtccacccagaacgccatcgacgaaatcaccaacaaggtcaactctgtgatcgaaaagatgaacacccagttcaccgccgtgggaaaagagttcaaccacctggagaagaggatcgaaaatctgaacaaaaaggtggacgacggctttctggacatctggacctacaacgcagagctcctggtgctgctggaaaatgaaagaacactggactaccacgactccaacgtcaagaacctgtacgagaaggtgcggagccagctgaagaacaacgccaaggagatcggcaacggctgcttcgagttctaccacaagtgcgacaacacctgtatggagtccgtgaagaacggcacatacgactaccccaagtactctgaagaggccaagctgaacagagaggagattgatggagtgaagctggagtcc

SEQ ID NO: 7 - protein, 507 aa, synthetic construct
ADTICIGYHANNSTDTVDTVLEKNVTVTHSVNLLEDSHNGKLCRLKGKAPLQLGNCNIAGWVLGNPECESLLSNRSWSYIAETPNSENGTCYPGDFADYEELREQLSSVSSFERFEIFPKERSWPNHTTRGVTAACPHARKSSFYKNLVWLTEANGSYPNLSRSYVNNQEKEVLVLWGVHHPSNIEEQRALYRKDNAYVSVVSSNYNRRFTPEIAKRPKVRDQSGRMNYYWTLLEPGDTIIFEATGNLIAPWYAFALSRGPGSGIITSNAPLDECDTKCQTPQGAINSSLPFQNIHPVTIGECPKYVRSTKLRMVTGLRNIPSVQSRGLFGAIAGFIEGGWTGMMDGWYGYHHQNEQGSGYAADQKSTQNAINGITNKVNSVIEKMNTQFTAVGKEFNKLEKRMENLNKKVDDGFMDIWTYNAELLVLLENERTLDFHDSNVKNLYEKVKNQLRNNAKELGNGCFEFYHKCDNECMESVKNGTYDYPKYSEESKLNREKIDGVKLES

SEQ ID NO: 8 - DNA, 1521 bp, synthetic construct
  source 1..1521: mol_type = other DNA
gccgacaccatctgcattggataccatgccaacaacagcaccgacaccgtggacacagtgctggaaaagaacgtgaccgtgacccacagcgtgaacctgctggaagacagccacaacggcaaactgtgcagactgaagggcaaggcccccctgcagctgggaaactgcaacatcgccggatgggtgctgggaaatcccgagtgcgagagcctgctgagcaacaggagctggagctacatcgctgaaacccccaacagcgagaacggcacctgctatcctggcgacttcgctgactacgaggaactgcgggagcagctgtcctccgtgtcctcctttgaaagatttgagatcttccccaaggagcggagctggcccaaccacacaaccaggggcgtgaccgccgcctgccctcatgccagaaagagcagcttctacaagaatctggtctggctgaccgaggccaatggcagctatcccaacctgtccagaagctacgtgaacaaccaggagaaggaggtgctggtcctgtggggggtgcaccaccccagcaacatcgaggagcagagagccctgtacagaaaggacaacgcctatgtcagcgtggtgtccagcaactacaacagacggttcacccccgaaattgccaagcggcccaaggtgcgggatcagagcggccggatgaactactactggaccctgctggagcctggagacaccatcatcttcgaggccaccggcaacctgatcgccccttggtacgccttcgccctgagcagaggccctggcagcggcatcatcaccagcaatgcccccctggacgagtgcgacacaaagtgtcagaccccccagggcgccatcaacagctccctgccctttcagaacatccaccctgtgacaatcggggagtgccccaagtacgtgagaagcaccaagctgcggatggtgaccggcctgcggaacattccctctgtgcagtcccggggcctgtttggggccatcgccggcttcatcgagggcggctggaccggcatgatggacggctggtacggctaccaccaccagaacgaacaaggctccggctacgccgctgaccaaaagtccacccagaatgcaatcaacggcatcaccaacaaggtgaattccgtgatcgagaagatgaacacccagttcacagccgtgggcaaggagttcaacaagctggaaaagcggatggagaacctgaacaagaaagtggacgacggcttcatggacatctggacctacaacgctgaactgctggtgctgctggaaaatgaaagaacactggacttccacgactccaacgtgaagaacctgtatgagaaggtgaagaatcagctcagaaacaacgccaaagagctgggcaacggctgcttcgagttctaccacaagtgcgacaacgagtgtatggagtccgtgaagaacggcacatacgactaccccaagtacagcgaggagagcaagctgaacagggagaagatcgatggcgtgaagctggagtcc

SEQ ID NO: 9 - protein, 507 aa, synthetic construct
ADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSFERFEIFPKESSWPNHTVTGVSASCSHNGESSFYRNLLWLTGKNGLYPNLSKSYANNKEKEVLVLWGVHHPPNIGNQKALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLES

SEQ ID NO: 10 - DNA, 1521 bp, synthetic construct
  source 1..1521: mol_type = other DNA
gccgacaccatctgcattggctatcatgccaacaattccacagacaccgtggacacagtgctggagaagaacgtgaccgtgacccactctgtgaacctgctggagaactctcacaacggcaaactgtgcctgctgaagggcatcgcccctctgcagctcggcaactgcagcgtggccggctggatcctgggcaacccagagtgcgaactgctgatctccaaggaaagctggagctacatcgtggagaagcccaaccccgagaacggcacctgttaccctggccactttgccgactacgaggagctgcgggaacagctgtcttccgtgtcctccttcgaaagattcgagatctttcccaaggagagctcctggcccaaccacacagtgacaggcgtctccgccagctgctcccacaatggcgagtcctctttctacagaaacctgctgtggctgaccggcaagaacggcctgtatcccaacctgagcaagagctacgccaacaacaaggagaaggaagtgctggtgctgtggggggtgcaccaccctccgaacatcggcaatcagaaggccctgtatcacacagaaaacgcctacgtcagcgtggtgtcctcccactacagcagaaagttcacccctgagatcgccaagcggcccaaagtgcgggatcaggagggccggatcaattactactggaccctgctggaaccaggcgacacaatcatctttgaggccaacggcaacctgatcgccccaagatacgccttcgccctgagcagaggcttcggctctggcatcatcaacagcaatgccccgatggacaagtgcgatgccaagtgccagaccccacagggcgccatcaacagcagcctgcctttccagaatgtccaccccgtgacaattggcgagtgtcccaagtacgtgagaagcgccaagctgcggatggtgaccggcctgcggaacatcccttccattcagagcagaggcctgtttggcgccatcgccggattcattgaaggcggctggaccggcatggtggacggctggtacggctaccaccaccagaacgaacagggctctggctacgccgctgaccaaaagtccacccagaatgccatcaacggcatcaccaacaaagtcaacagcgtgatcgagaagatgaacacccagttcaccgctgtgggcaaagagttcaacaagctggagcggcggatggaaaacctgaacaaaaaggtggacgacggcttcatcgacatttggacatacaacgcagagctgctggtgctgctggaaaatgagagaaccctggatttccacgacagcaacgtgaagaacctgtacgagaaggtcaaatcccagctgaaaaacaacgccaaggagatcggcaatggctgcttcgagttctaccacaagtgcaacgatgagtgtatggagtccgtgaaaaacggcacctacgactacccgaagtacagcgaggaaagcaagctcaatcgggagaagatcgacggagtgaagctggagagc

SEQ ID NO: 11 - protein, 507 aa, synthetic construct
ADTICIGYHANNSTDTVDTVLEKNVTVTHSVNLLEDSHNGKLCLLKGIAPLQLGNCSVAGWILGNPECESLISKESWSYIVETPNPENGTCYPGYFADYEELREQLSSVSSFERFEIFPKESSWPNHTVTGVTASCSHNGKSSFYRNLLWLTEKNGLYPNLSNSYVNNKEKEVLVLWGVHHPSNIRDQRAIYHTENAYVSVVSSHYSRRFTPEIAKRPKVRGQEGRINYYWTLLEPGDTIIFEANGNLIAPWYAFALSRGFGSGIITSNAPMNECDAKCQTPQGAINSSLPFQNVHPVTIGECPKYVRSTKLRMVTGLRNIPSIQSRGLFGAIAGFIEGGWTGMMDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLES

SEQ ID NO: 12 - DNA, 1521 bp, synthetic construct
  source 1..1521: mol_type = other DNA
gccgacaccatctgcattggctaccatgccaacaacagcaccgacaccgtggacacagtgctggagaagaacgtgacagtgacacactctgtgaacctgctggaggactcccacaacggaaagctgtgcctgctgaagggaatcgcccctctgcagctgggcaactgttccgtcgccggctggatcctgggcaatcctgagtgcgagagcctgatctccaaagaatcttggtcctacatcgtggagacacccaacccagagaacggcacctgctaccccggatactttgccgactacgaggagctgcgggagcaactgtcctcagtgagcagcttcgaaagattcgagatcttccccaaagagagctcttggcccaaccacacagtgacaggcgtgacagcctcctgcagccacaacggaaagagcagcttctacagaaatctgctgtggctcaccgagaagaatggcctgtatcccaacctctccaacagctacgtgaacaacaaggagaaggaagtgctggtgctatggggggtgcaccaccccagcaacatccgggaccagcgggccatctaccacacagagaacgcctacgtcagcgtggtgtcctcccactactctcggagattcactcccgagatcgccaagcggcccaaggtgcggggccaggagggccggatcaattactactggaccctgctggaaccaggcgacaccatcatcttcgaggccaacggcaatctcatcgccccctggtacgctttcgccctgtctcggggcttcggctccggcatcatcacaagcaacgcccccatgaatgagtgcgatgccaagtgccagacaccccagggcgccatcaacagctccctgcccttccagaacgtccaccctgtgaccatcggagagtgtcccaagtacgtgagatccaccaagctgagaatggtgaccggcctgagaaacattcccagcatccagagcagaggcctgttcggagccatcgccggattcattgagggaggatggaccggcatgatggacggctggtacggctaccaccaccagaatgagcagggaagcggctacgccgctgaccagaaatccacacagaacgccatcaacggcatcaccaacaaggtgaacagcgtgatcgaaaagatgaacacccagttcaccgccgtgggcaaagagttcaacaagctggaaagaagaatggagaacctgaacaagaaagtggacgacggcttcctggacatctggacatacaacgcagagctgctggtgctgctggaaaatgagagaacactggacttccacgacagcaacgtcaaaaacctgtacgagaaggtgaagtcacagctgaaaaacaacgccaaggagatcggcaatggctgcttcgaattctaccacaagtgcaacaacgagtgtatggaatccgtgaagaacggcacctacgactacccgaagtactctgaggagagcaaactgaacagagagaaaatcgacggggtgaagctggagagc

SEQ ID NO: 13 - protein, 508 aa, synthetic construct
ADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNKGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNIPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLES

SEQ ID NO: 14 - DNA, 1524 bp, synthetic construct
  source 1..1524: mol_type = other DNA
gctgacaccatctgcattggctaccatgccaacaactccaccgacaccgtggacacagtgctggagaagaacgtgaccgtgacacattccgtgaacctgctggaggattctcacaacggcaagctctgtcggctgaagggcatcgcccctctgcagctgggcaagtgcaacatcgccggctggctgctgggcaaccccgagtgcgaccctctgctgcccgtgagatcctggtcctacattgtggaaacccctaacagcgagaacggcatctgctaccctggcgacttcattgattacgaggagctgcgggagcagctgtcctcagtgagcagcttcgagaggtttgagatcttccccaaggagtcctcttggcccaaccacaacacaaacaagggcgtcaccgccgcctgcagccatgagggaaagagcagcttctacagaaacctgctgtggctgacagagaaggagggctcctaccccaagctgaagaattcctacgtgaacaagaaaggcaaagaggtgctggtgctgtgggggatccaccaccctcccaattccaaggaacagcagaacctgtaccagaacgaaaacgcctacgtctctgtggtgacctccaactacaacagaagattcacccctgagatcgcagagcggcccaaggtgcgggaccaagccggcagaatgaactactactggaccctgctgaagcctggcgacacaatcatcttcgaggccaatggaaacctgatcgccccaatgtacgccttcgccctgtcccggggcttcggaagcggcatcatcacaagcaacgcctccatgcacgagtgcaacaccaagtgccagaccccactgggcgccatcaattcctctctcccttaccagaacatccaccctgtgaccatcggggaatgccccaagtacgtgagatccgccaagctgcggatggtgaccggcctgagaaacatccccagcattcagagcaggggcctgtttggagccatcgctggcttcatcgagggcggatggaccggcatgatcgacggctggtacggctaccaccaccagaatgaacagggctccggctacgcagccgatcagaaaagcacacagaatgccatcaacggcatcaccaacaaagtgaacaccgtgatcgaaaagatgaacatccagttcaccgccgtgggcaaggagttcaacaagctggagaagagaatggagaacctgaacaagaaggtggacgacggctttctggacatttggacatacaatgctgagctgctggtgctgctggagaatgagaggaccctggactttcacgactccaacgtgaaaaacctgtacgaaaaggtcaagagccagctgaagaacaatgccaaggagatcggcaacggctgcttcgagttctaccacaagtgcgacaacgagtgtatggagtccgtgagaaacggcacctatgactaccccaagtactctgaggagtccaaactgaacagagagaaggtggacggagtgaagctggagtct

SEQ ID NO: 15 - protein, 507 aa, synthetic construct
DTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSRGLFGAIAGFIEGGWTGMIDGWYGYHHQNEQGSGYAADRKSTQNAIDGITNKVNSVIEKMNTQFTSVGKEFNHLEKRIENLNRKVDDGFLDVWTYNAELLVLLENERTLDYHDSNVKNLYEKVRSQLKNNAKEIGNGCFEFYHKCDDSCMESVKNGTYDYPKYSEESKLNREEIDGVKLES

SEQ ID NO: 16 - DNA, 1521 bp, synthetic construct
  source 1..1521: mol_type = other DNA
gacacaatctgcattggctaccatgccaacaacagcacagacaccgtggacaccgtcctggagaaaaacgtgacagtgacccacagcgtgaacctgctggaagactcccacaatggaaaactgtgcaagctgaagggcatcgcccctctgcagctgggcaagtgcaacatcgccggctggctgctgggcaacccagagtgcgacctgctgctgacagccagtagctggagctacatcgtggaaaccagcaacagcgaaaatggcacatgctaccctggcgacttcatcgattacgaggagctgcgggagcagctgagcagcgtcagctctttcgagaagtttgagatcttccccaaaacctcaagctggcccaaccacgaaaccacaaagggcgtgaccgccgcctgcagctacgcaggcgccagctctttctacagaaacctgctgtggctgacaaagaaggggagcagctaccccaagctctccaaaagctacgtgaacaacaagggcaaggaggtgctggtgctatggggggtgcatcaccctccaaccggcaccgaccagcagagcctgtaccagaacgccgacgcctacgtctccgtgggctcctccaagtacaacagaagattcacccctgagatcgctgctcggcccaaggtgcgggaccaggccggaaggatgaactactactggaccctgctggagcctggcgacacaatcaccttcgaggccacaggcaacctgatcgccccttggtacgccttcgctctcaacagaggctccggcagcggcatcatcacctccgacgcccctgtgcacgactgcaacacaaagtgccagacccctcacggggccatcaactccagcctgcctttccagaacatccaccctgtgaccatcggagaatgccccaagtacgtgagaagcaccaagctgagaatggccacaggcctgagaaacatcccttccatccagagcaggggcctgtttggggccatcgccggcttcatcgagggcggctggaccggcatgatcgacggctggtacggctaccaccatcagaacgaacagggctccggctacgccgccgacagaaagtccacccagaatgccattgacggcatcaccaacaaggtgaattccgtgatcgagaagatgaacacccagttcaccagcgtgggaaaagagttcaaccacctggagaagagaatcgagaacctgaacaggaaggtggatgacggcttcctggacgtgtggacatacaacgctgaactgctggtgctgctggaaaacgaaagaaccctggactaccacgacagcaacgtgaagaacctgtacgagaaggtgcggagccagctgaagaacaacgccaaggaaatcggcaatggctgtttcgaattctaccacaagtgcgacgacagctgtatggaatccgtgaaaaacggcacatacgactaccccaagtacagcgaggagagcaagctgaacagagaggaaatcgatggcgtgaaactggagtcc

SEQ ID NO: 17 - protein, 508 aa, synthetic construct
ADTLCIGYHANNSTDTVDTVLEKNVTVTHSVNLLENRHNGKLCKLRGVAPLHLGKCNIAGWLLGNPECESLSTASSWSYIVETSNSDNGTCYPGDFINYEELREQLSSVSSFERFEIFPKTSSWPNHDTNRGVTAACPHDGTNSFYRNLIWLVKKGNSYPKINKSYINNKEKEILVLWAIHHPSTSADQQSLYQNADAYVFVGSSRYSRKFEPEVATRPKVRDQAGRMNYYWTLVEPGDKITFEATGNLVVPRYAFALKRNSGSGIIISDTSVHDCDTNCQTPNGAINTSLPFQNIHPVTIGECPKYVKSTKLRMATGLRNIPSIQSRGLFGAIAGFIEGGWTGMIDGWYGYHHQNEQGSGYAADLKSTQNAIDGITNKVNSVIEKMNTQFTAVGKEFSHLERRIENLNKKVDDGFLDIWTYNAELLVLLENERTLDYHDSNVKNLYEKVRSQLKNNAKEIGNGCFEFYHKCDDMCMESVKNGTYDYPKYSEEAKLNREEIDGVKLES

SEQ ID NO: 18 - DNA, 1524 bp, synthetic construct
  source 1..1524: mol_type = other DNA
gctgacaccctgtgcattggctaccatgccaacaacagcaccgacaccgtcgacacagtcctggaaaagaacgtgacagtgacccactccgtgaatctgctggaaaacagacacaacggcaaactgtgcaagctgcggggcgtggcccctctgcacctgggcaaatgcaacatcgccggatggctgctgggcaacccagagtgcgaaagcctgagcaccgccagctcttggtcctacatcgtcgaaaccagcaacagcgacaatggcacatgctaccctggcgacttcatcaactacgaggaactgagagagcagctgagctctgtgtcctccttcgagaggtttgagatcttccccaagacctcaagctggcccaatcacgacacaaacagaggagtgaccgccgcctgtcctcacgatggaaccaacagcttctacagaaatctgatctggctcgtgaagaagggcaactcttaccccaagatcaacaagtcttacatcaacaacaaagagaaggaaattctggtgctgtgggccatccaccaccccagcacctccgctgaccagcaaagcctgtaccagaacgctgacgcctacgtcttcgtgggcagctcccggtacagcagaaagttcgaacctgaagtggccacccggcccaaggtgcgggaccaggccggcagaatgaactactactggaccctggtcgaacctggcgacaagatcaccttcgaagccaccggaaacctggtggtgcccagatacgccttcgccctgaagagaaattcgggcagcggcatcatcatcagcgacacatccgtccacgactgcgacaccaactgtcagaccccaaatggggccatcaacaccagcctgcctttccagaacatccaccctgtcaccatcggagagtgccccaagtacgtgaagtccacaaagctgagaatggccaccggcctgcggaacatccccagcatccagagccggggactgtttggcgccatcgccggcttcatcgagggcggctggaccggcatgatcgacggctggtacggctaccaccatcagaatgagcagggcagcggctacgccgctgacctgaagtccacacagaacgccatcgacggcatcaccaacaaggtgaacagcgtgatcgagaaaatgaacacccagttcaccgccgtgggcaaggaattctcccacctggagagacggattgagaacctgaacaagaaggtggacgacggctttctggacatctggacctacaacgctgaactgctggtgctgctggaaaatgagagaacactggactaccacgattccaatgtcaaaaacctgtatgagaaagtgcggagccagctgaagaacaatgccaaggaaattggcaacggctgtttcgagttctaccacaagtgcgatgatatgtgtatggagtccgtgaagaatggaacatacgactaccccaagtacagcgaagaggccaagctgaacagagaggagatcgacggggtgaagctggagtct

SEQ ID NO: 19 - protein, 500 aa, synthetic construct
TATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVKQNGTSSACIRKSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIQSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKS

SEQ ID NO: 20 - DNA, 1500 bp, synthetic construct
  source 1..1500: mol_type = other DNA
accgccaccctgtgcctggggcaccacgctgtgccaaatggcaccattgtgaagaccattaccaatgacagaatcgaggtgaccaatgccaccgaactggtgcagaattccagcattggcgagatctgcgacagccctcaccagatcctggatggcgagaactgcaccctgatcgatgctctgctgggcgaccctcagtgcgatggcttccagaacaagaaatgggacctgttcgtggagcggagcaaggcctacagcaactgctacccctacgacgtccccgattacgccagcctgcggagcctggtggcctcctccggcacactggagttcaacaacgaaagcttcaactggacaggagtgaagcagaacggcacatccagcgcctgcatcagaaaaagctccagcagcttcttctctcgactgaactggctgacccacctgaactacacctaccccgccctgaacgtgaccatgccaaacaatgagcagtttgacaaactgtacatttggggagtgcaccaccccggcacagacaaggaccagatctttctgtacgcccagagcagcggcaggatcaccgtgtctaccaagagaagccagcaggccgtgatccccaacatcggcagcagacctcggatcagagacatcccctccagaatctccatctactggacaattgtgaagcctggagacattctgctgatcaacagcaccggcaacctgatcgcccctcgaggatacttcaagatccagagcggcaaaagcagcatcatgagatctgacgcccccatcggcaaatgcaaaagcgagtgtatcacccccaacggcagcatccccaacgacaagccctttcagaacgtgaacagaatcacctatggcgcctgtcccaggtacgtgaagcacagcaccctgaagctggccacaggaatgagaaacgtccccgagaagcaaacccggggcatctttggcgctattgccggcttcatcgagaacggctgggaagggatggtggacggctggtacggcttccggcaccagaacagcgagggaagaggccaggccgctgacctgaagtccacccaggccgccattgatcagatcaacggcaagctgaacagactgatcggcaagaccaacgaaaaattccaccagatcgaaaaggaattcagcgaagtggagggcagaattcaggacctggaaaagtacgtggaggacacaaagatcgacctgtggtcctacaacgctgaactgctggtggccctggagaatcagcacaccatcgacctgaccgacagcgagatgaacaaactgtttgaaaagaccaagaagcagctgagagaaaacgccgaggacatgggcaatggatgcttcaaaatctaccacaagtgcgacaacgcctgtattggcagcatcagaaatggcacctacgaccacaacgtctacagggacgaggccctgaacaacagatttcaaatcaagggcgtggagctgaagtct

SEQ ID NO: 21 - protein, 500 aa, synthetic construct; identical to SEQ ID NO: 19

SEQ ID NO: 22 - DNA, 1500 bp, synthetic construct
  source 1..1500: mol_type = other DNA
acagccacactctgtctgggccatcacgctgtgccaaacggcacaatcgtgaagacaatcaccaacgaccagattgaggtgacaaatgccacagagctggtgcagagctccagcacaggcggcatctgcgacagccctcatcagatcctggatggcgagaactgcacactgatcgatgccctgctgggcgaccctcagtgcgacggcttccagaacaagaaatgggacctgttcgtggagcggagcaaggcttacagcaactgttacccctacgacgtccctgactacgcctccctgcggagcctggtggcctcctccggcaccctggagtttaacaatgaatcctttaactggacaggcgtcacccagaacggcacatcttccagctgcaagagaagaagcaacaactccttcttcagcagactgaattggctgacccacctgaagttcaagtaccctgccctgaacgtgaccatgccaaacaatgagaagttcgacaagctgtacatctggggggttcaccaccctggcacaaacaacgaccagatcagcctgtacacccaggccagcggccggatcaccgtgtctacaaagcggagccagcagacagtgatccccaacatcggctccagacctcgggtgagagacatcccctccagaatcagcatctactggaccatcgtgaagccaggcgacatcctgctcatcaacagcaccggaaacctgattgcacccagaggctacttcaagatcagaagcggcaagagcagcatcatgagaagcgatgcccccatcggcaagtgcaacagcgagtgtatcacccccaacggcagcatccccaatgacaagcccttccagaacgtgaacagaatcacctatggcgcctgccccaggtacgtcaagcagaacaccctgaagctggccaccggaatgagaaacgtccccgagaagcagaccagggggatctttggagccatcgccggcttcatcgagaacggctgggagggaatggtggacggatggtacggcttccggcaccagaactctgagggcattggccaggccgcagatctgaagtccacccaggccgccatcaaccagatcaacggcaagctgaacagactgatcggcaagacaaatgaaaaattccaccagatcgagaaagaattcagcgaggtggaaggccggattcaggacctggagaagtacgtggaggacaccaagatcgacctgtggagctacaacgctgaactgctcgtggccctggagaaccagcacacaatcgacctgacagacagcgagatgaacaagctgttcgaaagaacaaagaaacagctgagagaaaacgccgaggacatgggcaacggctgcttcaagatctaccacaagtgcgacaacgcctgcattggcagcatcagaaacggcacctacgaccatgacgtttaccgggacgaggccctgaacaaccggttccagatcaagggcgtggaactgaagtcc

SEQ ID NO: 23 - protein, 500 aa, synthetic construct
TATLCLGHHAVPNGTIVKTITNDRIEVTNATELVQNSSIGEICNSPHQILDGGNCTLIDALLGDPQCDGFQNKEWDLFVERSRANSSCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQFSLFAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKS

SEQ ID NO: 24 - DNA, 1500 bp, synthetic construct
  source 1..1500: mol_type = other DNA
acagccacactgtgcctgggccatcacgctgtgcccaacggaaccatcgtgaagaccattaccaacgacagaatcgaagtgacaaatgccacagagctggtgcagaacagcagcattggcgagatctgcaacagccctcaccagatcctggacggaggcaactgcacactgatcgatgccctgctcggcgaccctcagtgcgatggcttccagaacaaggagtgggacctgttcgtggaaagaagcagagccaacagctcctgctacccctacgacgtccctgactacgccagcctgcggagcctggtggcctccagcggcaccctggagttcaaaaacgaaagcttcaattggaccggagtgaaacagaacggcacctccagcgcctgtatcagaggctccagcagctctttcttcagcagactgaactggctgacaagcctgaacaacatttaccccgcccagaacgtgacaatgcccaacaaggagcagtttgacaaactgtacatctggggagtgcaccaccctgataccgacaaaaaccagttctccctgttcgcccagtcttccggcagaatcaccgtgtctaccaagcggagccagcaggccgtgatccccaacatcggcagccggccccggattcgggacatcccttcccggatcagcatttactggaccatcgtgaagcctggagacattctcctgatcaacagcaccggcaacctgatcgcccctcggggctacttcaagatcagaagcggcaagtccagcatcatgagatctgacgcccctatcggcaagtgcaagtctgagtgtatcacacccaacggcagcatccccaacgacaagccctttcagaacgtgaacagaatcacctatggcgcctgtcccaggtacgtgaagcagagcaccctgaagctggccacagggatgagaaacgtccccgaaaagcagacccggggcatctttggcgctatcgccggcttcatcgagaacggctgggagggcatggtggacggctggtacggcttccggcaccagaacagcgagggcagaggccaggccgctgacctgaagtccacccaggctgccatcgaccagatcagcggaaagctgaacagactgatcggcaaaacaaacgaaaagttccatcagatcgagaaggagttctccgaggtggaaggccgggtgcaggacctggaaaagtacgtggaagacaccaagatcgacctgtggtcctacaacgctgaactgctggtcgccctggagaatcagcacaccatcgacctgaccgacagcgagatgaacaaactgttcgagaagaccaagaagcagctgcgggaaaacgccgaggatatgggcaacggatgcttcaagatttaccacaagtgcgacaacgcctgcattggcagcatcagaaatgagacatacgaccacaatgtttaccgggatgaggccctgaacaacagattccagatcaagggagtggagctgaagtcc

SEQ ID NO: 25 - protein, 500 aa, synthetic construct
TATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNYTYPALNVTMPNNEQFDKLYIWGVHHPSTDKDQISLFAQPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKS

SEQ ID NO: 26 - DNA, 1500 bp, synthetic construct
  source 1..1500: mol_type = other DNA
accgccacactgtgcctgggccatcacgctgtgccaaacggcaccatcgtgaagaccatcaccaacgatagaatcgaggtgaccaatgccacagaactggtgcagaatagcagcattggcgagatctgcgactccccccaccagatcctggacggaggaaactgcacactgatcgatgccctgctcggcgaccctcagtgcgatggctttcagaacaaggagtgggacctgttcgtggagcggagcagagccaacagcaactgttacccctacgacgtccctgactacgccagcctgcggagcctggtggcctccagcggcacactggagttcaagaatgagagcttcaactggaccggagtgaaacagaacggcacaagcagcgcctgcatcagaggctccagctcatccttcttcagcagactgaactggctgacccacctgaactacacctaccctgccctgaacgtgacaatgccaaacaatgaacagtttgacaagctgtacatctggggagtgcaccacccttccaccgacaaagatcagatttctctgttcgctcagccaagcggcagaatcaccgtgtctaccaagcggagccagcaggccgtgattcctaacatcggcagcagacctcggatcagagacatccccagcagaatctcaatctactggacaatcgtgaagcctggcgacatcctgctgatcaattccacaggaaacctgattgcacctcggggctacttcaagatcaggagcggcaagagcagcatcatgagaagcgatgctcccatcggcaagtgcaagagcgagtgtatcacccccaacggcagcatccccaatgacaagccttttcagaacgtgaacagaatcacctatggcgcctgccccaggtacgtcaagcagtcaacactgaagctggccacagggatgagaaacgtccctgagaagcaaacacggggaatctttggcgctatcgccggcttcatcgagaatggctgggaaggcatggtggacggctggtacggcttccggcatcagaacagcgagggaagaggccaggccgctgacctgaagtcaacacaggccgccatcgaccagatcaatggcaagctgaacaggctgatcggcaagacaaacgaaaagtttcaccagatcgagaaggagttcagcgaagtggagggcagagtgcaggacctggagaagtacgtggaggataccaagatcgacctgtggtcctacaacgcagagctgctcgtggccctggagaaccagcacaccattgacctgaccgacagcgagatgaacaagctgttcgagaagaccaagaaacagctgagagagaatgccgaggacatgggcaacggctgcttcaagatctaccacaagtgcgacaacgcctgcattggaagcatcagaaacgaaacctacgatcacaacgtctacagagacgaggctctgaacaaccggttccagatcaagggcgtggagctgaagtcc

SEQ ID NO: 27 - protein, 500 aa, synthetic construct
TATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFTNEGFNWTGVAQDGTSYACKRGSVKSFFSRLNWLHKLEYKYPALNVTMPNNDKFDKLYIWGVHHPSTDSDQTSLYVQASGRVTVSTKRSQQTVIPNIGSRPWVRGISSRISIYWTIVKPGDILLIKSTGNLIAPRGYFKIRSGKSSIMRSDAPIGNCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKS

SEQ ID NO: 28 - DNA, 1500 bp, synthetic construct
  source 1..1500: mol_type = other DNA
acagccaccctgtgcctgggacaccacgctgtgccaaatggcaccctggtgaagacaatcaccaacgaccagatcgaagtgacaaatgccacagagctggtgcagagctcctccaccggcagaatctgcgactctcctcacagaatcctggacggcaagaactgcaccctgatcgatgccctgctgggcgaccctcactgcgacggcttccagaacaaggagtgggacctgttcgtggagagaagcaaggcctactccaactgttacccttacgacgtccctgattacgcctccctgagaagcctggtggcctcctctggcaccctggagttcaccaatgagggcttcaactggaccggagtggcccaggatggcacctcctacgcctgcaagagaggctccgtgaaaagcttcttcagcagactgaactggctccacaagctggagtacaagtaccccgccctgaacgtgaccatgccaaacaacgacaagttcgacaagctctacatctggggcgtccaccaccccagcaccgacagcgaccagacatctctctatgtccaggccagcggcagagtgacagtcagcaccaagagaagccagcaaacagtgatccccaacatcggcagcagaccttgggtgcggggcatctcttccagaatcagcatctactggacaatcgtgaagcctggcgatatcctgctgatcaagagcaccggcaacctgatcgcccccagaggctacttcaagatcagaagcggaaagagctccatcatgagaagcgatgctcctatcggcaactgcaactctgagtgtatcacccccaacggctccattcccaacgacaagccttttcagaacgtgaacagaatcacctacggggcctgccccaggtacgtgaagcagaacaccctgaagctggccaccggaatgagaaacgtccccgagaagcagacacggggcatctttggcgccatcgccggcttcatcgagaacggctgggaggggatggtggatggctggtacggcttccggcaccagaattcggagggcaccggccaggccgccgacttgaaatccacccaggccgccatcaaccagatcaacggcaagctgaacagactgatcgagaagacaaatgagaagttccaccagatcgagaaggagttcagcgaagtggagggaagaatccaggacctggagaagtacgtggaagacaccaagatcgacctgtggtcctacaacgcagaactgctggtggccctggaaaaccaacacacaatcgacctgacagacagcgaaatgaacaaactgttcgagagaaccagaaaacagctgagagagaacgccgaggacatgggcaacggctgtttcaagatctaccacaagtgcgacaacgcctgcattggctccatcagaaacggcacctacgatcacgatgtttaccgggacgaggccctgaacaatcgtttccagatcaagggagtggagctgaagtct

SEQ ID NO: 29 - protein, 500 aa, synthetic construct
TATLCLGHHAVPNGTLVKTITNDQIEVTNATELVQSSSTGRICDSPHRILDGKNCTLVDALLGDPHCDGFQNEKWDLFVERSKAFSNCYPYDVPDYASLRSLVASSGTLEFINESFNWTGVTQSGGSYACKRGSDNSFFSRLNWLYESESKYPVLNVTMPNNGNFDKLYIWGVHHPSTDKEQTNLYVRASGRVTVSTKRSQQTIIPNIGSRPWVRGLSSRISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTRGIFGAIAGFIENGWEGMVDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKS

SEQ ID NO: 30 - DNA, 1500 bp, synthetic construct
  source 1..1500: mol_type = other DNA
accgccaccctgtgcctgggccaccacgctgtgccaaacggcaccctggtgaagaccatcaccaacgatcagattgaagtgaccaatgccacagagctggtgcagagctcctccactggcagaatctgcgactcccctcacagaatcctggatggcaaaaactgcacactcgtggatgccctgctgggcgacccccactgcgacggcttccagaatgagaagtgggacctgttcgtggagagaagcaaggccttcagcaactgctacccttacgacgtccctgactacgccagcctgagaagcctggtggcctccagcggcacactggagttcatcaatgagtccttcaactggacaggcgtgacacagtctggcggctcttacgcctgcaagcggggcagcgacaacagcttcttctcccggctgaattggctgtacgagagcgagagcaagtaccctgtgctgaacgtgaccatgccaaacaatggcaatttcgacaaactgtacatctggggagtgcaccaccccagcaccgacaaggagcagaccaacctctacgtgagagccagcggcagagtgaccgtgtccacaaagcggagccagcagaccatcatccccaacatcggcagccggccctgggtgagaggcctgagctcccggatcagcatctactggacaatcgtgaaacccggagacatcctgctgatcaacagcaacggcaacctgatcgcccccagaggctactttaagatcagaacaggcaagagctccatcatgagaagcgatgcccccatcggcacatgcagcagcgagtgtatcacccccaacggctcaatccccaacgacaagcctttccagaacgtgaacaagattacctatggcgcctgccccaagtacgtgaagcagaacacactgaagctggccacagggatgaggaatgtccccgaaaagcagacaagaggcatctttggggccatcgccggcttcatcgagaacggctgggaaggaatggtggacggctggtacggcttcaggcaccagaacagcgagggcaccggccaggccgctgacctgaagtccacccaggccgccatcgaccagatcaatggaaagctgaacagagtgatcgagaagacaaacgaaaagttccaccagatcgaaaaggaattctccgaggtggaaggacggattcaggacctggaaaagtacgtggaagacaccaagatcgacctgtggagctacaacgcagagctgctggtggctctggagaaccagcacacaatcgacctgacagacagcgagatgaacaaactgttcgaaaagacacggagacagctgagggagaacgctgaagacatgggaaacggctgctttaagatttaccacaagtgcgacaatgcctgcattggcagcattcggaacggcacatacgaccatgatgtctacagagacgaggctctgaacaaccggttccagatcaagggcgtggagctgaagtct

SEQ ID NO: 31 - protein, 500 aa, synthetic construct
TATLCLGHHAVPNGTLVKTITNDQIEVTNATELVQSSSTGKICDNPHRILDGINCTLIDALLGDPHCDGFQNEKWDLFVERSKAFSNCYPYDVPDYASLRSLVASSGTLEFINEGFNWTGVTQNGGSSACKRGPDNGFFSRLNWLYKSGSTYPVQNVTMPNNDNSDKLYIWGVHHPSTDKEQTDLYVQASGKVTVSTKRSQQTVIPNVGSRPWVRGLSSRVSIYWTIVKPGDILVINSNGNLIAPRGYFKMRTGKSSIMRSDAPIGTCSSECITPNGSIPNDKPFQNVNKITYGACPKYVKQNTLKLATGMRNVPEKQTRGIFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKS

SEQ ID NO: 32 - DNA, 1500 bp, synthetic construct
  source 1..1500: mol_type = other DNA
accgccaccctgtgcctcgggcaccacgctgtgccaaacggcaccctggtgaagaccatcaccaacgatcagatcgaagtgaccaatgccaccgaactggtgcagagctcctccaccggcaagatctgcgacaaccctcacagaatcctggacggcatcaactgcaccctgatcgatgccctgctgggcgaccctcactgcgatggcttccagaatgagaagtgggacctgttcgtcgagagaagcaaggccttttccaactgttacccttacgacgtccccgattacgccagcctgagatccctggtggcctccagcggaacactggagttcatcaacgagggcttcaactggacaggcgtgacccagaacggcggctcctctgcctgcaagagaggccccgacaatggctttttcagcagactgaactggctgtacaagagcggcagcacctacccagtccagaacgtgaccatgccaaacaacgacaactccgacaagctgtacatctggggcgtccaccaccccagcacagacaaggagcagacagacctctacgtccaagcctccggcaaggtcaccgtgtccaccaagcggagccagcagaccgtgatccccaacgtgggcagccggccctgggtgagaggcctgagctcccgggtgtccatctactggaccatcgtgaaaccaggcgacatcctggtgatcaacagcaacggcaacctgatcgcccctcggggctacttcaagatgagaaccggcaagagcagcatcatgagaagcgatgccccaatcggaacatgctcctctgaatgcattacacccaacggaagcatccccaacgacaaacccttccagaacgtgaacaagattacatatggcgcctgccccaagtacgtgaagcagaacacactgaagctggccacaggaatgagaaacgtccctgagaagcagacaagaggcatctttggcgctatcgccggattcatcgaaaacggctgggagggaatgatcgacggctggtacggatttagacaccaaaacagcgagggcaccggccaggccgctgacctgaagtcaacccaggccgccatcgaccagatcaacggcaagctgaacagagtgatcgaaaagaccaatgagaagttccatcagatcgagaaggagttcagcgaagtggagggcagaatccaggacctggagaagtacgtggaggataccaagatcgacctgtggagctacaacgcagagctgctggtggccctggagaaccagcacaccatcgacctgacagacagcgagatgaacaagctgttcgagaagacaagaagacagctgagagagaacgccgaggatatgggcaacggctgtttcaagatctatcacaagtgcgacaacgcctgcattggcagcatcagaaacggcacctacgaccacgatgtctacagagacgaggctctgaacaacagattccagatcaagggcgtggagctgaagtcc

SEQ ID NO: 33 - protein, 500 aa, synthetic construct
TATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTRGLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKS

SEQ ID NO: 34 - DNA, 1500 bp, synthetic construct
  source 1..1500: mol_type = other DNA
acagccaccctgtgcctgggccaccacgctgtgccaaacggcacactggtgaagacaatcaccgacgaccagatcgaggtgaccaatgcaacagagctggtgcagagttccagcaccggaaagatctgcaacaaccctcaccggatcctggacggcatcgactgcaccctgattgatgccctgctcggcgaccctcactgcgacgtgttccagaacgaaacatgggacctgttcgtggagcggtccaaggcctttagcaactgctacccctacgacgtccccgattacgccagcctgagatctctcgtggcctccagcggcaccctggagttcattacagagggcttcacatggacaggcgtgacacagaacggcggcagcaacgcctgcaagagaggccctggctctggcttcttcagcaggctgaattggctgacaaagagcggaagcacctaccctgtgctgaacgtgaccatgccaaacaacgacaatttcgacaagctgtacatctggggcgtccaccaccccagcaccaaccaggagcagaccagcctctatgtccaggccagcggcagagtgacagtgagcaccagacggtcccagcagaccatcatccctaacatcggatccagaccttgggtgcggggcctgagcagcagaatctccatctactggaccatcgtgaaacctggcgatgtcctggtgatcaactccaacggcaacctgatcgcccctcggggctacttcaagatgcggacaggcaagagctcaatcatgagaagcgatgctcctatcgacacctgcatcagcgagtgtatcacacccaacggctctatccccaacgacaagcccttccagaacgtgaacaagatcacatatggagcctgccccaagtacgtgaaacagaacaccctgaagctggccaccgggatgagaaacgtccctgagaagcagacccggggcctgtttggagccatcgccggcttcatcgagaacggctgggaaggcatgatcgacggctggtacggattccggcaccagaacagcgaaggcaccggccaggctgctgacctgaagtccacacaggccgccatcgaccagatcaatggcaagctgaacagagtgatcgaaaagacaaacgaaaagttccaccagatcgaaaaggagttcagcgaagtggagggcagaatccaggacctggagaagtacgtggaggacaccaagatcgacctgtggagctacaacgcagaactgctggtcgccctggagaaccagcacaccatcgacctgacagattcggagatgaacaagctgttcgagaaaacccggagacagctgagagagaacgccgaggacatgggaaacggctgcttcaagatttaccacaagtgcgacaacgcctgcattgaatccattcggaacggcacctacgaccacgatgtctacagagacgaggccctgaacaaccggtttcagatcaagggcgtggagctgaagtcc

SEQ ID NO: 35 - protein, 499 aa, synthetic construct
ATLCLGHHAVPNGTLVKTITDDQIEVTNATELVQSSSTGGICNSPHQILDGKNCTLIDALLGDPHCDDFQNKEWDLFVERSTAYSNCYPYYVPDYATLRSLVASSGNLEFTQESFNWTGVAQGGSSYACRRGSVNSFFSRLNWLYNLNYKYPEQNVTMPNNDKFDKLYIWGVHHPGTDKDQTNLYVQASGRVIVSTKRSQQTVIPNIGSRPWVRGVSSIISIYWTIVKPGDILLINSTGNLIAPRGYFKIQSGKSSIMRSDAHIDECNSECITPNGSIPNDKPFQNVNKITYGACPRYVKQNTLKLATGMRNVPEKQTRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAEILVALENQHTIDLTDSEMSKLFERTRRQLRENAEDMGNGCFKIYHKCDNACIGSIRNGTYDHDIYRNEALNNRFQIKGVQLKS

SEQ ID NO: 36 - DNA, 1497 bp, synthetic construct
  source 1..1497: mol_type = other DNA
gccaccctgtgtctcgggcaccacgcagtccccaacggcaccctggtgaagacaatcacagatgaccagatcgaagtgaccaatgccacagagctggtgcagagcagctccaccggaggcatctgcaattcaccccaccagatcctggacggcaagaactgcaccctgatcgatgccctgctgggcgaccctcactgcgacgactttcagaacaaggagtgggacctgttcgtggagagaagcaccgcctacagcaactgctacccttactacgtccctgactacgccacactgaggagcctggtggcctccagcggcaacctggaattcacccaggagagcttcaattggacaggagtggcccagggcggcagctcctatgcctgccggagaggctccgtgaacagcttcttcagcagactgaactggctgtacaatctgaactacaagtaccccgaacagaacgtgaccatgccaaacaacgacaagttcgacaagctgtacatctggggagtgcaccaccccggcaccgacaaggatcagacaaacctctacgtccaggcttctggccgggtgatcgtgtccaccaagagaagccagcagacagtgatccccaacatcggcagccggccttgggtccggggcgtgtccagcatcatcagcatctactggacaatcgtgaagccaggcgacatcctgctgatcaattccaccggcaacctgatcgcccccagaggctacttcaagatccagagcggcaaaagcagcatcatgagaagcgatgcccacattgatgaatgcaactctgagtgtatcacccccaacggcagcatccccaatgacaagcccttccagaacgtgaacaagatcacctatggcgcctgccccaggtacgtgaagcagaacacactgaagctggccacaggaatgagaaatgtccccgagaagcagacccggggcatctttggagccatcgccggcttcatcgaaaacggctgggaaggaatggtggacggctggtacggcttccggcaccagaacagcgagggcacaggccaggccgctgacctgaagtcaacacaggccgccatcaatcagatcacaggcaagctgaacagagtgatcaagaagaccaatgagaagttccaccagatcgagaaggagttctccgaagtggagggccggatccaggacctggaaaagtacgtggaggacacaaagattgacctgtggtcttacaacgccgagatcctggtggccctggaaaaccagcacaccatcgacctgaccgacagcgagatgtccaagctgtttgagagaacacggcggcagctgagagaaaacgccgaggacatgggcaacggctgcttcaagatttaccacaagtgcgacaacgcctgcattggcagcatcagaaacggcacatacgaccacgacatttacagaaatgaggccctgaacaacagatttcagatcaagggagtgcagctgaagtct

SEQ ID NO: 37 - protein, 694 aa, synthetic construct
TDNLWVTVYYGVPVWKDADTTLFCASDAKAHETEVHNIWATHACVPTDPNPQEIPLENVTENFNMWKNNMVEQMQEDVISLWDQSLKPCVKLTPLCVTLNCTNANLTNVNISISVSNVSIGNITDEVSNCSFDVTTEITDRKQRVHALFYKLDLVPMNKDESSEYILIHCNTSVIKQACPKISFDPIPIHYCTPAGYAILKCNDKNFNGTGPCKNVSSVQCTHGIKPVVSTQLLLNGSLAEEEIIIRSKNLTDNAKTIIVHLNKSVVIDCTRPSNNTRTGMTIGPGRVFYKTGEIVGDIRKAYCQINRTNWFNALKQVAEKLKGHFNNKTIIFQPPSGGDPEITMHHFNCRGEFFYCNTSQLFNFTYNGTQTTEGKDNITLPCRIKQVIKMWQRVGQAMYAPPISGIINCVSNITGILLTRDGGDNDSTNETFRPGGGDMRDNWRSELYKYKVVQIEPLGIAPTKCKRRVVEGGSGGGGSGGGGSGGGGSGGAVGIGALIFGFLGAAGSTMGAASITLTVQARQLLSGIVQQQSNLLRAIEAQQHMLQLTVWGIKQLQARVLAVERYLKDQQFLGLWGCSGKIICCTAVPWNSTWSNKSLEEIWNNMTWIEWEREISNYTSKIYEILTESQNQQDRNEKDLLELDGSLEVLFQGPGSGYIPEAPRDGQAYVRKDGEWVLLSTFLHHHHHHHHHH

SEQ ID NO: 38 - protein, 472 aa, synthetic construct
TDNLWVTVYYGVPVWKDADTTLFCASDAKAHETEVHNIWATHACVPTDPNPQEIPLENVTENFNMWKNNMVEQMQEDVISLWDQSLKPCVKLTPLCVTLNCTNANLTNVNISISVSNVSIGNITDEVSNCSFDVTTEITDRKQRVHALFYKLDLVPMNKDESSEYILIHCNTSVIKQACPKISFDPIPIHYCTPAGYAILKCNDKNFNGTGPCKNVSSVQCTHGIKPVVSTQLLLNGSLAEEEIIIRSKNLTDNAKTIIVHLNKSVVIDCTRPSNNTRTGMTIGPGRVFYKTGEIVGDIRKAYCQINRTNWFNALKQVAEKLKGHFNNKTIIFQPPSGGDPEITMHHFNCRGEFFYCNTSQLFNFTYNGTQTTEGKDNITLPCRIKQVIKMWQRVGQAMYAPPISGIINCVSNITGILLTRDGGDNDSTNETFRPGGGDMRDNWRSELYKYKVVQIEPLGIAPTKCKRRVVE

SEQ ID NO: 39 - protein, 20 aa, synthetic construct
GGSGGGGSGGGGSGGGGSGG

SEQ ID NO: 40 - protein, 153 aa, synthetic construct
AVGIGALIFGFLGAAGSTMGAASITLTVQARQLLSGIVQQQSNLLRAIEAQQHMLQLTVWGIKQLQARVLAVERYLKDQQFLGLWGCSGKIICCTAVPWNSTWSNKSLEEIWNNMTWIEWEREISNYTSKIYEILTESQNQQDRNEKDLLELD

SEQ ID NO: 41 - protein, 705 aa, synthetic construct
AQNLWVTVYYGVPVWRDADTTLFCASDAKAYKTEVHNVWATHACVPTDPNPQEIYLTNVTENFNMWKNKMVEQMHEDIISLWDQSLKPCVQLTPLCVTLNCSDVTNNTLRNATVNANANATVTGDMEGEMKNCSYNMTTAVRDKQKKVYSLFYRLDVVQISNNSSSSSEYRLINCNTSAITQACPKVTFEPIPIHYCAPAGFAILKCNEEGFNGTGPCKNVSTVQCTHGIKPVVSTQLLLNGSLAEKGVIIRSENISNNAKTIIVQLAEPVTINCTRPNNNTRKGIHIGPGRAFYATGDIIGDIRKAYCNVSRTQWNKTLAQVAAQLTKYWNKTINFTSSSGGDVEITTHSFNCGGEFFYCNTTNLFNSTWRGNKTVSNSTELDENGTITLPCRIKQIINMWQRTGQAMYAPPIQGVIKCVSNITGLLLTRDGGSDNNISSETFRPGGGDMRDNWRSELYKYKVVKLEPLGVAPNKCRRRVVEGGSGGGGSGGGGSGGGGSGGAVGLGAVFIGFLGAAGSTMGAASITLTAQARQLLSGIVQQQSNLLKAIEAQQHLLKLTVWGIKQLQARVLALERYLKDQQLLGIWGCSGKLICCTSVPWNSSWSNKSFEQIWNNMTWLEWDKEVSNYTQIIYELLEVSQNQQEKNEQDLLSLDGSLEVLFQGPGSGYIPEAPRDGQAYVRKDGEWVLLSTFLHHHHHHHHHH

SEQ ID NO: 42 - protein, 483 aa, synthetic construct
AQNLWVTVYYGVPVWRDADTTLFCASDAKAYKTEVHNVWATHACVPTDPNPQEIYLTNVTENFNMWKNKMVEQMHEDIISLWDQSLKPCVQLTPLCVTLNCSDVTNNTLRNATVNANANATVTGDMEGEMKNCSYNMTTAVRDKQKKVYSLFYRLDVVQISNNSSSSSEYRLINCNTSAITQACPKVTFEPIPIHYCAPAGFAILKCNEEGFNGTGPCKNVSTVQCTHGIKPVVSTQLLLNGSLAEKGVIIRSENISNNAKTIIVQLAEPVTINCTRPNNNTRKGIHIGPGRAFYATGDIIGDIRKAYCNVSRTQWNKTLAQVAAQLTKYWNKTINFTSSSGGDVEITTHSFNCGGEFFYCNTTNLFNSTWRGNKTVSNSTELDENGTITLPCRIKQIINMWQRTGQAMYAPPIQGVIKCVSNITGLLLTRDGGSDNNISSETFRPGGGDMRDNWRSELYKYKVVKLEPLGVAPNKCRRRVVE

SEQ ID NO: 43 - protein, 20 aa, synthetic construct
GGSGGGGSGGGGSGGGGSGG

SEQ ID NO: 44 - protein, 153 aa, synthetic construct
AVGLGAVFIGFLGAAGSTMGAASITLTAQARQLLSGIVQQQSNLLKAIEAQQHLLKLTVWGIKQLQARVLALERYLKDQQLLGIWGCSGKLICCTSVPWNSSWSNKSFEQIWNNMTWLEWDKEVSNYTQIIYELLEVSQNQQEKNEQDLLSLD

SEQ ID NO: 45 - protein, 695 aa, synthetic construct
ADNLWVTVYYGVPVWKEATTTLFCASDAKAYKEEAHNIWATHACVPTDPNPQEVKLENVTENFNMWKNNMVDQMHEDIISLWDQSLKPCVKLTPLCVTLNCIEWKNNSTNVTDDGNNSTNAPDDIGVKNCSFNITTEVRDKKKQVYALFYKLDVVQIDDSSNTSYRLINCNTSAITQACPKVSFEPIPIHYCAPAGFAILKCNDKTFNGTGPCTNVSTVQCTHGIKPVVSTQLLLNGSLAEGEIMIRSENLTNNAKTIIVQFNQSVEISCTRPNNNTRKSVRIGPGQAFYATGEIIGDIRQAYCNINKAKWNKTLQQVAKKLRDLLNKTTINFKPHSGGDLEITTHSFNCGGEFFYCNTSKLFNNSIGRNATTSNSTESITLPCKIKQIINMWQGVGKAMYAPPIEGLIKCESNITGLLLTRDGGNTNNQNETFRPGGGDMRDNWRSELYKYKVVRIEPLGLAPTRCKRRVVEGGSGGGGSGGGGSGGGGSGGAIGLGALFLGFLGAAGSTMGAASLTLTVQARQVLSGIVQQQNNLLRAIEAQQHLLQLTVWGIKQLQARILAVERYLRDQQLLGIWGCSGKHICCTTVPWNSSWSNKSLEYIWQNMTWMEWEKEIDNYTGLIYSLIEESQTQQEKNEKELLELDGSLEVLFQGPGSGYIPEAPRDGQAYVRKDGEWVLLSTFLHHHHHHHHHH

SEQ ID NO: 46 - protein, 473 aa, synthetic construct
ADNLWVTVYYGVPVWKEATTTLFCASDAKAYKEEAHNIWATHACVPTDPNPQEVKLENVTENFNMWKNNMVDQMHEDIISLWDQSLKPCVKLTPLCVTLNCIEWKNNSTNVTDDGNNSTNAPDDIGVKNCSFNITTEVRDKKKQVYALFYKLDVVQIDDSSNTSYRLINCNTSAITQACPKVSFEPIPIHYCAPAGFAILKCNDKTFNGTGPCTNVSTVQCTHGIKPVVSTQLLLNGSLAEGEIMIRSENLTNNAKTIIVQFNQSVEISCTRPNNNTRKSVRIGPGQAFYATGEIIGDIRQAYCNINKAKWNKTLQQVAKKLRDLLNKTTINFKPHSGGDLEITTHSFNCGGEFFYCNTSKLFNNSIGRNATTSNSTESITLPCKIKQIINMWQGVGKAMYAPPIEGLIKCESNITGLLLTRDGGNTNNQNETFRPGGGDMRDNWRSELYKYKVVRIEPLGLAPTRCKRRVVE

SEQ ID NO: 47 - protein, 20 aa, synthetic construct
GGSGGGGSGGGGSGGGGSGG

SEQ ID NO: 48 - protein, 153 aa, synthetic construct
AIGLGALFLGFLGAAGSTMGAASLTLTVQARQVLSGIVQQQNNLLRAIEAQQHLLQLTVWGIKQLQARILAVERYLRDQQLLGIWGCSGKHICCTTVPWNSSWSNKSLEYIWQNMTWMEWEKEIDNYTGLIYSLIEESQTQQEKNEKELLELD

SEQ ID NO: 49 - protein, 703 aa, synthetic construct
KGNLWVTVYYGVPVWKEANPILFCASDAKAYETEVHNVWATHACVPTDPDPQEMVLGNVTENFNMWKNNMVEQMHEDIISLWDQSLKPCVKLTPLCVTLSCTAANTTDNSNSTQSTNSSWERIENGEIQNCSFNITTNIRDKIQKQYALFNRLDLVPIDRTDNTSYRIISCNTSVITQACPKVTFEPIPIHYCAPAGFAILKCNEKKFNGTGPCTNVSTVQCTHGIRPVVSTQLLLNGSLAEGEIVIRSANLSNNAKTIIVHLNESVDMTCIRPNNNTRKSIHIGPGRAFYATDIIGDIRKAHCNISAAKWNTTLYRIAKKLREHFPNKTINFNKSSGGDPEIIMHSFNCGGEFFYCNTTQLFNSSWTDGQELNNTGQELNSTITLPCRIKQIINRWQEVGKAMYAPPISGPIKCTSNITGLLLTRDGGNSNSSGKNDTETFRPGGGDMRDNWRSELYKYKVVKIEPLGVAPTKCRRRVVQGGSGGGGSGGGGSGGGGSGGAVGIGAVFLGFLGAAGSTMGAASLTLTVQARQLLSGIVQQQNNLLRAIEAQQHLLQLTVWGIKQLQARVLAVERYLRDQQLLGIWGCSGKLICCTNVPWNASWSNKSLDAIWDNMTWMQWEKEIDNYTDFIYRLLEESQNQQEKNEQDLLELDGSLEVLFQGPGSGYIPEAPRDGQAYVRKDGEWVLLSTFLHHHHHHHHHH

SEQ ID NO: 50 - protein, 481 aa, synthetic construct
KGNLWVTVYYGVPVWKEANPILFCASDAKAYETEVHNVWATHACVPTDPDPQEMVLGNVTENFNMWKNNMVEQMHEDIISLWDQSLKPCVKLTPLCVTLSCTAANTTDNSNSTQSTNSSWERIENGEIQNCSFNITTNIRDKIQKQYALFNRLDLVPIDRTDNTSYRIISCNTSVITQACPKVTFEPIPIHYCAPAGFAILKCNEKKFNGTGPCTNVSTVQCTHGIRPVVSTQLLLNGSLAEGEIVIRSANLSNNAKTIIVHLNESVDMTCIRPNNNTRKSIHIGPGRAFYATDIIGDIRKAHCNISAAKWNTTLYRIAKKLREHFPNKTINFNKSSGGDPEIIMHSFNCGGEFFYCNTTQLFNSSWTDGQELNNTGQELNSTITLPCRIKQIINRWQEVGKAMYAPPISGPIKCTSNITGLLLTRDGGNSNSSGKNDTETFRPGGGDMRDNWRSELYKYKVVKIEPLGVAPTKCRRRVVQ

SEQ ID NO: 51 - protein, 20 aa, synthetic construct
GGSGGGGSGGGGSGGGGSGG

SEQ ID NO: 52 - protein, 153 aa, synthetic construct
AVGIGAVFLGFLGAAGSTMGAASLTLTVQARQLLSGIVQQQNNLLRAIEAQQHLLQLTVWGIKQLQARVLAVERYLRDQQLLGIWGCSGKLICCTNVPWNASWSNKSLDAIWDNMTWMQWEKEIDNYTDFIYRLLEESQNQQEKNEQDLLELD

SEQ ID NO: 53 - protein, 709 aa, synthetic construct
VGNLWVTVYYGVPVWKEAKTTLFCASDAKAYEKEVHNVWATHACVPTDPNPQEMVLENVTENFNMWKNDMAEQMHEDVISLWDQSLKPCVKLTPLCVTLECRNANATKDNGTKGNEQVKITSNREEIKNCSFNTTTEIRDKKQKVYALFYRLDLVPLGEEKRNDSNSSSNYILINCNTSAIKQACPKVSFDPIPIHYCAPAGYAILKCNNKTFNGTGPCNNVSTVQCTHGIKPVVSTQLLLNGSLAEGEIIIRSKNLTDNAKTIIVHLNQSVEIKCIRPNNNTRKSIRIGPGQTFYATGDIIGDIRQAYCTINRTEWNNTLQGVSKKLAEHFPDKTINFTSPPAGGDLEITTHSFNCRGEFFYCNTSALFNSTYVNSTLFYRPDGNSSTDIIILCRIKQIINMWQEVGQAMYNPPIAGNITCRSNITGLLLLRDGGTGNTTDNKTEIFRPGGGDMRDNWRSELYKYKVVEIKPLGIAPTTCKRRVVEGGSGGGGSGGGGSGGGGSGGAVGIGAVFLGFLGAAGSTMGAASITLTVQARQLLSGIVQQQSNLLRAIEAQQHLLQLTVWGIKQLQTRVLAIERYLKDQQLLGIWGCSGKLICCTAVPWNSSWSNRSQEYIWGNMTWMQWDREINNYSDTIYRLLEESQNQQEKNEKDLLALDGSLEVLFQGPGSGYIPEAPRDGQAYVRKDGEWVLLSTFLHHHHHHHHHH

SEQ ID NO: 54 - protein, 487 aa, synthetic construct
VGNLWVTVYYGVPVWKEAKTTLFCASDAKAYEKEVHNVWATHACVPTDPNPQEMVLENVTENFNMWKNDMAEQMHEDVISLWDQSLKPCVKLTPLCVTLECRNANATKDNGTKGNEQVKITSNREEIKNCSFNTTTEIRDKKQKVYALFYRLDLVPLGEEKRNDSNSSSNYILINCNTSAIKQACPKVSFDPIPIHYCAPAGYAILKCNNKTFNGTGPCNNVSTVQCTHGIKPVVSTQLLLNGSLAEGEIIIRSKNLTDNAKTIIVHLNQSVEIKCIRPNNNTRKSIRIGPGQTFYATGDIIGDIRQAYCTINRTEWNNTLQGVSKKLAEHFPDKTINFTSPPAGGDLEITTHSFNCRGEFFYCNTSALFNSTYVNSTLFYRPDGNSSTDIIILCRIKQIINMWQEVGQAMYNPPIAGNITCRSNITGLLLLRDGGTGNTTDNKTEIFRPGGGDMRDNWRSELYKYKVVEIKPLGIAPTTCKRRVVE

SEQ ID NO: 55 - protein, 20 aa, synthetic construct
GGSGGGGSGGGGSGGGGSGG

SEQ ID NO: 56 - protein, 153 aa, synthetic construct
AVGIGAVFLGFLGAAGSTMGAASITLTVQARQLLSGIVQQQSNLLRAIEAQQHLLQLTVWGIKQLQTRVLAIERYLKDQQLLGIWGCSGKLICCTAVPWNSSWSNRSQEYIWGNMTWMQWDREINNYSDTIYRLLEESQNQQEKNEKDLLALD

SEQ ID NO: 57 - protein, 705 aa, synthetic construct
LGNLWVTVYYGVPVWKEAKATLFCASDAKVYEKEIHNVWATHACVPTDPNPQEMVLANVTENFNMWKNDMVDQMQEDIISLWDQSLKPCVKLTPLCVTLNCSEVTNSSITTNSSSTTNSSINANDSMSGEMRNCSFNTTTEIRDKKRKEHALFYRLDLVPLEDNNTTYRLINCNTSTVTQACPKVTFDPIPIHYCAPAGYAILKCNNITFNGTGPCTNVSTVQCTHGIKPVVSTQLLLNGSLAEGKVIIRSENLTNNAKTIIVQFKEAVEIMCTRPGNNTRKSVRIGPGQAFYTTNIIGDIRKAHCTISEEKWNNTLKEVSEELRKHFPNKTIEFKPSSGGDLEITTHSFNCRGEFFYCNTSNLFNRTYSSNSTNSASNATSNATITLPCRIKQIINMWQGVGQAIYAPPVAGNITCNSSITGLLLTRDGGNTNGENNDTETFRPGGGDMRDNWRSELYKYKVVEIKPLGIAPTKCKRRVVEGGSGGGGSGGGGSGGGGSGGAVGIGAVFLGFLGAAGSTMGAASITLTVQARQLLSGIVQQQSNLLRAIEAQQHMLQLTVWGIKQLQARVLAIERYLQDQQLLGMWGCSGKLICCTDVPWNLSWSNNKSVEDIWNNMTWMQWDKEINNYTQTIYWLLGESQIQQEKNEKDLLELDGSLEVLFQGPGSGYIPEAPRDGQAYVRKDGEWVLLSTFLHHHHHHHHHH

SEQ ID NO: 58 - protein, 482 aa, synthetic construct
LGNLWVTVYYGVPVWKEAKATLFCASDAKVYEKEIHNVWATHACVPTDPNPQEMVLANVTENFNMWKNDMVDQMQEDIISLWDQSLKPCVKLTPLCVTLNCSEVTNSSITTNSSSTTNSSINANDSMSGEMRNCSFNTTTEIRDKKRKEHALFYRLDLVPLEDNNTTYRLINCNTSTVTQACPKVTFDPIPIHYCAPAGYAILKCNNITFNGTGPCTNVSTVQCTHGIKPVVSTQLLLNGSLAEGKVIIRSENLTNNAKTIIVQFKEAVEIMCTRPGNNTRKSVRIGPGQAFYTTNIIGDIRKAHCTISEEKWNNTLKEVSEELRKHFPNKTIEFKPSSGGDLEITTHSFNCRGEFFYCNTSNLFNRTYSSNSTNSASNATSNATITLPCRIKQIINMWQGVGQAIYAPPVAGNITCNSSITGLLLTRDGGNTNGENNDTETFRPGGGDMRDNWRSELYKYKVVEIKPLGIAPTKCKRRVVE

SEQ ID NO: 59 - protein, 20 aa, synthetic construct
GGSGGGGSGGGGSGGGGSGG

SEQ ID NO: 60 - protein, 154 aa, synthetic construct
AVGIGAVFLGFLGAAGSTMGAASITLTVQARQLLSGIVQQQSNLLRAIEAQQHMLQLTVWGIKQLQARVLAIERYLQDQQLLGMWGCSGKLICCTDVPWNLSWSNNKSVEDIWNNMTWMQWDKEINNYTQTIYWLLGESQIQQEKNEKDLLELD

SEQ ID NO: 61 - protein, 698 aa, synthetic construct
SENLWATVYYGVPVWEDATTPLFCASDAKAYSPEKHNVWATHACVPTDPNPQEISLENITENFNMWRNDMVEQMHEDIISLWDESLKPCVKLTPLCVTLDCSNVTRSNIGNSTGNNNTVRNSTVNNNQEMKNCSFNITTELRDKTKKEYALFYKLDIVPLNSSNNNDSSYRLINCNVSTIKQACPKVSFEPIPIHYCAPAGFAILKCRDKNFKGTGACKNVSTVQCTHGIKPVVSTQLLLNGSLAEGEIMIRSENITDNTKIIIVQLNKSIEINCTRPGNNTRRSIRIGPGQSFYATGDIIGDIRQAHCTVNKRNWTEMLQGVRTQLKDLLENKNISFNSSTGGDLEITTHSFNCRGEFFYCDTSGLFNTTLLTTNSTDNETITLSCKIKQIVRMWQRVGQAMYAPPIRGNITCISNITGLLLTRDGGQTNSSNETFRPGGGNMRDNWRSELYKYKIVKIKPLGVAPTRCRRRVVEGGSGGGGSGGGGSGGGGSGGAVGLGALLLGFLGTAGSTMGAASITLTVQARQLLSGIVQQQSNLLRAIEAQQHLLQLTVWGIKQLQARLLAVERYLKDQQLLGIWGCSGKLICCTNVPWNTSWSNKSFEEIWDNMTWIQWDREVSKYTQEIYNLIEESQNQQERNEQDLLALDGSLEVLFQGPGSGYIPEAPRDGQAYVRKDGEWVLLSTFLHHHHHHHHHH

SEQ ID NO: 62 - protein, 476 aa, synthetic construct
SENLWATVYYGVPVWEDATTPLFCASDAKAYSPEKHNVWATHACVPTDPNPQEISLENITENFNMWRNDMVEQMHEDIISLWDESLKPCVKLTPLCVTLDCSNVTRSNIGNSTGNNNTVRNSTVNNNQEMKNCSFNITTELRDKTKKEYALFYKLDIVPLNSSNNNDSSYRLINCNVSTIKQACPKVSFEPIPIHYCAPAGFAILKCRDKNFKGTGACKNVSTVQCTHGIKPVVSTQLLLNGSLAEGEIMIRSENITDNTKIIIVQLNKSIEINCTRPGNNTRRSIRIGPGQSFYATGDIIGDIRQAHCTVNKRNWTEMLQGVRTQLKDLLENKNISFNSSTGGDLEITTHSFNCRGEFFYCDTSGLFNTTLLTTNSTDNETITLSCKIKQIVRMWQRVGQAMYAPPIRGNITCISNITGLLLTRDGGQTNSSNETFRPGGGNMRDNWRSELYKYKIVKIKPLGVAPTRCRRRVVE

SEQ ID NO: 63 - protein, 20 aa, synthetic construct
GGSGGGGSGGGGSGGGGSGG

SEQ ID NO: 64 - protein, 153 aa, synthetic construct
AVGLGALLLGFLGTAGSTMGAASITLTVQARQLLSGIVQQQSNLLRAIEAQQHLLQLTVWGIKQLQARLLAVERYLKDQQLLGIWGCSGKLICCTNVPWNTSWSNKSFEEIWDNMTWIQWDREVSKYTQEIYNLIEESQNQQERNEQDLLALD

SEQ ID NO: 65 - protein, 706 aa, synthetic construct
TDQLWVTVYYGVPVWKEATTTLFCASDAKGYDTEVHNVWATHACVPTDPNPQEVVMGNVTENFNMWKNSMVDQMHEDIISLWDQSLQPCVKLTPLCVTLKCNDTWGNATQTNNSRVVTTPTTDSKLGKREMTNCSFNITSNIRDRVQEEHALFYKFDVVPIKEDDNNNNTRYRLIHCNTSVITQACPKVSFEPIPIHYCAPAGFAILKCNDNKFNGSGPCRNVSTVQCTHGIRPVVSTQLLLNGSLAEEEIVIRSSNFTDNAKTIIVQLNKSVEITCIRPGNNTRKSIPIGPGRAFFATGDVIGDIRKAHCTLNRTDWRNTLKQIATKLRRQFENKTISFQKSSGGDPEIVMHSFNCGGEFFYCNTTQLFDSNWTLSDITKGSNTTEGNSTHITLPCRIKQIINMWQEVGKAMYAPPIRGNITCISNITGLLLVRDGGNNNETEIFRPGGGDMRDNWRSELYKYKVIKIEPLGIAPTKCKRRVVQGGSGGGGSGGGGSGGGGSGGAALGALFLGFLGAAGSTMGAASLTLTVQARLLLSGIVQQQNNLLRAIEAQQHLLQLTVWGIKQLQARVLAVERYLRDQQLLGIWGCSGRLVCCTDVKWNTTWSNKSLEEIWNNMTWMEWEREIENYTSEIYTLIEKSQNQQEKNEQKLLELDGSLEVLFQGPGSGYIPEAPRDGQAYVRKDGEWVLLSTFLHHHHHHHHHH

SEQ ID NO: 66 - protein, 485 aa, synthetic construct
TDQLWVTVYYGVPVWKEATTTLFCASDAKGYDTEVHNVWATHACVPTDPNPQEVVMGNVTENFNMWKNSMVDQMHEDIISLWDQSLQPCVKLTPLCVTLKCNDTWGNATQTNNSRVVTTPTTDSKLGKREMTNCSFNITSNIRDRVQEEHALFYKFDVVPIKEDDNNNNTRYRLIHCNTSVITQACPKVSFEPIPIHYCAPAGFAILKCNDNKFNGSGPCRNVSTVQCTHGIRPVVSTQLLLNGSLAEEEIVIRSSNFTDNAKTIIVQLNKSVEITCIRPGNNTRKSIPIGPGRAFFATGDVIGDIRKAHCTLNRTDWRNTLKQIATKLRRQFENKTISFQKSSGGDPEIVMHSFNCGGEFFYCNTTQLFDSNWTLSDITKGSNTTEGNSTHITLPCRIKQIINMWQEVGKAMYAPPIRGNITCISNITGLLLVRDGGNNNETEIFRPGGGDMRDNWRSELYKYKVIKIEPLGIAPTKCKRRVVQ

SEQ ID NO: 67 - protein, 20 aa, synthetic construct
GGSGGGGSGGGGSGGGGSGG

SEQ ID NO: 68 - protein, 152 aa, synthetic construct
AALGALFLGFLGAAGSTMGAASLTLTVQARLLLSGIVQQQNNLLRAIEAQQHLLQLTVWGIKQLQARVLAVERYLRDQQLLGIWGCSGRLVCCTDVKWNTTWSNKSLEEIWNNMTWMEWEREIENYTSEIYTLIEKSQNQQEKNEQKLLELD

SEQ ID NO: 69 - protein, 702 aa, synthetic construct
EEKLWVTVHYGVPVWKEATTTLFCASDAKAYKTEAHNVWATHACVPTDPNPREVPLENVTENFNMWKNNMVEQMHEDIISLWDQSLKPCVKLTPLCVILNCSNLNNSTNITSSDQGNLDMREEIKNCSFNITTGIGKKVRKDYAIFNRIDIVPIDDNDSKNNSSNNTSYMLRSCDTSVITQACPKVTFEPIPIHYCTPAGYAILKCNDKKFNGTGPCKNVSTVQCTHGIKPVVSTQLLLNGSLAEEEVIIRSENITDNGKNIIVQLNETVKINCTRPNNNTRKSIHMGWGRAFYATGAIIGDIRQAHCNLSRAEWNKTLEKIAIKLKERVNKTKIIFNQSSGGDSEIEMHSFNCGGEFFYCNTTQLFNSTWNGTHLSNSIGNETITLPCRIKQIINRWQEVGKAMYAPPISGQISCSSNITGLILTRDGSNDNSSTNETFRPGGGNMKDNWRSELYKYKVVEIEPVGLAPTKCKRRVVQGGSGGGGSGGGGSGGGGSGGAVGTLGAMFLGFLGTAGSTMGAASLTLTVQARQLMSGIVQQQNNLLRAIEAQQHMLQLTVWGIKQLQARVLAVERYLRDQQLLGIWGCSGKLICCTAVPWNTSWSNKYLSYIWNNMTWMQWEREIDNYTNLIYNLLEESQNQQEKNEQELLELDGSLEVLFQGPGSGYIPEAPRDGQAYVRKDGEWVLLSTFLHHHHHHHHHH

SEQ ID NO: 70 - protein, 479 aa, synthetic construct
EEKLWVTVHYGVPVWKEATTTLFCASDAKAYKTEAHNVWATHACVPTDPNPREVPLENVTENFNMWKNNMVEQMHEDIISLWDQSLKPCVKLTPLCVILNCSNLNNSTNITSSDQGNLDMREEIKNCSFNITTGIGKKVRKDYAIFNRIDIVPIDDNDSKNNSSNNTSYMLRSCDTSVITQACPKVTFEPIPIHYCTPAGYAILKCNDKKFNGTGPCKNVSTVQCTHGIKPVVSTQLLLNGSLAEEEVIIRSENITDNGKNIIVQLNETVKINCTRPNNNTRKSIHMGWGRAFYATGAIIGDIRQAHCNLSRAEWNKTLEKIAIKLKERVNKTKIIFNQSSGGDSEIEMHSFNCGGEFFYCNTTQLFNSTWNGTHLSNSIGNETITLPCRIKQIINRWQEVGKAMYAPPISGQISCSSNITGLILTRDGSNDNSSTNETFRPGGGNMKDNWRSELYKYKVVEIEPVGLAPTKCKRRVVQ

SEQ ID NO: 71 - protein, 20 aa, synthetic construct
GGSGGGGSGGGGSGGGGSGG

SEQ ID NO: 72 - protein, 154 aa, synthetic construct
AVGTLGAMFLGFLGTAGSTMGAASLTLTVQARQLMSGIVQQQNNLLRAIEAQQHMLQLTVWGIKQLQARVLAVERYLRDQQLLGIWGCSGKLICCTAVPWNTSWSNKYLSYIWNNMTWMQWEREIDNYTNLIYNLLEESQNQQEKNEQELLELD

SEQ ID NO: 73 - protein, 707 aa, synthetic construct
ASQLWVTVYYGVPVWKEATTTLFCASNAKAYDPEVHNVWATHACVPTDPSPQEVKLNVTENFNMWKNDMVEQMHEDIISIWDQSLTPCVKLTPLCVTLNCTNNITYNNNINTTTSNNNTNNTTNDWEKMEPGEIKNCSFNITTNVRDKVQKTYALFNSLDVVPIDSDNSSYMIISCNTSATTQACPKVSFEPIPIHFCAPAGFAILKCNSKTFNGTGPCKNVSIVQCTHGIRPVVSTQLLLNGSLAEEDVIIRSENFTNNAKTILVQLNESVIINCTRPNNNTRKGIHMGPGRTIYATGNIIGDIRKAYCNLNKTDWERALKRIGIKLREQFENKTIAFNSSSGGDPEIVMHSFNCRGEFFYCNSTPLFHYTWDGTNGTWGNNASKGNITLHCRIKQIVNMWQRVGRAMYAPPISGPISCSSNITGLLLTRDGGGINETNTTETFRPGGGDMKDNWRSELYKYKVVQIEPIGIAPTKCKRRVVQGGSGGGGSGGGGSGGGGSGGAVGVFGAMFLGFLGAAGSTMGAASITLTAQARQLLSGIVQQQSNLLRAIEAQQHMLQLTVWGIKQLQARVLAVERYLKDQQLLGIWGCSGKLICCTTVPWNASWSNKSVNEIWDNMTWMQWEREIDNYTNQIYNLLEKSQNQQEKNEQELLELDGSLEVLFQGPGSGYIPEAPRDGQAYVRKDGEWVLLSTFLHHHHHHHHHH

SEQ ID NO: 74 - protein, 484 aa, synthetic construct
ASQLWVTVYYGVPVWKEATTTLFCASNAKAYDPEVHNVWATHACVPTDPSPQEVKLNVTENFNMWKNDMVEQMHEDIISIWDQSLTPCVKLTPLCVTLNCTNNITYNNNINTTTSNNNTNNTTNDWEKMEPGEIKNCSFNITTNVRDKVQKTYALFNSLDVVPIDSDNSSYMIISCNTSATTQACPKVSFEPIPIHFCAPAGFAILKCNSKTFNGTGPCKNVSIVQCTHGIRPVVSTQLLLNGSLAEEDVIIRSENFTNNAKTILVQLNESVIINCTRPNNNTRKGIHMGPGRTIYATGNIIGDIRKAYCNLNKTDWERALKRIGIKLREQFENKTIAFNSSSGGDPEIVMHSFNCRGEFFYCNSTPLFHYTWDGTNGTWGNNASKGNITLHCRIKQIVNMWQRVGRAMYAPPISGPISCSSNITGLLLTRDGGGINETNTTETFRPGGGDMKDNWRSELYKYKVVQIEPIGIAPTKCKRRVVQ

SEQ ID NO: 75 - protein, 20 aa, synthetic construct
GGSGGGGSGGGGSGGGGSGG

SEQ ID NO: 76 - protein, 154 aa, synthetic construct
AVGVFGAMFLGFLGAAGSTMGAASITLTAQARQLLSGIVQQQSNLLRAIEAQQHMLQLTVWGIKQLQARVLAVERYLKDQQLLGIWGCSGKLICCTTVPWNASWSNKSVNEIWDNMTWMQWEREIDNYTNQIYNLLEKSQNQQEKNEQELLELD

SEQ ID NO: 77 - protein, 139 aa, synthetic construct
MRTLWIMAVLLLGVEGNLYQFEKLIQKIVGRSGVLWYSAYGCYCGWGGQGRPQDATDRCCFVHDCCYNKVTSCNPKLDIYTYSVKNRDVVCGGTNPCKKQICECDRAAAICFRDNKDTYDSKKYWKYPKKNCKEESEPC

SEQ ID NO: 78 - protein, 137 aa, synthetic construct
MRLSLTDNRLFSDRSKSPFPDFSPAEAINGKNAITSYGSYGCNCGWGHRGQPKDATDRCCFVHKCCYKKLTDCNHKTDRYSYSWKNKAIICEEKNPCLKEMCECDKAVAICLRENLDTYNKKYKAYFKLKCKKPDTC

SEQ ID NO: 79 - protein, 132 aa, synthetic construct
MAVLLLGVEGNLFQFEKLIKKMTGKSGMLCYSAYGCYCGWGGQGRPKDATDRCCFVHDCCYGKVTGCDPKLDSYTYSVENGDVVCGGNDPCKKEICECDRAAAICFRDNKVTYDNKYWRFPPQNCKEESEPC

SEQ ID NO: 80 - protein, 138 aa, synthetic construct
MRTLWIVAVWLIGVEGSVIEFGTMIIEETGRSPFPFYTSYGCYCGLGGKGKPKDDTDRCCFVHDCCYGGMPDCSPKTDIYRYHRENGEIICESGTSCEKRICECDKAAAVCFRENLKTYKNKYMVYPDSLCKEESEKC

SEQ ID NO: 81 - protein, 121 aa, synthetic construct
NLVQFKTLIMKIAGRSVVYKYFYGCYCGWGGIGQPRDATDRCCFVHDCCYGKVTNCNPKTATYSYTEENGALVCGGDDPCKKQVCECDRVAAMCFRDNKDTYDNKYWFLPPKNCQEDSEPC

SEQ ID NO: 82 - protein, 138 aa, synthetic construct
MRTLWIMAVLLVGVEGSLIEFAKMILEETKRLPFPYYTTYGCYCGWGGQGQPKDATDRCCFVHDCCYGKLSNCKPKTDRYSYSRKSGVIICGEGTPCEKQICECDKAAAVCFRENLRTYKKRYMAYPDLLCKKPAEKC

SEQ ID NO: 83 - protein, 138 aa, synthetic construct
MRTLWIMAVLLVGVEGNLWQLGKMILLETGKIPAKSYAAYGCNCGVLGRGKPKDATDRCCYVHKCCYKKLTGCDPKKDRYSYSWKDKTIVCGENNSCLKELCECDKAVAICLRENLDTYNKKYRYNYLKPFCKKAEPC

SEQ ID NO: 84 - protein, 124 aa, synthetic construct
DLWQFGQMMNDVMREYVVFNYLYYGCYCGWGGIGKPRDATDRCCFVHDCCYGKVTGCNPKTDSYTYTYSEENGDVVCGGDDLCKKQICECDRVAATCFRDNKDTYDTKYWLYGAKNCQEESEPC

SEQ ID NO: 85 - protein, 156 aa, synthetic construct
MACSLGGSSPVLLLCVLVFASANLIQFGHIIEHLTGRHPLIYNGYGCYCGLGGSRQPVDATDWCCQVHDCCYQALSRRHCKPKMEKYFYSVRKDTVTCGGETECRRQTCECDKAAALCFRHSKFQDQYIGYHNRLCEGPTPPCQGVCPCWAPTKGG

SEQ ID NO: 86 - protein, 138 aa, synthetic construct
MRALWILAVLLLGVEGSLVEFETLIMKIAGRSGVWYYSSYGCYCGAGGQGWPQDASDRCCFEHDCCYAKLTGCDPTTDVYTYRQEDGEIVCGGDDPCGTQICECDKAAAICFRDSMDTYDHKYWRFSLENCQGESQPC

SEQ ID NO: 87 - protein, 138 aa, synthetic construct
  VARIANT 53: note = Any amino acid
  VARIANT 54: note = Any amino acid
GCPLGKAPSRPRRGILQLAGMIQCTTGRTPLAYIRYGCYCGWGGRGWPKDQVXXCCFKHDCCYGRAEEHSCAPKTWWYPWECQDGKAKCDDIEDKCQKMACECDRSAAKCLAKAPYNMTYLFWPDTQCGEKGPTCPDD

SEQ ID NO: 88 - protein, 138 aa, synthetic construct
MRTLWILAVCLIGVEGNLFQFARMINGKLGAFSVWNYISYGCYCGWGGQGTPKDATDRCCFVHDCCYGGVKGCNPKLAIYSYSFQRGNIVCGRNNGCLRTICECDRVAANCFHQNKNTYNKRYKFLSSYYCRQTSEKC

SEQ ID NO: 89 - protein, 138 aa, synthetic construct
MRALWIVAVLLVGVEGSLFELGKMIWQETGKNPVKNYGLYGCNCGVGGRGEPLDATDRCCFVHKCCYKKLTDCDSKKDRYSYKWKNKAIVCGKNQPCMQEMCECDKAFAICLRENLDTYNKSFRYHLKPLCKKTSEQC

SEQ ID NO: 90 - protein, 138 aa, synthetic construct
MRALWIVAVWLIGVEGSVVELGKMIIQETGKSPFPSYTSYGCFCGGGEKGTPKDATDRCCFVHSCCYDKLPDCSPKTDRYKYQRENGEIICENSTSCKKRICECDKAVAVCLRENLQTYNKKYTYYPNFLCKGEPEKC

SEQ ID NO: 91 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0027196 A1
(43) Pub. Date: Jan. 29, 2026

(54) LOW DOSE VACCINE COMPOSITIONS

(71) Applicant: Centivax, Inc., South San Francisco, CA (US)

(72) Inventors: Jacob Glanville, San Francisco, CA (US); Rishi Bedi, Menlo Park, CA (US); Mark Bellin, San Francisco, CA (US); Sangil Kim, Redwood City, CA (US); David Tsao, Sunnyvale, CA (US)

(21) Appl. No.: 19/200,318

(22) Filed: May 6, 2025

Related U.S. Application Data
(63) Continuation of application No. PCT/US2023/079255, filed on Nov. 9, 2023.
(60) Provisional application No. 63/383,034, filed on Nov. 9, 2022.

(51) Int. Cl. A61K 39/145; A61K 39/00; A61P 31/16; C12N 7/00

(52) U.S. Cl. CPC A61K 39/145; A61P 31/16; C12N 7/00; A61K 2039/5258; A61K 2039/53; A61K 2039/545; A61K 2039/55; C12N 2760/16123; C12N 2760/16134

(57) ABSTRACT

The present disclosure provides a vaccine composition comprising six or more homologous distinct antigen components. Any two of the six or more homologous distinct antigen components may share less than 98% or 95% sequence identity. The homologous distinct antigen components may comprise proteins. An amount of a protein in a dose of said human adult vaccine composition may be about 600 nanograms (ng) to about 3 micrograms (μg). The homologous distinct antigen components may comprise a plurality of RNA. An amount of an RNA of said plurality of RNA in a dose of said vaccine composition may be about 1 ng to about 5 μg per dose. The homologous distinct antigen components may comprise a plurality of proteins displayed on heterologous viral-like particles (VLPs). An amount of a protein displayed on a heterologous VLP of the plurality of proteins displayed on heterologous VLPs in a dose of the vaccine composition may be about 1 ng to about 5 μg per dose.

### CROSS-REFERENCE

This application is a continuation application of International Patent Application No. PCT/US2023/079255, filed Nov. 9, 2023, which claims the benefit of U.S. Provisional Application No. 63/383,034, filed Nov. 9, 2022, each of which is entirely incorporated herein by reference.

### SEQUENCE LISTING

The instant application contains a Sequence Listing which has been submitted electronically in XML format and is hereby incorporated by reference in its entirety. Said XML copy, created on Mar. 11, 2024, is named 60333-726_601_SL.xml and is 130,134 bytes in size.

### BACKGROUND

Vaccines may comprise multiple homologous antigen components, especially for pathogens that belong to diverse phylogenetic families (e.g., influenza). For some antigen components, there may be a minimum dose of that antigen component necessary to elicit an optimal immune response against that antigen component alone.

### SUMMARY

The present disclosure provides a vaccine composition comprising: a plurality of RNAs that collectively encode for six or more homologous distinct antigen components, wherein any two of said six or more homologous distinct antigen components share less than 98% sequence identity, wherein each RNA that encodes for a distinct antigen component is present in the composition in an amount from 1 ng to 5 micrograms per dose; a plurality of viral-like particles that collectively display at least six or more homologous distinct antigen components, wherein any two of said six or more homologous antigen components share less than 98% sequence identity, wherein each distinct antigen component is present in the composition in an amount from 1 ng to 5 micrograms; or at least six or more homologous distinct antigen components, wherein any two of said six or more homologous antigen components share less than 98% sequence identity, wherein each distinct antigen component of said six or more homologous distinct antigen components is present in the composition in an amount from 550 ng to 5 micrograms. In some embodiments, said vaccine composition is for the prevention of influenza. In some embodiments, said vaccine composition is for the prevention of HIV. In some embodiments, each RNA that encodes for a distinct antigen component is present in the composition in an amount from 1 ng to 2.5 micrograms per dose. In some embodiments, each RNA that encodes for a distinct antigen component is present in the composition in an amount from 1 ng to 1.5 micrograms per dose. In some embodiments, each RNA that encodes for a distinct antigen component is present in the composition in an amount from 1 ng to 1 microgram per dose. In some embodiments, each RNA that encodes for a distinct antigen component is present in the composition in an amount of about 0.5 micrograms per dose. In some embodiments, each RNA that encodes for a distinct antigen component is present in the composition in an amount of 1 microgram per dose. In some embodiments, each RNA that encodes for a distinct antigen component is present in the composition in an amount of 2 micrograms per dose. In some embodiments, each RNA that encodes for a distinct antigen component is present in the composition in an amount from 1 ng to 4 micrograms per dose. In some embodiments, each RNA that encodes for a distinct antigen component is present in the composition in an amount greater than 10 nanograms per dose. In some embodiments, each RNA that encodes for a distinct antigen component is present in the composition in an amount greater than 100 nanograms per dose. In some embodiments, each RNA that encodes for a distinct antigen component is present in the composition in an amount greater than 250 nanograms per dose. In some embodiments, each RNA that encodes for a distinct antigen component is present in the composition in an amount greater than 500 nanograms per dose. In some embodiments, each distinct antigen component displayed on VLPs is present in the composition in an amount from 1 ng to 2.5 micrograms per dose. In some embodiments, each distinct antigen component displayed on VLPs is present in the composition in an amount from 1 ng to 1.5 micrograms per dose. In some embodiments, each distinct antigen component displayed on VLPs is present in the composition in an amount from 1 ng to 1 microgram per dose. In some embodiments, each distinct antigen component displayed on VLPs is present in the composition in an amount of about 0.5 micrograms per dose. In some embodiments, each distinct antigen component of said six or more homologous distinct antigen components is present in the composition in an amount from 550 ng to 2.5 micrograms per dose. In some embodiments, each distinct antigen component of said six or more homologous distinct antigen components is present in the composition in an amount from 550 ng to 1.5 micrograms per dose. In some embodiments, each distinct antigen component of said six or more homologous distinct antigen components is present in the composition in an amount from 550 ng to 1 microgram per dose. In some embodiments, each of the at least six or more homologous distinct antigen components is present in the composition in an amount from 550 ng to 3000 ng. In some embodiments, each of the at least six or more homologous distinct antigen components is present in the composition in an amount from 600 ng to 3000 ng. In some embodiments, each of the at least six or more homologous distinct antigen components is present in the composition in an amount from 750 ng to 3000 ng. In some embodiments, each of the at least six or more homologous distinct antigen components is present in the composition in an amount from 1000 ng to 3000 ng. In some embodiments, each of the at least six or more homologous distinct antigen components is present in the composition in an amount from 750 ng to 2000 ng. In some embodiments, said vaccine composition is for administration in a human subject. In some embodiments, said vaccine composition is for administration in an adult who is 18 years of age or older. In some embodiments, said vaccine composition is for administration in an adult who is 25 years of age or older. In some embodiments, said vaccine composition is for administration in an adult who is 50 years of age or older. In some embodiments, said vaccine composition is for administration in an adult who is 75 years of age or older. In some embodiments, said vaccine composition is for administration in a child between the ages of about 1 day old and about 18 years old. In some embodiments, said vaccine composition is for administration in a child between the ages of about 1 day old and about 5 years old. In some embodiments, said vaccine composition is for administration in a child between the ages of about 5 years old and about 18 years old. In some embodiments, said vaccine composition is for administration in an animal. In some embodiments, the animal is a livestock animal. In some embodiments, the livestock animal is a cow, a bull, an alpaca, a llama, a sheep, a pig, or a bird. In some embodiments, the animal is a domesticated animal. In some embodiments, the domesticated animal is a primate. In some embodiments, the six or more homologous distinct antigen components comprise an antigen from a virus, a bacterium, a fungus, a prion, or a plant. In some embodiments, the homologous distinct antigen components comprise a component of a virus. In some embodiments, the component of the virus is a protein comprising a receptor-binding domain. In some embodiments, the component of the virus is a receptor-binding domain. In some embodiments, at least two homologous distinct antigen components of said six or more homologous distinct antigen components comprise receptor binding domains for a cell surface protein. In some embodiments, said cell surface protein is mammalian. In some embodiments, said receptor binding domains of said any two homologous distinct antigen components of said six or more homologous distinct antigen components share less than 99% sequence identity. In some embodiments, said receptor binding domains of said any two homologous distinct antigen components of said six or more homologous distinct antigen components share less than 98% sequence identity. In some embodiments, said receptor binding domains of said any two homologous distinct antigen components of said six or more homologous distinct antigen components share less than 95% sequence identity. In some embodiments, said receptor binding domains of said any two homologous distinct antigen components of said six or more homologous distinct antigen components share less than 95% sequence identity. In some embodiments, said receptor binding domains of said any two homologous distinct antigen components of said six or more homologous distinct antigen components share less than 90% sequence identity. In some embodiments, said receptor binding domains of said any two homologous distinct antigen components of said six or more homologous distinct antigen components share less than 85% sequence identity. In some embodiments, said receptor binding domains of said any two homologous distinct antigen components of said six or more homologous distinct antigen components share less than 80% sequence identity. In some embodiments, said receptor binding domains of said any two homologous distinct antigen components of said six or more homologous distinct antigen components share less than 75% sequence identity. In some embodiments, said receptor binding domains of said any two homologous distinct antigen components of said six or more homologous distinct antigen components share less than 70% sequence identity. In some embodiments, an administration of said vaccine composition reduces a dominant population of immunogenic single variant epitopes corresponding to said homologous distinct antigen components. In some embodiments, any 2 of said six or more homologous distinct antigen components share less than about 98% sequence identity. In some embodiments, any 2 of said six or more homologous distinct antigen components share less than about 97% sequence identity. In some embodiments, any 2 of said six or more homologous distinct antigen components share less than about 96% sequence identity. In some embodiments, any 2 of said six or more homologous distinct antigen components share less than about 95% sequence identity. In some embodiments, any 2 of said six or more homologous distinct antigen components share less than about 96% sequence identity. In some embodiments, any 2 of said six or more homologous distinct antigen components share less than about 95% sequence identity. In some embodiments, any 2 of said six or more homologous distinct antigen components share less than about 90% sequence identity. In some embodiments, any 2 of said six or more homologous distinct antigen components share less than about 85% sequence identity. In some embodiments, any 2 of said six or more homologous distinct antigen components share less than about 80% sequence identity. In some embodiments, any 2 of said six or more homologous distinct antigen components share less than about 75% sequence identity. In some embodiments, any 2 of said six or more homologous distinct antigen components share less than about 70% sequence identity. In some embodiments, said six or more homologous distinct antigen components comprise 7 or more homologous distinct antigen components. In some embodiments, said six or more homologous distinct antigen components comprise 10 or more homologous distinct antigen components. In some embodiments, said six or more homologous distinct antigen components comprise 15 or more homologous distinct antigen components. In some embodiments, said six or more homologous distinct antigen components comprise 20 or more homologous distinct antigen components. In some embodiments, said six or more homologous distinct antigen components comprise 25 or more homologous distinct antigen components. In some embodiments, said six or more homologous distinct antigen components comprise 30 or more homologous distinct antigen components. In some embodiments, any 2 of said 6 or more homologous distinct antigen components share at least 30% sequence identity. In some embodiments, any 2 of said 6 or more homologous distinct antigen components share at least 40% sequence identity. In some embodiments, any 2 of said 6 or more homologous distinct antigen components share at least 50% sequence identity. In some embodiments, any 2 of said 6 or more homologous distinct antigen components share at least 60% sequence identity. In some embodiments, any 2 of said 6 or more homologous distinct antigen components share at least 70% sequence identity. In some embodiments, said vaccine composition further comprises an adjuvant. In some embodiments, said plurality of RNA are present in said vaccine composition at the same amount. In some embodiments, said plurality of RNA are present in said vaccine composition at distinct amounts. In some embodiments, a dose of an RNA of said plurality of RNA is calculated to be proportional to the phylogenetic distance between a homologous distinct antigen component of said six or more homologous distinct antigen components to another homologous distinct antigen component, wherein said another homologous distinct antigen component has the least distance to said homologous distinct antigen components out of said six or more homologous distinct antigen components. In some embodiments, a dose of an RNA of said plurality of RNAs is calculated to be proportional to an average phylogenetic distance of a homologous distinct antigen component of said six or more homologous antigen component to other homologous distinct antigen components of said six or more homologous distinct antigen components.

Another aspect of the present disclosure provides a method of inducing an immune response against an influenza pathogen in a subject comprising administering any vaccine composition as disclosed herein. In some embodiments, the method is for prophylaxis against influenza.

An aspect of the present disclosure provides a vaccine composition comprising six or more homologous distinct antigen components, wherein any two of said six or more homologous distinct antigen components share less than 95% sequence identity and wherein the homologous distinct antigen components comprise proteins, and wherein a concentration/amount of a protein in a dose of said human adult vaccine composition is about 1 nanogram (ng) to about 3 micrograms (μg); wherein the homologous distinct antigen components comprise a plurality of RNA, and wherein a concentration/amount of an RNA of said plurality of RNA in a dose of said vaccine composition is about 1 ng to about 5 μg per dose; or wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said vaccine composition is about 1 ng to about 5 μg per dose. In some embodiments, said vaccine composition is for administration in a human subject. In some embodiments, said vaccine composition is for administration in an adult who is 18 years of age or older. In some embodiments, said vaccine composition is for administration in an adult who is 25 years of age or older. In some embodiments, said vaccine composition is for administration in an adult who is 50 years of age or older. In some embodiments, said vaccine composition is for administration in an adult who is 75 years of age or older. In some embodiments, said vaccine composition is for administration in a child between the ages of about 1 day old and about 18 years old. In some embodiments, said vaccine composition is for administration in a child between the ages of about 1 day old and about 5 years old. In some embodiments, said vaccine composition is for administration in a child between the ages of about 5 years old and about 18 years old. In some embodiments, said vaccine composition is for administration in an animal. In some embodiments, the animal is a livestock animal. In some embodiments, the livestock animal is a cow, a bull, an alpaca, a llama, a sheep, a pig, or a bird. In some embodiments, the animal is a domesticated animal. In some embodiments, the domesticated animal is a primate. In some embodiments, six or more homologous distinct antigen components comprise an antigen selected from the group consisting of a virus, a bacterium, a fungus, a prion, a plant, or a combination thereof. In some embodiments, a concentration/amount of a homologous distinct antigen components component of said homologous distinct antigen components in a dose of said vaccine composition is about 1 nanogram (ng) to about 5 micrograms (μg). In some embodiments, a concentration/amount of a homologous distinct antigen components component of said homologous distinct antigen components in a dose of said vaccine composition is about 1 nanogram (ng) to about 1 microgram (μg). In some embodiments, the homologous distinct antigen components comprise a component of a virus. In some embodiments, the component of the virus is a receptor-binding domain. In some embodiments, at least two homologous distinct antigen components of said six or more homologous distinct antigen components comprise receptor binding domains for a cell surface protein. In some embodiments, said cell surface protein is mammalian. In some embodiments, said receptor binding domains of said at least two homologous distinct antigen components of said six or more homologous distinct antigen components share less than 99% sequence identity. In some embodiments, said receptor binding domains of said at least two homologous distinct antigen components of said six or more homologous distinct antigen components share less than 98% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 90% sequence identity. In some embodiments, said receptor binding domains of said at least two homologous distinct antigen components of said six or more homologous distinct antigen components share less than 85% sequence identity. In some embodiments, said receptor binding domains of said at least two homologous distinct antigen components of said six or more homologous distinct antigen components share less than 80% sequence identity. In some embodiments, said receptor binding domains of said at least two homologous distinct antigen components of said six or more homologous distinct antigen components share less than 75% sequence identity. In some embodiments, said receptor binding domains of said at least two homologous distinct antigen components of said six or more homologous distinct antigen components share less than 70% sequence identity. In some embodiments, an administration of said vaccine composition reduces a dominant population of immunogenic single variant epitopes corresponding to said homologous distinct antigen components. In some embodiments, any 2 of said six or more homologous distinct antigen components share less than about 90% sequence identity. In some embodiments, any 2 of said six or more homologous distinct antigen components share less than about 85% sequence identity. In some embodiments, any 2 of said six or more homologous distinct antigen components share less than about 80% sequence identity. In some embodiments, any 2 of said six or more homologous distinct antigen components share less than about 75% sequence identity. In some embodiments, any 2 of said six or more homologous distinct antigen components share less than about 70% sequence identity. In some embodiments, said vaccine composition comprises 7 or more homologous distinct antigen components. In some embodiments, said vaccine composition comprises 10 or more homologous distinct antigen components. In some embodiments, said vaccine composition comprises 15 or more homologous distinct antigen components. In some embodiments, said vaccine composition comprises 20 or more homologous distinct antigen components. In some embodiments, said vaccine composition comprises 25 or more homologous distinct antigen components. In some embodiments, said vaccine composition comprises 30 or more homologous distinct antigen components. In some embodiments, said vaccine composition further comprises one or more non-homologous antigen components. In some embodiments, said vaccine composition further comprises an adjuvant. In some embodiments, said six or more homologous distinct antigen components are present in said vaccine composition at the same concentration. In some embodiments, said six or more homologous distinct antigen components are present in said vaccine composition at the distinct concentrations/amounts. In some embodiments, a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components. In some embodiments, a dose of a homologous distinct antigen components of said six or more homologous distinct antigen components is calculated to be proportional to its average phylogenetic distance from the other homologous distinct antigen components of said six or more homologous distinct antigen components. In some embodiments, said vaccine composition comprises a fragment of a SARS virus. In some embodiments, said vaccine composition comprises a fragment of an influenza virus. In some embodiments, said vaccine composition comprises a fragment of an HIV virus. In some embodiments, said vaccine composition comprises a fragment of a SARS1 virus.

Another aspect of the present disclosure provides a vaccine composition for human adults comprising six or more homologous distinct antigen components, wherein two of said six or more homologous distinct antigen components share less than 95% sequence identity, and wherein the homologous distinct antigen components comprise proteins, and wherein a concentration/amount of a protein in a dose of said human adult vaccine composition is about 1 nanogram (ng) to about 3 micrograms (μg); wherein the homologous distinct antigen components comprise a plurality of RNA, and wherein a concentration/amount of an RNA of said plurality of RNA in a dose of said human adult vaccine composition is about 1 ng to about 5 μg per dose; or wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said human adult vaccine composition is about 1 ng to about 5 μg per dose. In some embodiments, said vaccine composition is for administration in an adult who is 25 years of age or older. In some embodiments, said vaccine composition is for administration in an adult who is 50 years of age or older. In some embodiments, said vaccine composition is for administration in an adult who is 75 years of age or older. In some embodiments, a concentration/amount of a homologous distinct antigen components component of said homologous distinct antigen components in a dose of said vaccine composition is about 1 nanogram (ng) to about 2 μg. In some embodiments, a concentration/amount of a homologous distinct antigen components component of said homologous distinct antigen components in a dose of said vaccine composition is about 1 nanogram (ng) to about 1 microgram (μg). In some embodiments, a concentration/amount of a homologous distinct antigen components component of said homologous distinct antigen components in a dose of said vaccine composition is about 1 nanogram (ng) to about 0.1 microgram (μg). In some embodiments, the homologous distinct antigen components comprise a component of a virus. In some embodiments, the component of the virus is a receptor-binding domain. In some embodiments, at least two homologous distinct antigen components of said six or more homologous distinct antigen components comprise receptor binding domains for a cell surface protein. In some embodiments, said cell surface protein is mammalian. In some embodiments, said receptor binding domains of said at least two homologous distinct antigen components of said six or more homologous distinct antigen components share less than 99% sequence identity. In some embodiments, said receptor binding domains of said at least two homologous distinct antigen components of said six or more homologous distinct antigen components share less than 98% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 90% sequence identity. In some embodiments, said receptor binding domains of said at least two homologous distinct antigen components of said six or more homologous distinct antigen components share less than 85% sequence identity. In some embodiments, said receptor binding domains of said at least two homologous distinct antigen components of said six or more homologous distinct antigen components share less than 80% sequence identity. In some embodiments, said receptor binding domains of said at least two homologous distinct antigen components of said six or more homologous distinct antigen components share less than 75% sequence identity. In some embodiments, said receptor binding domains of said at least two homologous distinct antigen components of said six or more homologous distinct antigen components share less than 70% sequence identity. In some embodiments, an administration of said vaccine composition reduces a dominant population of immunogenic single variant epitopes corresponding to said homologous distinct antigen components. In some embodiments, any 2 of said six or more homologous distinct antigen components share less than about 90% sequence identity. In some embodiments, any 2 of said six or more homologous distinct antigen components share less than about 85% sequence identity. In some embodiments, any 2 of said six or more homologous distinct antigen components share less than about 80% sequence identity. In some embodiments, any 2 of said six or more homologous distinct antigen components share less than about 75% sequence identity. In some embodiments, any 2 of said six or more homologous distinct antigen components share less than about 70% sequence identity. In some embodiments, said vaccine composition comprises 7 or more homologous distinct antigen components. In some embodiments, said vaccine composition comprises 10 or more homologous distinct antigen components. In some embodiments, said vaccine composition comprises 15 or more homologous distinct antigen components. In some embodiments, said vaccine composition comprises 20 or more homologous distinct antigen components. In some embodiments, said vaccine composition comprises 25 or more homologous distinct antigen components. In some embodiments, said vaccine composition comprises 30 or more homologous distinct antigen components. In some embodiments, any 2 of said six or more homologous distinct antigen components share at least about 25% sequence identity. In some embodiments, any 2 of said six or more homologous distinct antigen components share at least about 30% sequence identity. In some embodiments, any 2 of said six or more homologous distinct antigen components share at least about 35% sequence identity. In some embodiments, said vaccine composition further comprises one or more non-homologous antigen components. In some embodiments, said vaccine composition further comprises an adjuvant. In some embodiments, said six or more homologous distinct antigen components are present in said vaccine composition at the same concentration/amount. In some embodiments, said six or more homologous distinct antigen components are present in said vaccine composition at the distinct concentrations/amounts. In some embodiments, a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components. In some embodiments, a dose of a homologous distinct antigen components of said six or more homologous distinct antigen components is calculated to be proportional to its average phylogenetic distance from the other homologous distinct antigen components of said six or more homologous distinct antigen components. In some embodiments, said vaccine composition comprises a fragment of a SARS virus. In some embodiments, said vaccine composition comprises a fragment of an influenza virus. In some embodiments, said vaccine composition comprises a fragment of an HIV virus. In some embodiments, said vaccine composition comprises a fragment of a SARS1 virus.

Another aspect of the present disclosure provides a vaccine composition comprising six or more homologous distinct antigen components, wherein two of said six or more homologous distinct antigen components share less than 95% sequence identity, and wherein the homologous distinct antigen components comprise proteins, and wherein a concentration/amount of a protein in a dose of said vaccine composition is about 1 nanogram (ng) to about 1 microgram (μg); wherein the homologous distinct antigen components comprise a plurality of RNA, and wherein a concentration/amount of an RNA of said plurality of RNA in a dose of said vaccine composition is about 1 ng to about 2.5 μg per dose; or wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said vaccine composition is about 1 ng to about 2.5 μg per dose, wherein the vaccine composition is a human pediatric vaccine composition. In some embodiments, said vaccine composition is for administration in a child between the ages of about 1 day old and about 18 years old. In some embodiments, said vaccine composition is for administration in a child between the ages of about 1 day old and about 5 years old. In some embodiments, said vaccine composition is for administration in a child between the ages of about 5 years old and about 18 years old. In some embodiments, a concentration/amount of a homologous distinct antigen components component of said homologous distinct antigen components in a dose of said vaccine composition is about 1 nanogram (ng) to about 0.5 micrograms (μg). In some embodiments, a concentration/amount of a homologous distinct antigen components component of said homologous distinct antigen components in a dose of said vaccine composition is about 1 nanogram (ng) to about 0.1 micrograms (μg). In some embodiments, the homologous distinct antigen components comprise a component of a virus. In some embodiments, the component of the virus is a receptor-binding domain. In some embodiments, at least two homologous distinct antigen components of said six or more homologous distinct antigen components comprise receptor binding domains for a cell surface protein. In some embodiments, said cell surface protein is mammalian. In some embodiments, said receptor binding domains of said at least two homologous distinct antigen components of said six or more homologous distinct antigen components share less than 99% sequence identity. In some embodiments, said receptor binding domains of said at least two homologous distinct antigen components of said six or more homologous distinct antigen components share less than 98% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 90% sequence identity. In some embodiments, said receptor binding domains of said at least two homologous distinct antigen components of said six or more homologous distinct antigen components share less than 85% sequence identity. In some embodiments, said receptor binding domains of said at least two homologous distinct antigen components of said six or more homologous distinct antigen components share less than 80% sequence identity. In some embodiments, said receptor binding domains of said at least two homologous distinct antigen components of said six or more homologous distinct antigen components share less than 75% sequence identity. In some embodiments, said receptor binding domains of said at least two homologous distinct antigen components of said six or more homologous distinct antigen components share less than 70% sequence identity. In some embodiments, an administration of said vaccine composition reduces a dominant population of immunogenic single variant epitopes corresponding to said homologous distinct antigen components. In some embodiments, any 2 of said six or more homologous distinct antigen components share less than about 90% sequence identity. In some embodiments, any 2 of said six or more homologous distinct antigen components share less than about 85% sequence identity. In some embodiments, any 2 of said six or more homologous distinct antigen components share less than about 80% sequence identity. In some embodiments, any 2 of said six or more homologous distinct antigen components share less than about 75% sequence identity. In some embodiments, any 2 of said six or more homologous distinct antigen components share less than about 70% sequence identity. In some embodiments, said vaccine composition comprises 7 or more homologous distinct antigen components. In some embodiments, said vaccine composition comprises 10 or more homologous distinct antigen components. In some embodiments, said vaccine composition comprises 15 or more homologous distinct antigen components. In some embodiments, said vaccine composition comprises 20 or more homologous distinct antigen components. In some embodiments, said vaccine composition comprises 25 or more homologous distinct antigen components. In some embodiments, said vaccine composition comprises 30 or more homologous distinct antigen components. In some embodiments, any 2 of said six or more homologous distinct antigen components share at least about 25% sequence identity. In some embodiments, any 2 of said six or more homologous distinct antigen components share at least about 30% sequence identity. In some embodiments, any 2 of said six or more homologous distinct antigen components share at least about 35% sequence identity. In some embodiments, said vaccine composition further comprises one or more non-homologous antigen components. In some embodiments, said vaccine composition further comprises an adjuvant. In some embodiments, said six or more homologous distinct antigen components are present in said vaccine composition at the same concentration. In some embodiments, said six or more homologous distinct antigen components are present in said vaccine composition at the distinct concentrations/amounts. In some embodiments, a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components. In some embodiments, a dose of a homologous distinct antigen components of said six or more homologous distinct antigen components is calculated to be proportional to its average phylogenetic distance from the other homologous distinct antigen components of said six or more homologous distinct antigen components. In some embodiments, said vaccine composition comprises a fragment of a SARS virus. In some embodiments, said vaccine composition comprises a fragment of an influenza virus. In some embodiments, said vaccine composition comprises a fragment of an HIV virus. In some embodiments, said vaccine composition comprises a fragment of a SARS1 virus.

Another aspect of the present disclosure provides a vaccine composition comprising six or more homologous distinct antigen components, wherein two of said six or more homologous distinct antigen components share less than 95% sequence identity, and wherein the homologous distinct antigen components comprise proteins, and wherein a concentration/amount of a protein in a dose of said vaccine composition is about 1 nanogram (ng) to about 3 microgram (μg); wherein the homologous distinct antigen components comprise a plurality of RNA, and wherein a concentration/amount of an RNA of said plurality of RNA in a dose of said vaccine composition is about 1 ng to about 5 μg per dose; or wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said vaccine composition is about 1 ng to about 5 μg per dose. wherein the vaccine composition is a veterinary vaccine composition. In some embodiments, said vaccine composition is for administration in a livestock animal. In some embodiments, said vaccine composition is for administration in a cow. In some embodiments, said vaccine composition is for administration in a sheep. In some embodiments, said vaccine composition is for administration in a pig. In some embodiments, said vaccine composition is for administration in a bird. In some embodiments, said vaccine composition is for administration in a canine animal. In some embodiments, said vaccine composition is for administration in a feline animal. In some embodiments, a concentration/amount of a homologous distinct antigen components component of said homologous distinct antigen components in a dose of said vaccine composition is about 1 nanogram (ng) to about 3 micrograms (μg). In some embodiments, a concentration/amount of a homologous distinct antigen components component of said homologous distinct antigen components in a dose of said vaccine composition is about 1 nanogram (ng) to about 1 microgram (μg). In some embodiments, a concentration/amount of a homologous distinct antigen components component of said homologous distinct antigen components in a dose of said vaccine composition is about 1 nanogram (ng) to about 0.1 microgram (μg). In some embodiments, the homologous distinct antigen components comprise a component of a virus. In some embodiments, the component of the virus is a receptor-binding domain. In some embodiments, at least two homologous distinct antigen components of said six or more homologous distinct antigen components comprise receptor binding domains for a cell surface protein. In some embodiments, said cell surface protein is mammalian. In some embodiments, said receptor binding domains of said at least two homologous distinct antigen components of said six or more homologous distinct antigen components share less than 99% sequence identity. In some embodiments, said receptor binding domains of said at least two homologous distinct antigen components of said six or more homologous distinct antigen components share less than 98% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 95% sequence identity. In some embodiments, said receptor binding domains of said at least two homologous distinct antigen components of said six or more homologous distinct antigen components share less than 90% sequence identity. In some embodiments, said receptor binding domains of said at least two homologous distinct antigen components of said six or more homologous distinct antigen components share less than 85% sequence identity. In some embodiments, said receptor binding domains of said at least two homologous distinct antigen components of said six or more homologous distinct antigen components share less than 80% sequence identity. In some embodiments, said receptor binding domains of said at least two homologous distinct antigen components of said six or more homologous distinct antigen components share less than 75% sequence identity. In some embodiments, said receptor binding domains of said at least two homologous distinct antigen components of said six or more homologous distinct antigen components share less than 70% sequence identity. In some embodiments, an administration of said vaccine composition reduces a dominant population of immunogenic single variant epitopes corresponding to said homologous distinct antigen components. In some embodiments, any 2 of said six or more homologous distinct antigen components share less than about 90% sequence identity. In some embodiments, any 2 of said six or more homologous distinct antigen components share less than about 85% sequence identity. In some embodiments, any 2 of said six or more homologous distinct antigen components share less than about 80% sequence identity. In some embodiments, any 2 of said six or more homologous distinct antigen components share less than about 75% sequence identity. In some embodiments, any 2 of said six or more homologous distinct antigen components share less than about 70% sequence identity. In some embodiments, said vaccine composition comprises 7 or more homologous distinct antigen components. In some embodiments, said vaccine composition comprises 10 or more homologous distinct antigen components. In some embodiments, said vaccine composition comprises 15 or more homologous distinct antigen components. In some embodiments, said vaccine composition comprises 20 or more homologous distinct antigen components. In some embodiments, said vaccine composition comprises 25 or more homologous distinct antigen components. In some embodiments, said vaccine composition comprises 30 or more homologous distinct antigen components. In some embodiments, any 2 of said six or more homologous distinct antigen components share at least about 25% sequence identity. In some embodiments, any 2 of said six or more homologous distinct antigen components share at least about 30% sequence identity. In some embodiments, any 2 of said six or more homologous distinct antigen components share at least about 35% sequence identity. In some embodiments, said vaccine composition further comprises one or more non-homologous antigen components. In some embodiments, said vaccine composition further comprises an adjuvant. In some embodiments, said six or more homologous distinct antigen components are present in said vaccine composition at the same concentration. In some embodiments, said six or more homologous distinct antigen components are present in said vaccine composition at the distinct concentrations/amounts. In some embodiments, a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components. In some embodiments, a dose of a homologous distinct antigen components of said six or more homologous distinct antigen components is calculated to be proportional to its average phylogenetic distance from the other homologous distinct antigen components of said six or more homologous distinct antigen components. In some embodiments, said vaccine composition comprises a fragment of a SARS virus. In some embodiments, said vaccine composition comprises a fragment of an influenza virus. In some embodiments, said vaccine composition comprises a fragment of an HIV virus. In some embodiments, said vaccine composition comprises a fragment of a SARS1 virus.

Another aspect of the present disclosure provides a method of inducing an immune response against a pathogen in a subject comprising administering the vaccine composition as disclosed herein. In some embodiments, the method is for prophylaxis against disease caused by the pathogen. Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts the number of FDA-approved vaccines for various doses per component of vaccine (μg). In no case is there an FDA-approved vaccine with a per-component dose lower than 3.75 ug.

FIG. 2A depicts the amount of induced antibodies measured by ELISA reactive to a particular recombinant influenza hemagglutinin antigen in the serum of mice in response to vaccination with either Centi-Flu (0.031 ug of mRNA/antigen), Flu-Biv (0.25 ug/antigen), or Flu-Biv (0.031 ug/antigen). Reactivity is shown to antigens present in Centi-Flu (H3N2 Hong Kong/1/1968, H3N2 Alaska/01/2021), present in Centi-Flu and Flu-Biv (California/07/2004), or heterologous antigens present in neither (H3N2 A/Victoria/361/2011, A/Maryland/02/2021). FIG. 2B depicts the amount of induced antibodies measured by ELISA reactive to a particular recombinant influenza hemagglutinin antigen in the serum of mice in response to vaccination with either Centi-Flu (0.25 ug of mRNA/antigen), Flu-Biv (2 ug/antigen), or Flu-Biv (0.25 ug/antigen). Reactivity is shown to antigens present in Centi-Flu (H3N2 Hong Kong/1/1968, H3N2 Alaska/01/2021), present in Centi-Flu and Flu-Biv (California/07/2004), or heterologous antigens present in neither (H3N2 A/Victoria/361/2011, A/Maryland/02/2021).

FIG. 3A depicts serum reactivity measured by ELISA in ferrets immunized with 1 μg of mRNA encoding influenza hemagglutinin antigens of H3N2 Alaska/01/2021, H3N2 California/07/2004, H3N2 Cambodia/2020, H3N2 Indiana/11/2018, H3N2 Bilthoven/1761/1976, H3N2 Indiana/08/2011, H3N2 Nanchang/933/1995, or H3N2 Memphis/i/1980, to the same antigen that the animal was immunized with; FIG. 3B depicts serum reactivity measured by ELISA in ferrets immunized with approximately 0.5 ug of mRNA per antigen encoding 8 influenza hemagglutinin antigens of H3N2 Alaska/01/2021, H3N2 California/07/2004, H3N2 Cambodia/2020, H3N2 Indiana/11/2018, H3N2 Bilthoven/1761/1976, H3N2 Nanchang/933/1995, H3N2 Memphis/1/1980, H3N2 Hong Kong/1/1968, to the indicated recombinant protein antigens.

FIG. 4 depicts serum reactivity measured by ELISA in mice immunized with either Centi-HIV (10 recombinant gp160 antigens; human-equivalent dose (HED) of 1 ug/antigen=total 10 ug), or a single recombinant HIV gp160 antigen (HED 1 ug or 10 ug).

FIG. 5 depicts serum reactivity measured by ELISA to snake venom from Crotalus horridus, in mice immunized with either a mixture of 15 snake venoms (effective HED of 3 ug/ag=45 ug total), or Crotalus horridus snake venom alone (effective HED of 3 μg or 45 ug).

FIG. 6 depicts pairwise sequence identity between a set of H3N2 hemagglutinin homologous distinct antigen components.

FIG. 7 depicts pairwise sequence identity between a set of HIV gp160 homologous distinct antigen components.

FIG. 8 depicts pairwise sequence identity between a set of snake venom PLA2 homologous distinct antigen components.

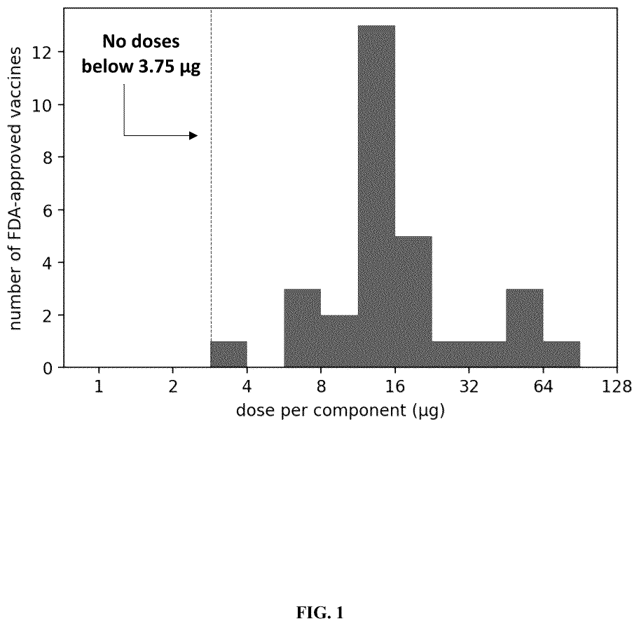

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Vaccines may comprise multiple homologous antigen components, especially for pathogens that belong to diverse phylogenetic families (e.g., influenza). For any particular antigen component, there may be a minimum dose of that antigen component sufficient to elicit an optimal immune response against that antigen alone. This present disclosure describes vaccine compositions comprising a mixture of multiple homologous antigen components, wherein each antigen component is dosed below such a threshold.

In some embodiments, the vaccine composition is an RNA vaccine. For RNA vaccines, a typical dose per antigen component may be at least about 30 μg to at least about 100 ug. Examples of such RNA vaccines may include Pfizer or Modema COVID vaccines. Dose escalation studies indicate that about 10 μg may be a threshold dose for adults that is insufficient to elicit a sufficient immune response. The RNA may be a messenger RNA (mRNA). The mRNA may encode an antigen protein. The antigen may be an influenza protein or fragment thereof. The antigen may be a hemagglutinin protein or fragment thereof. In the context of RNA vaccines as disclosed herein, an “antigen component” can refer to an mRNA encoding a protein component towards which the vaccine is directed. In the context of RNA vaccines as disclosed herein, sequence identity between two antigen components can refer to the sequence identity between the amino acid sequences of the protein components for which the mRNA encodes.

In the context of non-RNA vaccines as disclosed herein, “antigen component” can refer to a protein component towards which the antigen is directed.

In some embodiments, the vaccine composition is a virus like particle (VLP) vaccine. For VLP vaccines, a typical dose per antigen component is at least about 20 μg to at least about 40 ug. A minimum reported dose may be about 5 ug, for infants, children and adolescents. A minimum reported dose may be about 10 ug, for adults. Example of vaccines with a minimum dose of about 5 μg for infants, children, and adolescents include RECOMBIVAX HB. Examples of vaccines with a minimum dose of about 10 ug for adults include RECOMBIVAX HB.

The minimum per-antigen dose of any clinically approved vaccine in a particular format implies that dosing below that dose either does not elicit a protective immune response, or that the elicited immune response is sub-optimal compared to a higher dose. The present disclosure provides a novel approach for vaccines where 5 or more antigens are combined into a single vaccine, where each individual antigen dose is below a minimum dose utilized in any clinically approved vaccine of that format. This may allow the selective stimulation of B cells that recognize broadly conserved epitopes by coupling the conservation of these epitopes to their concentration/amount in the vaccine formulation. By administering a mixture of many (i.e., ≥5) diverse variants of recombinant antigen, each individually below the threshold dose necessary to elicit an optimal strain-specific immune response, vaccine compositions as disclosed herein may reduce the dominant population of immunogenic single variant epitopes, leaving conserved epitopes (found across multiple components) at a higher effective dose. Conserved epitopes may be primarily presented on vaccine compositions as claimed herein, despite each immunogen retaining its full, native structure. Furthermore, conserved conformational epitopes are fully preserved.

In some embodiments, two antigen components are considered ‘distinct’ if their amino acid sequences, or the amino acid sequences they correspond to (in the case of RNA vaccines), share less than about 99%, 98%, 97%, 96%, 95%, 90%, 85%, 80%, 75%, or 70% sequence identity.

In other embodiments, two antigen components are considered ‘distinct’ if they comprise receptor binding domains for a mammalian cell surface protein, and the amino acid sequences of their respective receptor binding domains share less than about 99%, 98%, 96%, 95%, 90%, 85%, 80%, 75%, or 70% sequence identity.

In some embodiments, a vaccine composition of this invention comprises six or more homologous distinct antigen components. In some embodiments, any 2 of said six or more homologous distinct antigen components share at least about 30% sequence identity. In some embodiments, any 2 of said six or more homologous distinct antigen components share at least about 40% sequence identity. In some embodiments, any 2 of said six or more homologous distinct antigen components share at least about 50% sequence identity. In some embodiments, any 2 of said six or more homologous distinct antigen components share at least about 60% sequence identity. In some embodiments, any 2 of said six or more homologous distinct antigen components share at least about 70% sequence identity. In some embodiments, any 2 of said six or more homologous distinct antigen components share at least about 80% sequence identity.

In some embodiments, a vaccine composition of this invention comprises six or more RNAs encoding homologous distinct antigen components. In some embodiments, each RNA is present in an amount of at least 10 nanograms per dose. In some embodiments, each RNA is present in an amount of at least 100 nanograms per dose. In some embodiments, each RNA is present in an amount of at least 250 nanograms per dose. In some embodiments, each RNA is present in an amount of at least 500 nanograms per dose.

The vaccine compositions of this invention comprise five or more homologous antigen components, wherein each pair is distinct (“the distinct set”). The distinct set may comprise about 5, 6, 7, 8, 9, 10, 11, 12, 13, 14, 15, 16, 17, 18, 19, 20, 21, 22, 23, 24, 25, 26, 27, 28, 29, 30, or more than 30 homologous antigen components. The vaccine composition may further comprise additional homologous antigen components that are not distinct relative to the distinct set, or distinct relative to each other. For example, a vaccine composition of this invention may comprise 8 antigen components; wherein components 1-6 form a distinct set in which any 2 share less than 95% sequence identity, and components 7-8 share greater than 95% sequence identity to each other. As another example, a vaccine composition of this invention may comprise 8 antigen components; wherein components 1-6 form a distinct set in which any 2 share less than 95% sequence identity, and components 7-8 share greater than 95% sequence identity to component 1. The vaccine composition may further comprise additional non-homologous antigen components.

In some embodiments, the dose of each antigen component in a protein vaccine intended for adult use is greater than about 1 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in a protein vaccine intended for adult use is greater than about 600 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, or less than about 1 μg per dose.

In some embodiments, the dose of each antigen component in a protein vaccine intended for pediatric use is greater than about 1 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in a protein vaccine intended for pediatric use is greater than about 600 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, or less than about 1 μg per dose.

In some embodiments, the dose of each antigen component in a protein vaccine intended for veterinary use is greater than about 1 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose.

In some embodiments, the dose of each antigen component in an RNA vaccine intended for adult use is greater than about 1 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in an RNA vaccine intended for adult use is greater than about 10 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in an RNA vaccine intended for adult use is greater than about 100 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, or less than about 0.5 μg per dose.

In some embodiments, the dose of each antigen component in an RNA vaccine intended for pediatric use is greater than about 1 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in an RNA vaccine intended for pediatric use is greater than about 10 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in an RNA vaccine intended for pediatric use is greater than about 100 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, or less than about 0.5 μg per dose.

In some embodiments, the dose of each antigen component in an RNA vaccine intended for veterinary use is greater than about 1 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in an RNA vaccine intended for veterinary use is greater than about 10 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, less than about 0.5 μg per dose, or less than about 0.1 μg per dose. In some embodiments, the dose of each antigen component in an RNA vaccine intended for veterinary use is greater than about 100 ng per dose, but less than about 10 μg per dose, less than about 9 μg per dose, less than about 8 μg per dose, less than about 7 μg per dose, less than about 6 μg per dose, less than about 5 μg per dose, less than about 4 μg per dose, less than about 3 μg per dose, less than about 2 μg per dose, less than about 1 μg per dose, or less than about 0.5 μg per dose.

In some embodiments, a vaccine composition of this invention comprises RNA encoding for 6 or more homologous distinct antigen components; wherein each pair of antigens shares between 25% and 96% sequence identity, and the amount of each RNA is between 50 ng per dose and 2 μg per dose. In some embodiments, a human adult vaccine composition of this invention comprises RNA encoding for 6 or more homologous distinct antigen components; wherein each pair of antigens shares between 25% and 96% sequence identity, and the amount of each RNA is between 50 ng per dose and 5 μg per dose. In some embodiments, a human pediatric vaccine composition of this invention comprises RNA encoding for 6 or more homologous distinct antigen components; wherein each pair of antigens shares between 25% and 96% sequence identity, and the amount of each RNA is between 10 ng per dose and 2 μg per dose.

In some embodiments, a vaccine composition of this invention comprises 6 or more homologous distinct antigen components; wherein each pair of antigens shares between 25% and 96% sequence identity, and the amount of each antigen component is between 600 ng per dose and 3 μg per dose.

In some embodiments, the antigen components may be inactivated viruses. In this case, the dose of a given protein of antigenic interest is calculated as:

dose of a given protein of antigenic interest=(estimated number of viral particles in the dose)×(copy number of the protein of antigenic interest per viral particle)×(molecular weight of the protein of antigenic interest).

In some embodiments, the vaccine composition may further comprise an adjuvant.

In some embodiments, each antigen component in the vaccine composition may be present at the same dose as the other antigen components.

In other embodiments, each antigen component in the vaccine composition may be present at a different dose from the other antigen components. The dose of a given antigenic component may be calculated to be proportional to the phylogenetic distance of the component to its nearest other component; or calculated to be proportional to its average phylogenetic distance to all other components.

For protein subunit vaccines, the typical dose per antigen component may be at least about 15 μg to at least about 30 μg for adults. For protein subunit vaccines, the typical dose per antigen component may be at least about 7.5 μg to at least about 15 μg for pediatrics. The lowest dose for a clinically approved protein subunit vaccine may be at least about 3.75 μg. Examples of such clinically approved protein subunit vaccines may include monovalent H5N1 influenza HA vaccine (ID Biomedical Corporation).

In some embodiments, the invention comprises a human adult vaccine composition comprising 6 or more homologous distinct antigen components, wherein each pair of antigens shares less than about 95% sequence identity, and the antigen components are proteins, and each distinct antigen component is less than about 3 μg per dose; or the antigen components are RNA, and each distinct antigen component is less than about 5 μg per dose; or the antigen components are proteins displayed on heterologous VLPs, and each distinct antigen component is less than about 5 μg per dose. In some embodiments, the invention comprises a human adult vaccine composition comprising 6 or more homologous distinct antigen components, wherein each pair of antigens shares less than about 95% sequence identity, and the antigen components are proteins, and each distinct antigen component is less than about 3 μg per dose and greater than about 600 ng per dose. In some embodiments, the invention comprises a human adult vaccine composition comprising mRNA encoding for 6 or more homologous distinct antigen components, wherein each pair of antigen components shares less than about 95% sequence identity, and mRNA encoding for each distinct antigen component is less than about 5 μg per dose and greater than about 50 ng per dose.

In some embodiments, the invention comprises a human pediatric vaccine composition comprising 6 or more homologous distinct antigen components, wherein each pair of antigens shares less than about 95% sequence identity, and the antigen components are proteins, and each distinct antigen component is less than about 1 μg per dose; or the antigen components are RNA, and each distinct antigen component is less than about 2.5 μg per dose; or the antigen components are proteins displayed on heterologous VLPs, and each distinct antigen component is less than about 2.5 μg per dose. In some embodiments, the invention comprises a human pediatric vaccine composition comprising mRNA encoding for 6 or more homologous distinct antigen components, wherein each pair of antigen components shares less than about 95% sequence identity, and mRNA encoding for each distinct antigen component is less than about 1 μg per dose and greater than about 10 ng per dose.

In some embodiments, the invention comprises a veterinary vaccine composition comprising 6 or more homologous distinct antigen components, wherein each pair of antigens shares less than about 95% sequence identity, and the antigen components are proteins, and each distinct antigen component is less than about 3 μg per dose; or the antigen components are RNA, and each distinct antigen component is less than about 5 μg per dose; or the antigen components are proteins displayed on heterologous VLPs, and each distinct antigen component is less than about 5 μg per dose.

In some embodiments, the vaccine compositions of the invention comprise RNAs encoding homologous distinct antigen components. In some embodiments, the RNAs are messenger RNA (mRNA). In some embodiments, the RNAs are encapsulated in lipid nanoparticles (LNPs). In some embodiments, the LNP comprises an ionizable or cationic lipid, a helper phospholipid, a PEG-conjugated lipid, and a cholesterol-based lipid. In some embodiments, the ionizable lipid is ALC-0315, SM-102, or MC-3. In some embodiments, the cholesterol-based lipid is cholesterol. In some embodiments, the helper phospholipid is DSPC. In some embodiments, the PEG-conjugated lipid is ALC-0159. In some embodiments, the ionizable lipid is present at a molar ratio of 35-55%. In some embodiments, the PEG-conjugated lipid is present at a molar ratio of 0.25% to 3%. In some embodiments, the helper phospholipid is present at a molar ratio of 5% to 20%. In some embodiments, the cholesterol-based lipid is present at a molar ratio of 20% to 45%. All lipid molar ratios described here are relative to the total lipid content of the LNP.

In certain embodiments, the mRNA comprises at least one chemical modification. In certain embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified. In certain embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified. In certain embodiments, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In certain embodiments, the chemical modification is selected from the group consisting of pseudouridine, N1-methylpseudouridine, 5-methylcytosine, 5-methoxyuridine, and a combination thereof. In certain embodiments, the chemical modification is N1-methylpseudouridine.

### Influenza Antigens

The present disclosure provides vaccines comprising a plurality of RNAs encoding six or more homologous distinct antigen components. The vaccines may be for the prevention of influenza. The six or more homologous distinct antigen components may comprise one or more influenza proteins or one or more fragments thereof. The six or more homologous distinct antigen components may comprise hemagglutinin components. In some embodiments, one or more hemagglutinin components are from an H1N1 subtype. In some embodiments, one or more hemagglutinin components are from an H3N2 subtype.

In some embodiments, the invention comprises a human adult vaccine composition comprising a plurality of RNAs encoding six or more homologous distinct antigen components, wherein the homologous distinct antigen components are influenza hemagglutinins or fragments thereof, wherein each pair of antigen components shares less than about 96% sequence identity, and the amount of each mRNA is less than about 5 μg per dose. In some embodiments, the invention comprises a vaccine composition comprising a plurality of RNAs encoding six or more homologous distinct antigen components, wherein the homologous distinct antigen components are influenza hemagglutinins or fragments thereof, wherein each pair of antigen components shares less than about 96% sequence identity, and the amount of each mRNA is less than about 4 μg per dose, less than about 3 μg per dose, less than about 1 μg per dose, less than about 0.5 μg per dose, or less than about 0.1 μg per dose. In some embodiments, the invention comprises a vaccine composition comprising a plurality of RNAs encoding six or more homologous distinct antigen components, wherein the homologous distinct antigen components are influenza hemagglutinins or fragments thereof, wherein each pair of antigen components shares less than about 95% sequence identity, and the amount of each mRNA is less than about 4 μg per dose, less than about 3 μg per dose, less than about 1 μg per dose, less than about 0.5 μg per dose, or less than about 0.1 μg per dose.

In some embodiments, a vaccine composition of this invention comprises RNA encoding for 6 or more homologous distinct antigen components; wherein the homologous distinct antigen components are influenza hemagglutinins or fragments thereof. In some embodiments, each pair of said antigens shares between 25% and 96% sequence identity, and the amount of each RNA is between 50 ng per dose and 2 μg per dose. In some embodiments, the amount of each RNA is between 50 ng per dose and 5 μg per dose. In some embodiments intended for pediatric use, the amount of each RNA is between 10 ng per dose and 2 μg per dose.

The present disclosure provides vaccines for the prevention of influenza. In some embodiments, one or more hemagglutinin components are from one of more strains of influenza. In some embodiments, the one or more strains of influenza comprises A/Wisconsin/588/2019. In some embodiments, the one or more strains of influenza comprises A/California/07/2009. In some embodiments, the one or more strains of influenza comprises A/Denver/57. In some embodiments, the one or more strains of influenza comprises A/Brisbane/59/2007. In some embodiments, the one or more strains of influenza comprises A/Beijing/262/1995. In some embodiments, the one or more strains of influenza comprises A/Puerto_Rico/8/1934. In some embodiments, the one or more strains of influenza comprises A/New_York/1/1918. In some embodiments, the one or more strains of influenza comprises A/Wisconsin/28/2011.2011/12. In some embodiments, the one or more strains of influenza comprises A/Indiana/11/2018. In some embodiments, the one or more strains of influenza comprises A/California/07/2004. In some embodiments, the one or more strains of influenza comprises A/Alaska/01/2021.

In some embodiments, the one or more strains of influenza comprises A/Cambodia/e0826360/2020. In some embodiments, the one or more strains of influenza comprises A/Nanchang/933/1995. In some embodiments, the one or more strains of influenza comprises A/Memphis/1/1980. In some embodiments, the one or more strains of influenza comprises A/Bilthoven/1761/1976. In some embodiments, the one or more strains of influenza comprises A/Hong_Kong/1/1968. In some embodiments, the one or more strains of influenza comprises A/Indiana/08/2011. In some embodiments, an antigen included in or encoded by the vaccine has at least about 70%, at least about 75%, at least about 80%, at least about 85%, at least about 90%, at least about 95%, or about 100% sequence identity to the amino acid sequence of any one of SEQ ID NOS: 3, 5, 7, 9, 11, 13, 15, 17, 19, 21, 23, 25, 27, 29, 31, 33, or 35.

The present disclosure provides vaccines for the prevention of influenza, wherein the vaccines comprise a plurality of RNA encoding a plurality of distinct homologous antigen components. The distinct homologous antigen components may be influenza proteins or fragments thereof. In some embodiments, an RNA of said plurality of RNAs is a messenger RNA (mRNA). In some embodiments, the mRNA encodes an influenza antigen or fragment thereof. In some embodiments, the mRNA encodes influenza hemagglutinin or a fragment thereof. In some embodiments, the mRNA encoding the influenza antigen comprises an mRNA insert region. In some embodiments, the mRNA insert region has at least about 70%, at least about 75%, at least about 80%, at least about 85%, at least about 90%, at least about 95%, or about 100% identity to any one of SEQ ID NOS: 4, 6, 8, 10, 12, 14, 16, 18, 20, 22, 24, 26, 28, 30, 32, 34, or 36, wherein the thymines in any one of SEQ ID NOS: 4, 6, 8, 10, 12, 14, 16, 18, 20, 22, 24, 26, 28, 30, 32, 34, or 36 are replaced with uridines, uridine isomers, pseudouridines, pseudouridine methylated derivatives, or N1-methyl-pseudouridines. In some embodiments, the mRNA encoding the influenza antigen comprises a 3′ UTR region. In some embodiments, the 3′ UTR region has at least about 70%, at least about 75%, at least about 80%, at least about 85%, at least about 90%, at least about 95%, or about 100% identity to SEQ ID NO: 2, wherein the thymines in SEQ ID NO: 2 are replaced with uridines, uridine isomers, pseudouridines, pseudouridine methylated derivatives, or N1-methyl-pseudouridines. In some embodiments, the mRNA encoding the influenza antigen comprises a 5′ UTR region. In some embodiments, the 5′ UTR comprises at least about 70%, at least about 75%, at least about 80%, at least about 85%, at least about 90%, at least about 95%, or about 100% identity to SEQ ID NO: 1, wherein the thymines in SEQ ID NO: 1 are replaced with uridines, uridine isomers, pseudouridines, pseudouridine methylated derivatives, or N1-methyl-pseudouridines.

TABLE 1
Table of Sequences for Vaccines

| SEQ ID NO: | Sequence | Description |
|---|---|---|
| 1 | GAGAATAAACTAGTATTCTTCTGGTCCCCACA | 5′ UTR sequence |
| | GACTCAGAGAGAACCCGCCACCATGTTCGTGT | |
| | TCCTGGTGCTGCTGCCTCTGGTGTCCAGCCAGT | |
| | GTGTG | |
| 2 | TGATGACTCGAGCTGGTACTGCATGCACGCAA | 3′ UTR sequence |
| | TGCTAGCTGCCCCTTTCCCGTCCTGGGTACCCC | |
| | GAGTCTCCCCCGACCTCGGGTCCCAGGTATGC | |
| | TCCCACCTCCACCTGCCCCACTCACCACCTCTG | |
| | CTAGTTCCAGACACCTCCCAAGCACGCAGCAA | |
| | TGCAGCTCAAAACGCTTAGCCTAGCCACACCC | |
| | CCACGGGAAACAGCAGTGATTAACCTTTAGCA | |
| | ATAAACGAAAGTTTAACTAAGCTATACTAACC | |
| | CCAGGGTTGGTCAATTTCGTGCCAGCCACACC | |
| | CTGGAGCTAGCA | |
| 3 | ADTLCIGYHANNSTDTVDTVLEKNVTVTHSVNL | A/Wisconsin/588/2019 |
| | LEDKHNGKLCKLRGVAPLHLGKCNIAGWILGNP | mature HA Amino |
| | ECESLSTARSWSYIVETSNSDNGTCYPGDFINYEE | Acid Sequence |
| | LREQLSSVSSFERFEIFPKTSSWPNHDSDNGVTAA | |
| | CPHAGAKSFYKNLIWLVKKGKSYPKINQTYIND | |
| | KGKEVLVLWGIHHPPTIADQQSLYQNADAYVFV | |
| | GTSRYSKKFKPEIATRPKVRDQEGRMNYYWTLV | |
| | EPGDKITFEATGNLVAPRYAFTMERDAGSGIIISD | |
| | TPVHDCNTTCQTPEGAINTSLPFQNVHPITIGKCP | |
| | KYVKSTKLRLATGLRNVPSIQSRGLFGAIAGFIEG | |
| | GWTGMVDGWYGYHHQNEQGSGYAADLKSTQN | |
| | AIDKITNKVNSVIEKMNTQFTAVGKEFNHLEKRI | |
| | ENLNKKVDDGFLDIWTYNAELLVLLENERTLDY | |
| | HDSNVKNLYEKVRNQLKNNAKEIGNGCFEFYHK | |
| | CDNTCMESVKNGTYDYPKYSEEAKLNREKIDGV | |
| | KLDS | |
| 4 | GCTGACACCCTGTGCATTGGCTATCATGCCAA | A/Wisconsin/588/2019 |
| | CAACTCCACAGACACCGTGGACACAGTGCTGG | mature HA nucleic acid |
| | AGAAGAACGTGACAGTGACCCACTCCGTGAAC | sequence |
| | CTGCTGGAAGACAAGCACAACGGCAAACTGTG | |
| | CAAGCTGAGAGGCGTGGCCCCTCTGCACCTGG | |
| | GCAAGTGCAACATCGCCGGCTGGATCCTGGGC | |
| | AACCCAGAGTGCGAGAGCCTGTCCACAGCCCG | |
| | GTCCTGGTCTTACATTGTGGAGACATCCAACA | |
| | GCGACAACGGCACCTGCTACCCTGGCGACTTC | |
| | ATCAATTACGAGGAGCTGAGAGAGCAGCTGAG | |
| | CTCCGTGTCCAGCTTCGAGAGGTTCGAGATCTT | |
| | TCCCAAGACAAGCTCCTGGCCCAACCACGACA | |
| | GCGACAACGGGGTGACAGCCGCCTGCCCTCAC | |
| | GCAGGCGCCAAGAGCTTCTACAAAAACCTGAT | |
| | CTGGCTCGTGAAGAAAGGAAAGAGCTACCCCA | |
| | AGATCAACCAGACCTACATCAACGACAAGGGC | |
| | AAGGAGGTGCTGGTGCTTTGGGGCATCCACCA | |
| | CCCTCCCACAATTGCCGACCAGCAGTCCCTGT | |
| | ACCAGAACGCTGACGCCTATGTCTTCGTGGGC | |
| | ACCTCTAGATACAGCAAGAAGTTCAAGCCTGA | |
| | GATCGCCACAAGACCCAAGGTGCGGGATCAGG | |
| | AGGGCCGGATGAATTACTACTGGACCCTGGTG | |
| | GAGCCAGGCGACAAGATCACCTTCGAGGCCAC | |
| | CGGCAACCTGGTGGCTCCCCGATACGCCTTCA | |
| | CCATGGAGCGGGACGCCGGCAGCGGCATCATC | |
| | ATCAGCGACACACCTGTGCACGACTGCAACAC | |
| | CACCTGCCAGACCCCTGAAGGAGCCATCAACA | |
| | CCAGCCTGCCCTTCCAGAATGTCCACCCCATCA | |
| | CCATCGGCAAATGTCCAAAGTACGTGAAGTCC | |
| | ACCAAACTGAGACTGGCCACCGGCCTGAGAAA | |
| | CGTCCCCTCAATCCAGAGCAGAGGCCTGTTTG | |
| | GGGCCATTGCCGGCTTCATCGAGGGCGGATGG | |
| | ACCGGCATGGTCGACGGATGGTACGGATATCA | |
| | CCACCAGAACGAACAGGGAAGCGGCTACGCC | |
| | GCCGACCTGAAGTCCACACAGAACGCCATCGA | |
| | CAAGATCACCAACAAGGTGAACAGCGTGATCG | |
| | AGAAAATGAACACACAGTTCACAGCCGTGGGC | |
| | AAGGAATTCAATCACCTGGAGAAGAGGATCGA | |
| | GAATCTGAACAAGAAGGTGGACGACGGCTTCC | |
| | TGGACATCTGGACATACAACGCAGAGCTCCTG | |
| | GTGCTGCTGGAAAATGAGCGGACACTGGACTA | |
| | CCACGACTCCAACGTGAAGAACCTGTACGAGA | |
| | AGGTGCGGAATCAACTGAAGAACAACGCCAA | |
| | GGAGATTGGCAACGGCTGCTTTGAGTTCTACC | |
| | ACAAGTGCGACAACACCTGTATGGAGTCCGTG | |
| | AAGAACGGCACATATGACTACCCCAAGTACAG | |
| | CGAAGAAGCCAAGCTGAACAGAGAGAAGATT | |
| | GATGGAGTGAAACTGGACAGC | |
| 5 | ADTLCIGYHANNSTDTVDTVLEKNVTVTHSVNL | A/California/07/2009 |
| | LEDKHNGKLCKLRGVAPLHLGKCNIAGWILGNP | mature HA Amino |
| | ECESLSTASSWSYIVETPSSDNGTCYPGDFIDYEE | Acid Sequence |
| | LREQLSSVSSFERFEIFPKTSSWPNHDSNKGVTAA | |
| | CPHAGAKSFYKNLIWLVKKGNSYPKLSKSYIND | |
| | KGKEVLVLWGIHHPSTSADQQSLYQNADAYVFV | |
| | GSSRYSKKFKPEIAIRPKVRDQEGRMNYYWTLV | |
| | EPGDKITFEATGNLVVPRYAFAMERNAGSGIIISD | |
| | TPVHDCNTTCQTPKGAINTSLPFQNIHPITIGKCP | |
| | KYVKSTKLRLATGLRNIPSIQSRGLFGAIAGFIEG | |
| | GWTGMVDGWYGYHHQNEQGSGYAADLKSTQN | |
| | AIDEITNKVNSVIEKMNTQFTAVGKEFNHLEKRI | |
| | ENLNKKVDDGFLDIWTYNAELLVLLENERTLDY | |
| | HDSNVKNLYEKVRSQLKNNAKEIGNGCFEFYHK | |
| | CDNTCMESVKNGTYDYPKYSEEAKLNREEIDGV | |
| | KLES | |
| 6 | GCTGACACCCTGTGCATTGGCTATCATGCCAA | A/California/07/2009 |
| | CAACAGCACCGACACAGTGGACACAGTGCTGG | mature HA nucleic acid |
| | AAAAGAACGTGACCGTGACACACTCCGTGAAC | sequence |
| | CTGCTGGAGGACAAGCACAACGGCAAACTGTG | |
| | CAAGCTGAGAGGCGTGGCCCCTCTGCACCTGG | |
| | GCAAGTGCAACATCGCCGGCTGGATCCTGGGC | |
| | AACCCAGAGTGCGAGAGCCTGAGCACCGCCAG | |
| | CTCCTGGTCTTACATCGTGGAGACACCCAGCTC | |
| | CGACAACGGCACATGCTACCCTGGCGACTTCA | |
| | TCGACTACGAGGAACTGAGAGAGCAGCTGAGC | |
| | TCCGTGTCCTCTTTCGAGAGGTTCGAGATCTTC | |
| | CCCAAGACCTCATCTTGGCCCAACCACGACAG | |
| | CAACAAGGGCGTGACCGCCGCCTGTCCTCACG | |
| | CTGGCGCAAAGAGCTTCTACAAGAACCTGATC | |
| | TGGCTCGTGAAGAAGGGAAACAGCTACCCCAA | |
| | GCTGAGCAAGAGCTACATCAATGACAAAGGCA | |
| | AGGAGGTGCTGGTGCTATGGGGCATCCATCAC | |
| | CCCAGCACAAGCGCTGACCAGCAGAGCCTGTA | |
| | TCAGAACGCTGACGCCTACGTCTTCGTCGGCA | |
| | GCTCCCGGTACAGCAAGAAATTCAAGCCCGAG | |
| | ATCGCCATCCGGCCCAAGGTGCGGGACCAAGA | |
| | GGGCAGAATGAACTACTACTGGACCCTGGTGG | |
| | AGCCTGGCGACAAGATCACCTTTGAGGCCACA | |
| | GGCAATCTGGTGGTGCCCAGATACGCCTTTGC | |
| | CATGGAGAGAAATGCCGGCTCCGGCATCATCA | |
| | TCAGCGACACACCTGTGCACGACTGCAACACC | |
| | ACCTGTCAGACACCCAAGGGCGCCATCAACAC | |
| | AAGCCTGCCTTTTCAGAACATTCACCCCATCAC | |
| | CATTGGCAAGTGCCCCAAGTACGTCAAGAGCA | |
| | CCAAACTGCGGCTGGCCACAGGACTGCGGAAC | |
| | ATCCCCAGCATTCAGAGCCGGGGCCTGTTTGG | |
| | AGCCATCGCTGGCTTCATCGAGGGCGGCTGGA | |
| | CCGGCATGGTGGACGGCTGGTACGGCTACCAT | |
| | CACCAGAATGAACAGGGCAGCGGCTACGCCGC | |
| | TGACCTGAAGTCCACCCAGAACGCCATCGACG | |
| | AAATCACCAACAAGGTCAACTCTGTGATCGAA | |
| | AAGATGAACACCCAGTTCACCGCCGTGGGAAA | |
| | AGAGTTCAACCACCTGGAGAAGAGGATCGAA | |
| | AATCTGAACAAAAAGGTGGACGACGGCTTTCT | |
| | GGACATCTGGACCTACAACGCAGAGCTCCTGG | |
| | TGCTGCTGGAAAATGAAAGAACACTGGACTAC | |
| | CACGACTCCAACGTCAAGAACCTGTACGAGAA | |
| | GGTGCGGAGCCAGCTGAAGAACAACGCCAAG | |
| | GAGATCGGCAACGGCTGCTTCGAGTTCTACCA | |
| | CAAGTGCGACAACACCTGTATGGAGTCCGTGA | |
| | AGAACGGCACATACGACTACCCCAAGTACTCT | |
| | GAAGAGGCCAAGCTGAACAGAGAGGAGATTG | |
| | ATGGAGTGAAGCTGGAGTCC | |
| 7 | ADTICIGYHANNSTDTVDTVLEKNVTVTHSVNLL | A/Denver/57mature |
| | EDSHNGKLCRLKGKAPLQLGNCNIAGWVLGNPE | HA Amino Acid |
| | CESLLSNRSWSYIAETPNSENGTCYPGDFADYEE | Sequence |
| | LREQLSSVSSFERFEIFPKERSWPNHTTRGVTAAC | |
| | PHARKSSFYKNLVWLTEANGSYPNLSRSYVNNQ | |
| | EKEVLVLWGVHHPSNIEEQRALYRKDNAYVSVV | |
| | SSNYNRRFTPEIAKRPKVRDQSGRMNYYWTLLE | |
| | PGDTIIFEATGNLIAPWYAFALSRGPGSGIITSNAP | |
| | LDECDTKCQTPQGAINSSLPFQNIHPVTIGECPKY | |
| | VRSTKLRMVTGLRNIPSVQSRGLFGAIAGFIEGG | |
| | WTGMMDGWYGYHHQNEQGSGYAADQKSTQN | |
| | AINGITNKVNSVIEKMNTQFTAVGKEFNKLEKR | |
| | MENLNKKVDDGFMDIWTYNAELLVLLENERTL | |
| | DFHDSNVKNLYEKVKNQLRNNAKELGNGCFEF | |
| | YHKCDNECMESVKNGTYDYPKYSEESKLNREKI | |
| | DGVKLES | |
| 8 | GCCGACACCATCTGCATTGGATACCATGCCAA | HA nucleic acid |
| | CAACAGCACCGACACCGTGGACACAGTGCTGG | sequence |
| | AAAAGAACGTGACCGTGACCCACAGCGTGAAC | A/Denver/57mature |
| | CTGCTGGAAGACAGCCACAACGGCAAACTGTG | |
| | CAGACTGAAGGGCAAGGCCCCCCTGCAGCTGG | |
| | GAAACTGCAACATCGCCGGATGGGTGCTGGGA | |
| | AATCCCGAGTGCGAGAGCCTGCTGAGCAACAG | |
| | GAGCTGGAGCTACATCGCTGAAACCCCCAACA | |
| | GCGAGAACGGCACCTGCTATCCTGGCGACTTC | |
| | GCTGACTACGAGGAACTGCGGGAGCAGCTGTC | |
| | CTCCGTGTCCTCCTTTGAAAGATTTGAGATCTT | |
| | CCCCAAGGAGCGGAGCTGGCCCAACCACACAA | |
| | CCAGGGGCGTGACCGCCGCCTGCCCTCATGCC | |
| | AGAAAGAGCAGCTTCTACAAGAATCTGGTCTG | |
| | GCTGACCGAGGCCAATGGCAGCTATCCCAACC | |
| | TGTCCAGAAGCTACGTGAACAACCAGGAGAAG | |
| | GAGGTGCTGGTCCTGTGGGGGGTGCACCACCC | |
| | CAGCAACATCGAGGAGCAGAGAGCCCTGTACA | |
| | GAAAGGACAACGCCTATGTCAGCGTGGTGTCC | |
| | AGCAACTACAACAGACGGTTCACCCCCGAAAT | |
| | TGCCAAGCGGCCCAAGGTGCGGGATCAGAGCG | |
| | GCCGGATGAACTACTACTGGACCCTGCTGGAG | |
| | CAACCTGATCGCCCCTTGGTACGCCTTCGCCCT | |
| | GAGCAGAGGCCCTGGCAGCGGCATCATCACCA | |
| | GCAATGCCCCCCTGGACGAGTGCGACACAAAG | |
| | CCTGGAGACACCATCATCTTCGAGGCCACCGG | |
| | TGTCAGACCCCCCAGGGCGCCATCAACAGCTC | |
| | CCTGCCCTTTCAGAACATCCACCCTGTGACAAT | |
| | CGGGGAGTGCCCCAAGTACGTGAGAAGCACCA | |
| | AGCTGCGGATGGTGACCGGCCTGCGGAACATT | |
| | CCCTCTGTGCAGTCCCGGGGCCTGTTTGGGGCC | |
| | ATCGCCGGCTTCATCGAGGGCGGCTGGACCGG | |
| | CATGATGGACGGCTGGTACGGCTACCACCACC | |
| | AGAACGAACAAGGCTCCGGCTACGCCGCTGAC | |
| | CAAAAGTCCACCCAGAATGCAATCAACGGCAT | |
| | CACCAACAAGGTGAATTCCGTGATCGAGAAGA | |
| | TGAACACCCAGTTCACAGCCGTGGGCAAGGAG | |
| | TTCAACAAGCTGGAAAAGCGGATGGAGAACCT | |
| | GAACAAGAAAGTGGACGACGGCTTCATGGAC | |
| | ATCTGGACCTACAACGCTGAACTGCTGGTGCT | |
| | GCTGGAAAATGAAAGAACACTGGACTTCCACG | |
| | ACTCCAACGTGAAGAACCTGTATGAGAAGGTG | |
| | AAGAATCAGCTCAGAAACAACGCCAAAGAGC | |
| | TGGGCAACGGCTGCTTCGAGTTCTACCACAAG | |
| | TGCGACAACGAGTGTATGGAGTCCGTGAAGAA | |
| | CGGCACATACGACTACCCCAAGTACAGCGAGG | |
| | AGAGCAAGCTGAACAGGGAGAAGATCGATGG | |
| | CGTGAAGCTGGAGTCC | |
| 9 | ADTICIGYHANNSTDTVDTVLEKNVTVTHSVNLL | A/Brisbane/59/2007 |
| | ENSHNGKLCLLKGIAPLQLGNCSVAGWILGNPEC | mature HA Amino Acid |
| | ELLISKESWSYIVEKPNPENGTCYPGHFADYEEL | Sequence |
| | REQLSSVSSFERFEIFPKESSWPNHTVTGVSASCS | |
| | HNGESSFYRNLLWLTGKNGLYPNLSKSYANNKE | |
| | KEVLVLWGVHHPPNIGNQKALYHTENAYVSVV | |
| | SSHYSRKFTPEIAKRPKVRDQEGRINYYWTLLEP | |
| | GDTIIFEANGNLIAPRYAFALSRGFGSGIINSNAP | |
| | MDKCDAKCQTPQGAINSSLPFQNVHPVTIGECPK | |
| | YVRSAKLRMVTGLRNIPSIQSRGLFGAIAGFIEGG | |
| | WTGMVDGWYGYHHQNEQGSGYAADQKSTQN | |
| | AINGITNKVNSVIEKMNTQFTAVGKEFNKLERR | |
| | MENLNKKVDDGFIDIWTYNAELLVLLENERTLD | |
| | FHDSNVKNLYEKVKSQLKNNAKEIGNGCFEFYH | |
| | KCNDECMESVKNGTYDYPKYSEESKLNREKIDG | |
| | VKLES | |
| 10 | GCCGACACCATCTGCATTGGCTATCATGCCAA | A/Brisbane/59/2007 |
| | CAATTCCACAGACACCGTGGACACAGTGCTGG | mature HA nucleic acid |
| | AGAAGAACGTGACCGTGACCCACTCTGTGAAC | sequence |
| | CTGCTGGAGAACTCTCACAACGGCAAACTGTG | |
| | CCTGCTGAAGGGCATCGCCCCTCTGCAGCTCG | |
| | GCAACTGCAGCGTGGCCGGCTGGATCCTGGGC | |
| | AACCCAGAGTGCGAACTGCTGATCTCCAAGGA | |
| | AAGCTGGAGCTACATCGTGGAGAAGCCCAACC | |
| | CCGAGAACGGCACCTGTTACCCTGGCCACTTT | |
| | GCCGACTACGAGGAGCTGCGGGAACAGCTGTC | |
| | TTCCGTGTCCTCCTTCGAAAGATTCGAGATCTT | |
| | TCCCAAGGAGAGCTCCTGGCCCAACCACACAG | |
| | TGACAGGCGTCTCCGCCAGCTGCTCCCACAAT | |
| | GGCGAGTCCTCTTTCTACAGAAACCTGCTGTG | |
| | GCTGACCGGCAAGAACGGCCTGTATCCCAACC | |
| | TGAGCAAGAGCTACGCCAACAACAAGGAGAA | |
| | GGAAGTGCTGGTGCTGTGGGGGGTGCACCACC | |
| | CTCCGAACATCGGCAATCAGAAGGCCCTGTAT | |
| | CACACAGAAAACGCCTACGTCAGCGTGGTGTC | |
| | CTCCCACTACAGCAGAAAGTTCACCCCTGAGA | |
| | TCGCCAAGCGGCCCAAAGTGCGGGATCAGGAG | |
| | GGCCGGATCAATTACTACTGGACCCTGCTGGA | |
| | ACCAGGCGACACAATCATCTTTGAGGCCAACG | |
| | GCAACCTGATCGCCCCAAGATACGCCTTCGCC | |
| | CTGAGCAGAGGCTTCGGCTCTGGCATCATCAA | |
| | CAGCAATGCCCCGATGGACAAGTGCGATGCCA | |
| | AGTGCCAGACCCCACAGGGCGCCATCAACAGC | |
| | AGCCTGCCTTTCCAGAATGTCCACCCCGTGAC | |
| | AATTGGCGAGTGTCCCAAGTACGTGAGAAGCG | |
| | CCAAGCTGCGGATGGTGACCGGCCTGCGGAAC | |
| | ATCCCTTCCATTCAGAGCAGAGGCCTGTTTGGC | |
| | GCCATCGCCGGATTCATTGAAGGCGGCTGGAC | |
| | CGGCATGGTGGACGGCTGGTACGGCTACCACC | |
| | ACCAGAACGAACAGGGCTCTGGCTACGCCGCT | |
| | GACCAAAAGTCCACCCAGAATGCCATCAACGG | |
| | CATCACCAACAAAGTCAACAGCGTGATCGAGA | |
| | AGATGAACACCCAGTTCACCGCTGTGGGCAAA | |
| | GAGTTCAACAAGCTGGAGCGGCGGATGGAAA | |
| | ACCTGAACAAAAAGGTGGACGACGGCTTCATC | |
| | GACATTTGGACATACAACGCAGAGCTGCTGGT | |
| | GCTGCTGGAAAATGAGAGAACCCTGGATTTCC | |
| | ACGACAGCAACGTGAAGAACCTGTACGAGAA | |
| | GGTCAAATCCCAGCTGAAAAACAACGCCAAGG | |
| | AGATCGGCAATGGCTGCTTCGAGTTCTACCAC | |
| | AAGTGCAACGATGAGTGTATGGAGTCCGTGAA | |
| | AAACGGCACCTACGACTACCCGAAGTACAGCG | |
| | AGGAAAGCAAGCTCAATCGGGAGAAGATCGA | |
| | CGGAGTGAAGCTGGAGAGC | |
| 11 | ADTICIGYHANNSTDTVDTVLEKNVTVTHSVNLL | A/Beijing/262/1995 |
| | EDSHNGKLCLLKGIAPLQLGNCSVAGWILGNPEC | mature HA Amino |
| | ESLISKESWSYIVETPNPENGTCYPGYFADYEELR | Acid Sequence |
| | EQLSSVSSFERFEIFPKESSWPNHTVTGVTASCSH | |
| | NGKSSFYRNLLWLTEKNGLYPNLSNSYVNNKEK | |
| | EVLVLWGVHHPSNIRDQRAIYHTENAYVSVVSS | |
| | HYSRRFTPEIAKRPKVRGQEGRINYYWTLLEPGD | |
| | TIIFEANGNLIAPWYAFALSRGFGSGIITSNAPMN | |
| | ECDAKCQTPQGAINSSLPFQNVHPVTIGECPKYV | |
| | RSTKLRMVTGLRNIPSIQSRGLFGAIAGFIEGGWT | |
| | GMMDGWYGYHHQNEQGSGYAADQKSTQNAIN | |
| | GITNKVNSVIEKMNTQFTAVGKEFNKLERRMEN | |
| | LNKKVDDGFLDIWTYNAELLVLLENERTLDFHD | |
| | SNVKNLYEKVKSQLKNNAKEIGNGCFEFYHKCN | |
| | NECMESVKNGTYDYPKYSEESKLNREKIDGVKL | |
| | ES | |
| 12 | GCCGACACCATCTGCATTGGCTACCATGCCAA | A/Beijing/262/1995 |
| | CAACAGCACCGACACCGTGGACACAGTGCTGG | mature HA nucleic acid |
| | AGAAGAACGTGACAGTGACACACTCTGTGAAC | sequence |
| | CTGCTGGAGGACTCCCACAACGGAAAGCTGTG | |
| | CCTGCTGAAGGGAATCGCCCCTCTGCAGCTGG | |
| | GCAACTGTTCCGTCGCCGGCTGGATCCTGGGC | |
| | AATCCTGAGTGCGAGAGCCTGATCTCCAAAGA | |
| | ATCTTGGTCCTACATCGTGGAGACACCCAACC | |
| | CAGAGAACGGCACCTGCTACCCCGGATACTTT | |
| | GCCGACTACGAGGAGCTGCGGGAGCAACTGTC | |
| | CTCAGTGAGCAGCTTCGAAAGATTCGAGATCT | |
| | TCCCCAAAGAGAGCTCTTGGCCCAACCACACA | |
| | GTGACAGGCGTGACAGCCTCCTGCAGCCACAA | |
| | CGGAAAGAGCAGCTTCTACAGAAATCTGCTGT | |
| | GGCTCACCGAGAAGAATGGCCTGTATCCCAAC | |
| | CTCTCCAACAGCTACGTGAACAACAAGGAGAA | |
| | GGAAGTGCTGGTGCTATGGGGGGTGCACCACC | |
| | CCAGCAACATCCGGGACCAGCGGGCCATCTAC | |
| | CACACAGAGAACGCCTACGTCAGCGTGGTGTC | |
| | CTCCCACTACTCTCGGAGATTCACTCCCGAGAT | |
| | CGCCAAGCGGCCCAAGGTGCGGGGCCAGGAG | |
| | GGCCGGATCAATTACTACTGGACCCTGCTGGA | |
| | ACCAGGCGACACCATCATCTTCGAGGCCAACG | |
| | GCAATCTCATCGCCCCCTGGTACGCTTTCGCCC | |
| | TGTCTCGGGGCTTCGGCTCCGGCATCATCACA | |
| | AGCAACGCCCCCATGAATGAGTGCGATGCCAA | |
| | GTGCCAGACACCCCAGGGCGCCATCAACAGCT | |
| | CCCTGCCCTTCCAGAACGTCCACCCTGTGACCA | |
| | TCGGAGAGTGTCCCAAGTACGTGAGATCCACC | |
| | AAGCTGAGAATGGTGACCGGCCTGAGAAACAT | |
| | TCCCAGCATCCAGAGCAGAGGCCTGTTCGGAG | |
| | CCATCGCCGGATTCATTGAGGGAGGATGGACC | |
| | GGCATGATGGACGGCTGGTACGGCTACCACCA | |
| | CCAGAATGAGCAGGGAAGCGGCTACGCCGCTG | |
| | ACCAGAAATCCACACAGAACGCCATCAACGGC | |
| | ATCACCAACAAGGTGAACAGCGTGATCGAAAA | |
| | GATGAACACCCAGTTCACCGCCGTGGGCAAAG | |
| | AGTTCAACAAGCTGGAAAGAAGAATGGAGAA | |
| | CCTGAACAAGAAAGTGGACGACGGCTTCCTGG | |
| | ACATCTGGACATACAACGCAGAGCTGCTGGTG | |
| | CTGCTGGAAAATGAGAGAACACTGGACTTCCA | |
| | CGACAGCAACGTCAAAAACCTGTACGAGAAG | |
| | GTGAAGTCACAGCTGAAAAACAACGCCAAGG | |
| | AGATCGGCAATGGCTGCTTCGAATTCTACCAC | |
| | AAGTGCAACAACGAGTGTATGGAATCCGTGAA | |
| | GAACGGCACCTACGACTACCCGAAGTACTCTG | |
| | AGGAGAGCAAACTGAACAGAGAGAAAATCGA | |
| | CGGGGTGAAGCTGGAGAGC | |
| 13 | ADTICIGYHANNSTDTVDTVLEKNVTVTHSVNLL | A/Puerto Rico/8/1934 |
| | EDSHNGKLCRLKGIAPLQLGKCNIAGWLLGNPE | mature HA Amino |
| | CDPLLPVRSWSYIVETPNSENGICYPGDFIDYEEL | Acid Sequence |
| | REQLSSVSSFERFEIFPKESSWPNHNTNKGVTAA | |
| | CSHEGKSSFYRNLLWLTEKEGSYPKLKNSYVNK | |
| | KGKEVLVLWGIHHPPNSKEQQNLYQNENAYVS | |
| | VVTSNYNRRFTPEIAERPKVRDQAGRMNYYWTL | |
| | LKPGDTIIFEANGNLIAPMYAFALSRGFGSGIITSN | |
| | ASMHECNTKCQTPLGAINSSLPYQNIHPVTIGECP | |
| | KYVRSAKLRMVTGLRNIPSIQSRGLFGAIAGFIEG | |
| | GWTGMIDGWYGYHHQNEQGSGYAADQKSTQN | |
| | AINGITNKVNTVIEKMNIQFTAVGKEFNKLEKRM | |
| | ENLNKKVDDGFLDIWTYNAELLVLLENERTLDF | |
| | HDSNVKNLYEKVKSQLKNNAKEIGNGCFEFYHK | |
| | CDNECMESVRNGTYDYPKYSEESKLNREKVDG | |
| | VKLES | |
| 14 | GCTGACACCATCTGCATTGGCTACCATGCCAA | A/Puerto Rico/8/1934 |
| | CAACTCCACCGACACCGTGGACACAGTGCTGG | mature HA nucleic acid |
| | AGAAGAACGTGACCGTGACACATTCCGTGAAC | sequence |
| | CTGCTGGAGGATTCTCACAACGGCAAGCTCTG | |
| | TCGGCTGAAGGGCATCGCCCCTCTGCAGCTGG | |
| | GCAAGTGCAACATCGCCGGCTGGCTGCTGGGC | |
| | AACCCCGAGTGCGACCCTCTGCTGCCCGTGAG | |
| | ATCCTGGTCCTACATTGTGGAAACCCCTAACA | |
| | GCGAGAACGGCATCTGCTACCCTGGCGACTTC | |
| | ATTGATTACGAGGAGCTGCGGGAGCAGCTGTC | |
| | CTCAGTGAGCAGCTTCGAGAGGTTTGAGATCT | |
| | TCCCCAAGGAGTCCTCTTGGCCCAACCACAAC | |
| | ACAAACAAGGGCGTCACCGCCGCCTGCAGCCA | |
| | TGAGGGAAAGAGCAGCTTCTACAGAAACCTGC | |
| | TGTGGCTGACAGAGAAGGAGGGCTCCTACCCC | |
| | AAGCTGAAGAATTCCTACGTGAACAAGAAAGG | |
| | CAAAGAGGTGCTGGTGCTGTGGGGGATCCACC | |
| | ACCCTCCCAATTCCAAGGAACAGCAGAACCTG | |
| | TACCAGAACGAAAACGCCTACGTCTCTGTGGT | |
| | GACCTCCAACTACAACAGAAGATTCACCCCTG | |
| | AGATCGCAGAGCGGCCCAAGGTGCGGGACCA | |
| | AGCCGGCAGAATGAACTACTACTGGACCCTGC | |
| | TGAAGCCTGGCGACACAATCATCTTCGAGGCC | |
| | AATGGAAACCTGATCGCCCCAATGTACGCCTT | |
| | CGCCCTGTCCCGGGGCTTCGGAAGCGGCATCA | |
| | TCACAAGCAACGCCTCCATGCACGAGTGCAAC | |
| | ACCAAGTGCCAGACCCCACTGGGCGCCATCAA | |
| | TTCCTCTCTCCCTTACCAGAACATCCACCCTGT | |
| | GACCATCGGGGAATGCCCCAAGTACGTGAGAT | |
| | CCGCCAAGCTGCGGATGGTGACCGGCCTGAGA | |
| | AACATCCCCAGCATTCAGAGCAGGGGCCTGTT | |
| | TGGAGCCATCGCTGGCTTCATCGAGGGCGGAT | |
| | GGACCGGCATGATCGACGGCTGGTACGGCTAC | |
| | CACCACCAGAATGAACAGGGCTCCGGCTACGC | |
| | AGCCGATCAGAAAAGCACACAGAATGCCATCA | |
| | ACGGCATCACCAACAAAGTGAACACCGTGATC | |
| | GAAAAGATGAACATCCAGTTCACCGCCGTGGG | |
| | CAAGGAGTTCAACAAGCTGGAGAAGAGAATG | |
| | GAGAACCTGAACAAGAAGGTGGACGACGGCT | |
| | TTCTGGACATTTGGACATACAATGCTGAGCTG | |
| | CTGGTGCTGCTGGAGAATGAGAGGACCCTGGA | |
| | CTTTCACGACTCCAACGTGAAAAACCTGTACG | |
| | AAAAGGTCAAGAGCCAGCTGAAGAACAATGC | |
| | CAAGGAGATCGGCAACGGCTGCTTCGAGTTCT | |
| | ACCACAAGTGCGACAACGAGTGTATGGAGTCC | |
| | GTGAGAAACGGCACCTATGACTACCCCAAGTA | |
| | CTCTGAGGAGTCCAAACTGAACAGAGAGAAG | |
| | GTGGACGGAGTGAAGCTGGAGTCT | |
| 15 | DTICIGYHANNSTDTVDTVLEKNVTVTHSVNLLE | A/New_York/1/1918 |
| | DSHNGKLCKLKGIAPLQLGKCNIAGWLLGNPEC | mature HA Amino |
| | DLLLTASSWSYIVETSNSENGTCYPGDFIDYEELR | Acid Sequence |
| | EQLSSVSSFEKFEIFPKTSSWPNHETTKGVTAACS | |
| | YAGASSFYRNLLWLTKKGSSYPKLSKSYVNNKG | |
| | KEVLVLWGVHHPPTGTDQQSLYQNADAYVSVG | |
| | SSKYNRRFTPEIAARPKVRDQAGRMNYYWTLLE | |
| | PGDTITFEATGNLIAPWYAFALNRGSGSGIITSDA | |
| | PVHDCNTKCQTPHGAINSSLPFQNIHPVTIGECPK | |
| | YVRSTKLRMATGLRNIPSIQSRGLFGAIAGFIEGG | |
| | WTGMIDGWYGYHHQNEQGSGYAADRKSTQNAI | |
| | DGITNKVNSVIEKMNTQFTSVGKEFNHLEKRIEN | |
| | LNRKVDDGFLDVWTYNAELLVLLENERTLDYH | |
| | DSNVKNLYEKVRSQLKNNAKEIGNGCFEFYHKC | |
| | DDSCMESVKNGTYDYPKYSEESKLNREEIDGVK | |
| | LES | |
| 16 | GACACAATCTGCATTGGCTACCATGCCAACAA | A/New_York/1/1918 |
| | CAGCACAGACACCGTGGACACCGTCCTGGAGA | mature HA nucleic acid |
| | AAAACGTGACAGTGACCCACAGCGTGAACCTG | sequence |
| | CTGGAAGACTCCCACAATGGAAAACTGTGCAA | |
| | GCTGAAGGGCATCGCCCCTCTGCAGCTGGGCA | |
| | AGTGCAACATCGCCGGCTGGCTGCTGGGCAAC | |
| | CCAGAGTGCGACCTGCTGCTGACAGCCAGTAG | |
| | CTGGAGCTACATCGTGGAAACCAGCAACAGCG | |
| | AAAATGGCACATGCTACCCTGGCGACTTCATC | |
| | GATTACGAGGAGCTGCGGGAGCAGCTGAGCA | |
| | GCGTCAGCTCTTTCGAGAAGTTTGAGATCTTCC | |
| | CCAAAACCTCAAGCTGGCCCAACCACGAAACC | |
| | ACAAAGGGCGTGACCGCCGCCTGCAGCTACGC | |
| | AGGCGCCAGCTCTTTCTACAGAAACCTGCTGT | |
| | GGCTGACAAAGAAGGGGAGCAGCTACCCCAA | |
| | GCTCTCCAAAAGCTACGTGAACAACAAGGGCA | |
| | AGGAGGTGCTGGTGCTATGGGGGGTGCATCAC | |
| | CCTCCAACCGGCACCGACCAGCAGAGCCTGTA | |
| | CCAGAACGCCGACGCCTACGTCTCCGTGGGCT | |
| | CCTCCAAGTACAACAGAAGATTCACCCCTGAG | |
| | ATCGCTGCTCGGCCCAAGGTGCGGGACCAGGC | |
| | CGGAAGGATGAACTACTACTGGACCCTGCTGG | |
| | AGCCTGGCGACACAATCACCTTCGAGGCCACA | |
| | GGCAACCTGATCGCCCCTTGGTACGCCTTCGCT | |
| | CTCCGACGCCCCTGTGCACGACTGCAACACAA | |
| | CTCAACAGAGGCTCCGGCAGCGGCATCATCAC | |
| | AGTGCCAGACCCCTCACGGGGCCATCAACTCC | |
| | AGCCTGCCTTTCCAGAACATCCACCCTGTGACC | |
| | ATCGGAGAATGCCCCAAGTACGTGAGAAGCAC | |
| | CAAGCTGAGAATGGCCACAGGCCTGAGAAAC | |
| | ATCCCTTCCATCCAGAGCAGGGGCCTGTTTGG | |
| | GGCCATCGCCGGCTTCATCGAGGGCGGCTGGA | |
| | CCGGCATGATCGACGGCTGGTACGGCTACCAC | |
| | CATCAGAACGAACAGGGCTCCGGCTACGCCGC | |
| | CGACAGAAAGTCCACCCAGAATGCCATTGACG | |
| | GCATCACCAACAAGGTGAATTCCGTGATCGAG | |
| | AAGATGAACACCCAGTTCACCAGCGTGGGAAA | |
| | AGAGTTCAACCACCTGGAGAAGAGAATCGAG | |
| | AACCTGAACAGGAAGGTGGATGACGGCTTCCT | |
| | GGACGTGTGGACATACAACGCTGAACTGCTGG | |
| | TGCTGCTGGAAAACGAAAGAACCCTGGACTAC | |
| | CACGACAGCAACGTGAAGAACCTGTACGAGA | |
| | AGGTGCGGAGCCAGCTGAAGAACAACGCCAA | |
| | GGAAATCGGCAATGGCTGTTTCGAATTCTACC | |
| | ACAAGTGCGACGACAGCTGTATGGAATCCGTG | |
| | AAAAACGGCACATACGACTACCCCAAGTACAG | |
| | CGAGGAGAGCAAGCTGAACAGAGAGGAAATC | |
| | GATGGCGTGAAACTGGAGTCC | |
| 17 | ADTLCIGYHANNSTDTVDTVLEKNVTVTHSVNL | A/Wisconsin/28/2011. |
| | LENRHNGKLCKLRGVAPLHLGKCNIAGWLLGNP | 2011/12 mature HA |
| | ECESLSTASSWSYIVETSNSDNGTCYPGDFINYEE | Amino Acid Sequence |
| | LREQLSSVSSFERFEIFPKTSSWPNHDTNRGVTAA | |
| | CPHDGTNSFYRNLIWLVKKGNSYPKINKSYINNK | |
| | EKEILVLWAIHHPSTSADQQSLYQNADAYVFVG | |
| | SSRYSRKFEPEVATRPKVRDQAGRMNYYWTLVE | |
| | PGDKITFEATGNLVVPRYAFALKRNSGSGIIISDT | |
| | SVHDCDTNCQTPNGAINTSLPFQNIHPVTIGECPK | |
| | YVKSTKLRMATGLRNIPSIQSRGLFGAIAGFIEGG | |
| | WTGMIDGWYGYHHQNEQGSGYAADLKSTQNAI | |
| | DGITNKVNSVIEKMNTQFTAVGKEFSHLERRIEN | |
| | LNKKVDDGFLDIWTYNAELLVLLENERTLDYHD | |
| | SNVKNLYEKVRSQLKNNAKEIGNGCFEFYHKCD | |
| | DMCMESVKNGTYDYPKYSEEAKLNREEIDGVKL | |
| | ES | |
| 18 | GCTGACACCCTGTGCATTGGCTACCATGCCAA | A/Wisconsin/28/2011. |
| | CAACAGCACCGACACCGTCGACACAGTCCTGG | 2011/12 mature HA |
| | AAAAGAACGTGACAGTGACCCACTCCGTGAAT | nucleic acid sequence |
| | CTGCTGGAAAACAGACACAACGGCAAACTGTG | |
| | CAAGCTGCGGGGCGTGGCCCCTCTGCACCTGG | |
| | GCAAATGCAACATCGCCGGATGGCTGCTGGGC | |
| | AACCCAGAGTGCGAAAGCCTGAGCACCGCCAG | |
| | CTCTTGGTCCTACATCGTCGAAACCAGCAACA | |
| | GCGACAATGGCACATGCTACCCTGGCGACTTC | |
| | ATCAACTACGAGGAACTGAGAGAGCAGCTGA | |
| | GCTCTGTGTCCTCCTTCGAGAGGTTTGAGATCT | |
| | TCCCCAAGACCTCAAGCTGGCCCAATCACGAC | |
| | ACAAACAGAGGAGTGACCGCCGCCTGTCCTCA | |
| | CGATGGAACCAACAGCTTCTACAGAAATCTGA | |
| | TCTGGCTCGTGAAGAAGGGCAACTCTTACCCC | |
| | AAGATCAACAAGTCTTACATCAACAACAAAGA | |
| | GAAGGAAATTCTGGTGCTGTGGGCCATCCACC | |
| | ACCCCAGCACCTCCGCTGACCAGCAAAGCCTG | |
| | TACCAGAACGCTGACGCCTACGTCTTCGTGGG | |
| | CAGCTCCCGGTACAGCAGAAAGTTCGAACCTG | |
| | AAGTGGCCACCCGGCCCAAGGTGCGGGACCAG | |
| | GCCGGCAGAATGAACTACTACTGGACCCTGGT | |
| | CGAACCTGGCGACAAGATCACCTTCGAAGCCA | |
| | CCGGAAACCTGGTGGTGCCCAGATACGCCTTC | |
| | GCCCTGAAGAGAAATTCGGGCAGCGGCATCAT | |
| | CATCAGCGACACATCCGTCCACGACTGCGACA | |
| | CCAACTGTCAGACCCCAAATGGGGCCATCAAC | |
| | ACCAGCCTGCCTTTCCAGAACATCCACCCTGTC | |
| | ACCATCGGAGAGTGCCCCAAGTACGTGAAGTC | |
| | CACAAAGCTGAGAATGGCCACCGGCCTGCGGA | |
| | ACATCCCCAGCATCCAGAGCCGGGGACTGTTT | |
| | GGCGCCATCGCCGGCTTCATCGAGGGCGGCTG | |
| | GACCGGCATGATCGACGGCTGGTACGGCTACC | |
| | ACCATCAGAATGAGCAGGGCAGCGGCTACGCC | |
| | GCTGACCTGAAGTCCACACAGAACGCCATCGA | |
| | CGGCATCACCAACAAGGTGAACAGCGTGATCG | |
| | AGAAAATGAACACCCAGTTCACCGCCGTGGGC | |
| | AAGGAATTCTCCCACCTGGAGAGACGGATTGA | |
| | GAACCTGAACAAGAAGGTGGACGACGGCTTTC | |
| | TGGACATCTGGACCTACAACGCTGAACTGCTG | |
| | GTGCTGCTGGAAAATGAGAGAACACTGGACTA | |
| | CCACGATTCCAATGTCAAAAACCTGTATGAGA | |
| | AAGTGCGGAGCCAGCTGAAGAACAATGCCAA | |
| | GGAAATTGGCAACGGCTGTTTCGAGTTCTACC | |
| | ACAAGTGCGATGATATGTGTATGGAGTCCGTG | |
| | AAGAATGGAACATACGACTACCCCAAGTACAG | |
| | CGAAGAGGCCAAGCTGAACAGAGAGGAGATC | |
| | GACGGGGTGAAGCTGGAGTCT | |
| 19 | TATLCLGHHAVPNGTIVKTITNDRIEVTNATELV | A/Indiana/11/2018 |
| | QNSSIGEICDSPHQILDGENCTLIDALLGDPQCDG | mature HA Amino |
| | FQNKKWDLFVERSKAYSNCYPYDVPDYASLRSL | Acid Sequence |
| | VASSGTLEFNNESFNWTGVKQNGTSSACIRKSSS | |
| | SFFSRLNWLTHLNYTYPALNVTMPNNEQFDKLY | |
| | IWGVHHPGTDKDQIFLYAQSSGRITVSTKRSQQA | |
| | VIPNIGSRPRIRDIPSRISIYWTIVKPGDILLINSTGN | |
| | LIAPRGYFKIQSGKSSIMRSDAPIGKCKSECITPNG | |
| | SIPNDKPFQNVNRITYGACPRYVKHSTLKLATGM | |
| | RNVPEKQTRGIFGAIAGFIENGWEGMVDGWYGF | |
| | RHQNSEGRGQAADLKSTQAAIDQINGKLNRLIG | |
| | KTNEKFHQIEKEFSEVEGRIQDLEKYVEDTKIDL | |
| | WSYNAELLVALENQHTIDLTDSEMNKLFEKTKK | |
| | QLRENAEDMGNGCFKIYHKCDNACIGSIRNGTY | |
| | DHNVYRDEALNNRFQIKGVELKS | |
| 20 | ACCGCCACCCTGTGCCTGGGGCACCACGCTGT | A/Indiana/11/2018 |
| | GCCAAATGGCACCATTGTGAAGACCATTACCA | mature HA nucleic acid |
| | ATGACAGAATCGAGGTGACCAATGCCACCGAA | sequence |
| | CTGGTGCAGAATTCCAGCATTGGCGAGATCTG | |
| | CGACAGCCCTCACCAGATCCTGGATGGCGAGA | |
| | ACTGCACCCTGATCGATGCTCTGCTGGGCGAC | |
| | CCTCAGTGCGATGGCTTCCAGAACAAGAAATG | |
| | GGACCTGTTCGTGGAGCGGAGCAAGGCCTACA | |
| | GCAACTGCTACCCCTACGACGTCCCCGATTAC | |
| | GCCAGCCTGCGGAGCCTGGTGGCCTCCTCCGG | |
| | CACACTGGAGTTCAACAACGAAAGCTTCAACT | |
| | GGACAGGAGTGAAGCAGAACGGCACATCCAG | |
| | CGCCTGCATCAGAAAAAGCTCCAGCAGCTTCT | |
| | TCTCTCGACTGAACTGGCTGACCCACCTGAACT | |
| | ACACCTACCCCGCCCTGAACGTGACCATGCCA | |
| | AACAATGAGCAGTTTGACAAACTGTACATTTG | |
| | GGGAGTGCACCACCCCGGCACAGACAAGGAC | |
| | CAGATCTTTCTGTACGCCCAGAGCAGCGGCAG | |
| | GATCACCGTGTCTACCAAGAGAAGCCAGCAGG | |
| | CCGTGATCCCCAACATCGGCAGCAGACCTCGG | |
| | ATCAGAGACATCCCCTCCAGAATCTCCATCTA | |
| | CTGGACAATTGTGAAGCCTGGAGACATTCTGC | |
| | TGATCAACAGCACCGGCAACCTGATCGCCCCT | |
| | CGAGGATACTTCAAGATCCAGAGCGGCAAAAG | |
| | CAGCATCATGAGATCTGACGCCCCCATCGGCA | |
| | AATGCAAAAGCGAGTGTATCACCCCCAACGGC | |
| | AGCATCCCCAACGACAAGCCCTTTCAGAACGT | |
| | GAACAGAATCACCTATGGCGCCTGTCCCAGGT | |
| | ACGTGAAGCACAGCACCCTGAAGCTGGCCACA | |
| | GGAATGAGAAACGTCCCCGAGAAGCAAACCC | |
| | GGGGCATCTTTGGCGCTATTGCCGGCTTCATCG | |
| | AGAACGGCTGGGAAGGGATGGTGGACGGCTG | |
| | GTACGGCTTCCGGCACCAGAACAGCGAGGGAA | |
| | GAGGCCAGGCCGCTGACCTGAAGTCCACCCAG | |
| | GCCGCCATTGATCAGATCAACGGCAAGCTGAA | |
| | CAGACTGATCGGCAAGACCAACGAAAAATTCC | |
| | ACCAGATCGAAAAGGAATTCAGCGAAGTGGA | |
| | GGGCAGAATTCAGGACCTGGAAAAGTACGTGG | |
| | AGGACACAAAGATCGACCTGTGGTCCTACAAC | |
| | GCTGAACTGCTGGTGGCCCTGGAGAATCAGCA | |
| | CACCATCGACCTGACCGACAGCGAGATGAACA | |
| | AACTGTTTGAAAAGACCAAGAAGCAGCTGAGA | |
| | GAAAACGCCGAGGACATGGGCAATGGATGCTT | |
| | CAAAATCTACCACAAGTGCGACAACGCCTGTA | |
| | TTGGCAGCATCAGAAATGGCACCTACGACCAC | |
| | AACGTCTACAGGGACGAGGCCCTGAACAACAG | |
| | ATTTCAAATCAAGGGCGTGGAGCTGAAGTCT | |
| 21 | TATLCLGHHAVPNGTIVKTITNDRIEVTNATELV | A/California/07/2004 |
| | QNSSIGEICDSPHQILDGENCTLIDALLGDPQCDG | mature HA Amino |
| | FQNKKWDLFVERSKAYSNCYPYDVPDYASLRSL | Acid Sequence |
| | VASSGTLEFNNESFNWTGVKQNGTSSACIRKSSS | |
| | SFFSRLNWLTHLNYTYPALNVTMPNNEQFDKLY | |
| | IWGVHHPGTDKDQIFLYAQSSGRITVSTKRSQQA | |
| | VIPNIGSRPRIRDIPSRISIYWTIVKPGDILLINSTGN | |
| | LIAPRGYFKIQSGKSSIMRSDAPIGKCKSECITPNG | |
| | SIPNDKPFQNVNRITYGACPRYVKHSTLKLATGM | |
| | RNVPEKQTRGIFGAIAGFIENGWEGMVDGWYGF | |
| | RHQNSEGRGQAADLKSTQAAIDQINGKLNRLIG | |
| | KTNEKFHQIEKEFSEVEGRIQDLEKYVEDTKIDL | |
| | WSYNAELLVALENQHTIDLTDSEMNKLFEKTKK | |
| | QLRENAEDMGNGCFKIYHKCDNACIGSIRNGTY | |
| | DHNVYRDEALNNRFQIKGVELKS | |
| 22 | ACAGCCACACTCTGTCTGGGCCATCACGCTGT | A/California/07/2004 |
| | GCCAAACGGCACAATCGTGAAGACAATCACCA | mature HA nucleic acid |
| | ACGACCAGATTGAGGTGACAAATGCCACAGAG | sequence |
| | CTGGTGCAGAGCTCCAGCACAGGCGGCATCTG | |
| | CGACAGCCCTCATCAGATCCTGGATGGCGAGA | |
| | ACTGCACACTGATCGATGCCCTGCTGGGCGAC | |
| | CCTCAGTGCGACGGCTTCCAGAACAAGAAATG | |
| | GGACCTGTTCGTGGAGCGGAGCAAGGCTTACA | |
| | GCAACTGTTACCCCTACGACGTCCCTGACTAC | |
| | GCCTCCCTGCGGAGCCTGGTGGCCTCCTCCGG | |
| | CACCCTGGAGTTTAACAATGAATCCTTTAACTG | |
| | GACAGGCGTCACCCAGAACGGCACATCTTCCA | |
| | GCTGCAAGAGAAGAAGCAACAACTCCTTCTTC | |
| | AGCAGACTGAATTGGCTGACCCACCTGAAGTT | |
| | CAAGTACCCTGCCCTGAACGTGACCATGCCAA | |
| | ACAATGAGAAGTTCGACAAGCTGTACATCTGG | |
| | GGGGTTCACCACCCTGGCACAAACAACGACCA | |
| | GATCAGCCTGTACACCCAGGCCAGCGGCCGGA | |
| | TCACCGTGTCTACAAAGCGGAGCCAGCAGACA | |
| | GTGATCCCCAACATCGGCTCCAGACCTCGGGT | |
| | GAGAGACATCCCCTCCAGAATCAGCATCTACT | |
| | GGACCATCGTGAAGCCAGGCGACATCCTGCTC | |
| | ATCAACAGCACCGGAAACCTGATTGCACCCAG | |
| | AGGCTACTTCAAGATCAGAAGCGGCAAGAGCA | |
| | GCATCATGAGAAGCGATGCCCCCATCGGCAAG | |
| | TGCAACAGCGAGTGTATCACCCCCAACGGCAG | |
| | CATCCCCAATGACAAGCCCTTCCAGAACGTGA | |
| | ACAGAATCACCTATGGCGCCTGCCCCAGGTAC | |
| | GTCAAGCAGAACACCCTGAAGCTGGCCACCGG | |
| | AATGAGAAACGTCCCCGAGAAGCAGACCAGG | |
| | GGGATCTTTGGAGCCATCGCCGGCTTCATCGA | |
| | GAACGGCTGGGAGGGAATGGTGGACGGATGG | |
| | TACGGCTTCCGGCACCAGAACTCTGAGGGCAT | |
| | TGGCCAGGCCGCAGATCTGAAGTCCACCCAGG | |
| | CCGCCATCAACCAGATCAACGGCAAGCTGAAC | |
| | AGACTGATCGGCAAGACAAATGAAAAATTCCA | |
| | CCAGATCGAGAAAGAATTCAGCGAGGTGGAA | |
| | GGCCGGATTCAGGACCTGGAGAAGTACGTGGA | |
| | GGACACCAAGATCGACCTGTGGAGCTACAACG | |
| | CTGAACTGCTCGTGGCCCTGGAGAACCAGCAC | |
| | ACAATCGACCTGACAGACAGCGAGATGAACA | |
| | AGCTGTTCGAAAGAACAAAGAAACAGCTGAG | |
| | AGAAAACGCCGAGGACATGGGCAACGGCTGC | |
| | TTCAAGATCTACCACAAGTGCGACAACGCCTG | |
| | CATTGGCAGCATCAGAAACGGCACCTACGACC | |
| | ATGACGTTTACCGGGACGAGGCCCTGAACAAC | |
| | CGGTTCCAGATCAAGGGCGTGGAACTGAAGTC | |
| | C | |
| 23 | TATLCLGHHAVPNGTIVKTITNDRIEVTNATELV | A/Alaska/01/2021 |
| | QNSSIGEICNSPHQILDGGNCTLIDALLGDPQCDG | mature HA Amino |
| | FQNKEWDLFVERSRANSSCYPYDVPDYASLRSL | Acid Sequence |
| | VASSGTLEFKNESFNWTGVKQNGTSSACIRGSSS | |
| | SFFSRLNWLTSLNNIYPAQNVTMPNKEQFDKLYI | |
| | WGVHHPDTDKNQFSLFAQSSGRITVSTKRSQQA | |
| | VIPNIGSRPRIRDIPSRISIYWTIVKPGDILLINSTGN | |
| | LIAPRGYFKIRSGKSSIMRSDAPIGKCKSECITPNG | |
| | SIPNDKPFQNVNRITYGACPRYVKQSTLKLATGM | |
| | RNVPEKQTRGIFGAIAGFIENGWEGMVDGWYGF | |
| | RHQNSEGRGQAADLKSTQAAIDQISGKLNRLIGK | |
| | TNEKFHQIEKEFSEVEGRVQDLEKYVEDTKIDLW | |
| | SYNAELLVALENQHTIDLTDSEMNKLFEKTKKQ | |
| | LRENAEDMGNGCFKIYHKCDNACIGSIRNETYD | |
| | HNVYRDEALNNRFQIKGVELKS | |
| 24 | ACAGCCACACTGTGCCTGGGCCATCACGCTGT | A/Alaska/01/2021 |
| | GCCCAACGGAACCATCGTGAAGACCATTACCA | mature HA nucleic acid |
| | ACGACAGAATCGAAGTGACAAATGCCACAGA | sequence |
| | GCTGGTGCAGAACAGCAGCATTGGCGAGATCT | |
| | GCAACAGCCCTCACCAGATCCTGGACGGAGGC | |
| | AACTGCACACTGATCGATGCCCTGCTCGGCGA | |
| | CCCTCAGTGCGATGGCTTCCAGAACAAGGAGT | |
| | GGGACCTGTTCGTGGAAAGAAGCAGAGCCAAC | |
| | AGCTCCTGCTACCCCTACGACGTCCCTGACTAC | |
| | GCCAGCCTGCGGAGCCTGGTGGCCTCCAGCGG | |
| | CACCCTGGAGTTCAAAAACGAAAGCTTCAATT | |
| | GGACCGGAGTGAAACAGAACGGCACCTCCAG | |
| | CGCCTGTATCAGAGGCTCCAGCAGCTCTTTCTT | |
| | CAGCAGACTGAACTGGCTGACAAGCCTGAACA | |
| | ACATTTACCCCGCCCAGAACGTGACAATGCCC | |
| | AACAAGGAGCAGTTTGACAAACTGTACATCTG | |
| | GGGAGTGCACCACCCTGATACCGACAAAAACC | |
| | AGTTCTCCCTGTTCGCCCAGTCTTCCGGCAGAA | |
| | TCACCGTGTCTACCAAGCGGAGCCAGCAGGCC | |
| | GTGATCCCCAACATCGGCAGCCGGCCCCGGAT | |
| | TCGGGACATCCCTTCCCGGATCAGCATTTACTG | |
| | GACCATCGTGAAGCCTGGAGACATTCTCCTGA | |
| | TCAACAGCACCGGCAACCTGATCGCCCCTCGG | |
| | GGCTACTTCAAGATCAGAAGCGGCAAGTCCAG | |
| | CATCATGAGATCTGACGCCCCTATCGGCAAGT | |
| | GCAAGTCTGAGTGTATCACACCCAACGGCAGC | |
| | ATCCCCAACGACAAGCCCTTTCAGAACGTGAA | |
| | CAGAATCACCTATGGCGCCTGTCCCAGGTACG | |
| | TGAAGCAGAGCACCCTGAAGCTGGCCACAGGG | |
| | ATGAGAAACGTCCCCGAAAAGCAGACCCGGG | |
| | GCATCTTTGGCGCTATCGCCGGCTTCATCGAGA | |
| | ACGGCTGGGAGGGCATGGTGGACGGCTGGTAC | |
| | GGCTTCCGGCACCAGAACAGCGAGGGCAGAG | |
| | GCCAGGCCGCTGACCTGAAGTCCACCCAGGCT | |
| | GCCATCGACCAGATCAGCGGAAAGCTGAACAG | |
| | ACTGATCGGCAAAACAAACGAAAAGTTCCATC | |
| | AGATCGAGAAGGAGTTCTCCGAGGTGGAAGGC | |
| | CGGGTGCAGGACCTGGAAAAGTACGTGGAAG | |
| | ACACCAAGATCGACCTGTGGTCCTACAACGCT | |
| | GAACTGCTGGTCGCCCTGGAGAATCAGCACAC | |
| | CATCGACCTGACCGACAGCGAGATGAACAAAC | |
| | TGTTCGAGAAGACCAAGAAGCAGCTGCGGGA | |
| | AAACGCCGAGGATATGGGCAACGGATGCTTCA | |
| | AGATTTACCACAAGTGCGACAACGCCTGCATT | |
| | GGCAGCATCAGAAATGAGACATACGACCACA | |
| | ATGTTTACCGGGATGAGGCCCTGAACAACAGA | |
| | TTCCAGATCAAGGGAGTGGAGCTGAAGTCC | |
| 25 | TATLCLGHHAVPNGTIVKTITNDRIEVTNATELV | A/Cambodia/e0826360/ |
| | QNSSIGEICDSPHQILDGGNCTLIDALLGDPQCDG | 2020 mature HA |
| | FQNKEWDLFVERSRANSNCYPYDVPDYASLRSL | Amino Acid Sequence |
| | VASSGTLEFKNESFNWTGVKQNGTSSACIRGSSS | |
| | SFFSRLNWLTHLNYTYPALNVTMPNNEQFDKLY | |
| | IWGVHHPSTDKDQISLFAQPSGRITVSTKRSQQA | |
| | VIPNIGSRPRIRDIPSRISIYWTIVKPGDILLINSTGN | |
| | LIAPRGYFKIRSGKSSIMRSDAPIGKCKSECITPNG | |
| | SIPNDKPFQNVNRITYGACPRYVKQSTLKLATGM | |
| | RNVPEKQTRGIFGAIAGFIENGWEGMVDGWYGF | |
| | RHQNSEGRGQAADLKSTQAAIDQINGKLNRLIG | |
| | KTNEKFHQIEKEFSEVEGRVQDLEKYVEDTKIDL | |
| | WSYNAELLVALENQHTIDLTDSEMNKLFEKTKK | |
| | QLRENAEDMGNGCFKIYHKCDNACIGSIRNETY | |
| | DHNVYRDEALNNRFQIKGVELKS | |
| 26 | ACCGCCACACTGTGCCTGGGCCATCACGCTGT | A/Cambodia/e0826360/ |
| | GCCAAACGGCACCATCGTGAAGACCATCACCA | 2020 mature HA |
| | ACGATAGAATCGAGGTGACCAATGCCACAGAA | nucleic acid sequence |
| | CTGGTGCAGAATAGCAGCATTGGCGAGATCTG | |
| | CGACTCCCCCCACCAGATCCTGGACGGAGGAA | |
| | ACTGCACACTGATCGATGCCCTGCTCGGCGAC | |
| | CCTCAGTGCGATGGCTTTCAGAACAAGGAGTG | |
| | GGACCTGTTCGTGGAGCGGAGCAGAGCCAACA | |
| | GCAACTGTTACCCCTACGACGTCCCTGACTAC | |
| | GCCAGCCTGCGGAGCCTGGTGGCCTCCAGCGG | |
| | CACACTGGAGTTCAAGAATGAGAGCTTCAACT | |
| | GGACCGGAGTGAAACAGAACGGCACAAGCAG | |
| | CGCCTGCATCAGAGGCTCCAGCTCATCCTTCTT | |
| | CAGCAGACTGAACTGGCTGACCCACCTGAACT | |
| | ACACCTACCCTGCCCTGAACGTGACAATGCCA | |
| | AACAATGAACAGTTTGACAAGCTGTACATCTG | |
| | GGGAGTGCACCACCCTTCCACCGACAAAGATC | |
| | AGATTTCTCTGTTCGCTCAGCCAAGCGGCAGA | |
| | ATCACCGTGTCTACCAAGCGGAGCCAGCAGGC | |
| | CGTGATTCCTAACATCGGCAGCAGACCTCGGA | |
| | TCAGAGACATCCCCAGCAGAATCTCAATCTAC | |
| | TGGACAATCGTGAAGCCTGGCGACATCCTGCT | |
| | GATCAATTCCACAGGAAACCTGATTGCACCTC | |
| | GGGGCTACTTCAAGATCAGGAGCGGCAAGAGC | |
| | AGCATCATGAGAAGCGATGCTCCCATCGGCAA | |
| | GTGCAAGAGCGAGTGTATCACCCCCAACGGCA | |
| | GCATCCCCAATGACAAGCCTTTTCAGAACGTG | |
| | AACAGAATCACCTATGGCGCCTGCCCCAGGTA | |
| | CGTCAAGCAGTCAACACTGAAGCTGGCCACAG | |
| | GGATGAGAAACGTCCCTGAGAAGCAAACACG | |
| | GGGAATCTTTGGCGCTATCGCCGGCTTCATCG | |
| | AGAATGGCTGGGAAGGCATGGTGGACGGCTG | |
| | GTACGGCTTCCGGCATCAGAACAGCGAGGGAA | |
| | GAGGCCAGGCCGCTGACCTGAAGTCAACACAG | |
| | GCCGCCATCGACCAGATCAATGGCAAGCTGAA | |
| | CAGGCTGATCGGCAAGACAAACGAAAAGTTTC | |
| | ACCAGATCGAGAAGGAGTTCAGCGAAGTGGA | |
| | GGGCAGAGTGCAGGACCTGGAGAAGTACGTG | |
| | GAGGATACCAAGATCGACCTGTGGTCCTACAA | |
| | CGCAGAGCTGCTCGTGGCCCTGGAGAACCAGC | |
| | ACACCATTGACCTGACCGACAGCGAGATGAAC | |
| | AAGCTGTTCGAGAAGACCAAGAAACAGCTGA | |
| | GAGAGAATGCCGAGGACATGGGCAACGGCTG | |
| | CTTCAAGATCTACCACAAGTGCGACAACGCCT | |
| | GCATTGGAAGCATCAGAAACGAAACCTACGAT | |
| | CACAACGTCTACAGAGACGAGGCTCTGAACAA | |
| | CCGGTTCCAGATCAAGGGCGTGGAGCTGAAGT | |
| | CC | |
| 27 | TATLCLGHHAVPNGTLVKTITNDQIEVTNATELV | A/Nanchang/933/1995 |
| | QSSSTGRICDSPHRILDGKNCTLIDALLGDPHCDG | mature HA Amino |
| | FQNKEWDLFVERSKAYSNCYPYDVPDYASLRSL | Acid Sequence |
| | VASSGTLEFTNEGENWTGVAQDGTSYACKRGSV | |
| | KSFFSRLNWLHKLEYKYPALNVTMPNNDKFDKL | |
| | YIWGVHHPSTDSDQTSLYVQASGRVTVSTKRSQ | |
| | QTVIPNIGSRPWVRGISSRISIYWTIVKPGDILLIKS | |
| | TGNLIAPRGYFKIRSGKSSIMRSDAPIGNCNSECIT | |
| | PNGSIPNDKPFQNVNRITYGACPRYVKQNTLKLA | |
| | TGMRNVPEKQTRGIFGAIAGFIENGWEGMVDGW | |
| | YGFRHQNSEGTGQAADLKSTQAAINQINGKLNR | |
| | LIEKTNEKFHQIEKEFSEVEGRIQDLEKYVEDTKI | |
| | DLWSYNAELLVALENQHTIDLTDSEMNKLFERT | |
| | RKQLRENAEDMGNGCFKIYHKCDNACIGSIRNG | |
| | TYDHDVYRDEALNNRFQIKGVELKS | |
| 28 | ACAGCCACCCTGTGCCTGGGACACCACGCTGT | A/Nanchang/933/1995 |
| | GCCAAATGGCACCCTGGTGAAGACAATCACCA | mature HA nucleic acid |
| | ACGACCAGATCGAAGTGACAAATGCCACAGA | sequence |
| | GCTGGTGCAGAGCTCCTCCACCGGCAGAATCT | |
| | GCGACTCTCCTCACAGAATCCTGGACGGCAAG | |
| | AACTGCACCCTGATCGATGCCCTGCTGGGCGA | |
| | CCCTCACTGCGACGGCTTCCAGAACAAGGAGT | |
| | GGGACCTGTTCGTGGAGAGAAGCAAGGCCTAC | |
| | TCCAACTGTTACCCTTACGACGTCCCTGATTAC | |
| | GCCTCCCTGAGAAGCCTGGTGGCCTCCTCTGG | |
| | CACCCTGGAGTTCACCAATGAGGGCTTCAACT | |
| | GGACCGGAGTGGCCCAGGATGGCACCTCCTAC | |
| | GCCTGCAAGAGAGGCTCCGTGAAAAGCTTCTT | |
| | CAGCAGACTGAACTGGCTCCACAAGCTGGAGT | |
| | ACAAGTACCCCGCCCTGAACGTGACCATGCCA | |
| | AACAACGACAAGTTCGACAAGCTCTACATCTG | |
| | GGGCGTCCACCACCCCAGCACCGACAGCGACC | |
| | AGACATCTCTCTATGTCCAGGCCAGCGGCAGA | |
| | GTGACAGTCAGCACCAAGAGAAGCCAGCAAA | |
| | CAGTGATCCCCAACATCGGCAGCAGACCTTGG | |
| | GTGCGGGGCATCTCTTCCAGAATCAGCATCTA | |
| | CTGGACAATCGTGAAGCCTGGCGATATCCTGC | |
| | GCTCCATCATGAGAAGCGATGCTCCTATCGGC | |
| | TGATCAAGAGCACCGGCAACCTGATCGCCCCC | |
| | AACTGCAACTCTGAGTGTATCACCCCCAACGG | |
| | AGAGGCTACTTCAAGATCAGAAGCGGAAAGA | |
| | CTCCATTCCCAACGACAAGCCTTTTCAGAACGT | |
| | GAACAGAATCACCTACGGGGCCTGCCCCAGGT | |
| | ACGTGAAGCAGAACACCCTGAAGCTGGCCACC | |
| | GGAATGAGAAACGTCCCCGAGAAGCAGACAC | |
| | GGGGCATCTTTGGCGCCATCGCCGGCTTCATC | |
| | GAGAACGGCTGGGAGGGGATGGTGGATGGCT | |
| | GGTACGGCTTCCGGCACCAGAATTCGGAGGGC | |
| | ACCGGCCAGGCCGCCGACTTGAAATCCACCCA | |
| | GGCCGCCATCAACCAGATCAACGGCAAGCTGA | |
| | ACAGACTGATCGAGAAGACAAATGAGAAGTTC | |
| | CACCAGATCGAGAAGGAGTTCAGCGAAGTGG | |
| | AGGGAAGAATCCAGGACCTGGAGAAGTACGT | |
| | GGAAGACACCAAGATCGACCTGTGGTCCTACA | |
| | ACGCAGAACTGCTGGTGGCCCTGGAAAACCAA | |
| | CACACAATCGACCTGACAGACAGCGAAATGAA | |
| | CAAACTGTTCGAGAGAACCAGAAAACAGCTGA | |
| | GAGAGAACGCCGAGGACATGGGCAACGGCTG | |
| | TTTCAAGATCTACCACAAGTGCGACAACGCCT | |
| | GCATTGGCTCCATCAGAAACGGCACCTACGAT | |
| | CACGATGTTTACCGGGACGAGGCCCTGAACAA | |
| | TCGTTTCCAGATCAAGGGAGTGGAGCTGAAGT | |
| | CT | |
| 29 | TATLCLGHHAVPNGTLVKTITNDQIEVTNATELV | A/Memphis/1/1980 |
| | QSSSTGRICDSPHRILDGKNCTLVDALLGDPHCD | mature HA Amino |
| | GFQNEKWDLFVERSKAFSNCYPYDVPDYASLRS | Acid Sequence |
| | LVASSGTLEFINESFNWTGVTQSGGSYACKRGSD | |
| | NSFFSRLNWLYESESKYPVLNVTMPNNGNFDKL | |
| | YIWGVHHPSTDKEQTNLYVRASGRVTVSTKRSQ | |
| | QTIIPNIGSRPWVRGLSSRISIYWTIVKPGDILLINS | |
| | NGNLIAPRGYFKIRTGKSSIMRSDAPIGTCSSECIT | |
| | PNGSIPNDKPFQNVNKITYGACPKYVKQNTLKLA | |
| | TGMRNVPEKQTRGIFGAIAGFIENGWEGMVDGW | |
| | YGFRHQNSEGTGQAADLKSTQAAIDQINGKLNR | |
| | VIEKTNEKFHQIEKEFSEVEGRIQDLEKYVEDTKI | |
| | DLWSYNAELLVALENQHTIDLTDSEMNKLFEKT | |
| | RRQLRENAEDMGNGCFKIYHKCDNACIGSIRNG | |
| | TYDHDVYRDEALNNRFQIKGVELKS | |
| 30 | ACCGCCACCCTGTGCCTGGGCCACCACGCTGT | A/Memphis/1/1980 |
| | GCCAAACGGCACCCTGGTGAAGACCATCACCA | mature HA nucleic acid |
| | ACGATCAGATTGAAGTGACCAATGCCACAGAG | sequence |
| | CTGGTGCAGAGCTCCTCCACTGGCAGAATCTG | |
| | CGACTCCCCTCACAGAATCCTGGATGGCAAAA | |
| | ACTGCACACTCGTGGATGCCCTGCTGGGCGAC | |
| | CCCCACTGCGACGGCTTCCAGAATGAGAAGTG | |
| | GGACCTGTTCGTGGAGAGAAGCAAGGCCTTCA | |
| | GCAACTGCTACCCTTACGACGTCCCTGACTAC | |
| | GCCAGCCTGAGAAGCCTGGTGGCCTCCAGCGG | |
| | CACACTGGAGTTCATCAATGAGTCCTTCAACT | |
| | GGACAGGCGTGACACAGTCTGGCGGCTCTTAC | |
| | GCCTGCAAGCGGGGCAGCGACAACAGCTTCTT | |
| | CTCCCGGCTGAATTGGCTGTACGAGAGCGAGA | |
| | GCAAGTACCCTGTGCTGAACGTGACCATGCCA | |
| | AACAATGGCAATTTCGACAAACTGTACATCTG | |
| | GGGAGTGCACCACCCCAGCACCGACAAGGAG | |
| | CAGACCAACCTCTACGTGAGAGCCAGCGGCAG | |
| | AGTGACCGTGTCCACAAAGCGGAGCCAGCAGA | |
| | CCATCATCCCCAACATCGGCAGCCGGCCCTGG | |
| | GTGAGAGGCCTGAGCTCCCGGATCAGCATCTA | |
| | CTGGACAATCGTGAAACCCGGAGACATCCTGC | |
| | TGATCAACAGCAACGGCAACCTGATCGCCCCC | |
| | AGAGGCTACTTTAAGATCAGAACAGGCAAGAG | |
| | CTCCATCATGAGAAGCGATGCCCCCATCGGCA | |
| | CATGCAGCAGCGAGTGTATCACCCCCAACGGC | |
| | TCAATCCCCAACGACAAGCCTTTCCAGAACGT | |
| | GAACAAGATTACCTATGGCGCCTGCCCCAAGT | |
| | ACGTGAAGCAGAACACACTGAAGCTGGCCACA | |
| | GGGATGAGGAATGTCCCCGAAAAGCAGACAA | |
| | GAGGCATCTTTGGGGCCATCGCCGGCTTCATC | |
| | GAGAACGGCTGGGAAGGAATGGTGGACGGCT | |
| | GGTACGGCTTCAGGCACCAGAACAGCGAGGGC | |
| | ACCGGCCAGGCCGCTGACCTGAAGTCCACCCA | |
| | GGCCGCCATCGACCAGATCAATGGAAAGCTGA | |
| | ACAGAGTGATCGAGAAGACAAACGAAAAGTT | |
| | CCACCAGATCGAAAAGGAATTCTCCGAGGTGG | |
| | AAGGACGGATTCAGGACCTGGAAAAGTACGTG | |
| | GAAGACACCAAGATCGACCTGTGGAGCTACAA | |
| | CGCAGAGCTGCTGGTGGCTCTGGAGAACCAGC | |
| | ACACAATCGACCTGACAGACAGCGAGATGAAC | |
| | AAACTGTTCGAAAAGACACGGAGACAGCTGA | |
| | GGGAGAACGCTGAAGACATGGGAAACGGCTG | |
| | CTTTAAGATTTACCACAAGTGCGACAATGCCT | |
| | GCATTGGCAGCATTCGGAACGGCACATACGAC | |
| | CATGATGTCTACAGAGACGAGGCTCTGAACAA | |
| | CCGGTTCCAGATCAAGGGCGTGGAGCTGAAGT | |
| | CT | |
| 31 | TATLCLGHHAVPNGTLVKTITNDQIEVTNATELV | A/Bilthoven/1761/1976 |
| | QSSSTGKICDNPHRILDGINCTLIDALLGDPHCDG | mature HA Amino |
| | FQNEKWDLFVERSKAFSNCYPYDVPDYASLRSL | Acid Sequence |
| | VASSGTLEFINEGFNWTGVTQNGGSSACKRGPD | |
| | NGFFSRLNWLYKSGSTYPVQNVTMPNNDNSDKL | |
| | YIWGVHHPSTDKEQTDLYVQASGKVTVSTKRSQ | |
| | QTVIPNVGSRPWVRGLSSRVSIYWTIVKPGDILVI | |
| | NSNGNLIAPRGYFKMRTGKSSIMRSDAPIGTCSSE | |
| | CITPNGSIPNDKPFQNVNKITYGACPKYVKQNTL | |
| | KLATGMRNVPEKQTRGIFGAIAGFIENGWEGMID | |
| | GWYGFRHQNSEGTGQAADLKSTQAAIDQINGKL | |
| | NRVIEKTNEKFHQIEKEFSEVEGRIQDLEKYVEDT | |
| | KIDLWSYNAELLVALENQHTIDLTDSEMNKLFE | |
| | KTRRQLRENAEDMGNGCFKIYHKCDNACIGSIR | |
| | NGTYDHDVYRDEALNNRFQIKGVELKS | |
| 32 | ACCGCCACCCTGTGCCTCGGGCACCACGCTGT | A/Bilthoven/1761/1976 |
| | GCCAAACGGCACCCTGGTGAAGACCATCACCA | mature HA nucleic acid |
| | ACGATCAGATCGAAGTGACCAATGCCACCGAA | sequence |
| | CTGGTGCAGAGCTCCTCCACCGGCAAGATCTG | |
| | CGACAACCCTCACAGAATCCTGGACGGCATCA | |
| | ACTGCACCCTGATCGATGCCCTGCTGGGCGAC | |
| | CCTCACTGCGATGGCTTCCAGAATGAGAAGTG | |
| | GGACCTGTTCGTCGAGAGAAGCAAGGCCTTTT | |
| | CCAACTGTTACCCTTACGACGTCCCCGATTACG | |
| | CCAGCCTGAGATCCCTGGTGGCCTCCAGCGGA | |
| | ACACTGGAGTTCATCAACGAGGGCTTCAACTG | |
| | GACAGGCGTGACCCAGAACGGCGGCTCCTCTG | |
| | CCTGCAAGAGAGGCCCCGACAATGGCTTTTTC | |
| | AGCAGACTGAACTGGCTGTACAAGAGCGGCAG | |
| | CACCTACCCAGTCCAGAACGTGACCATGCCAA | |
| | ACAACGACAACTCCGACAAGCTGTACATCTGG | |
| | GGCGTCCACCACCCCAGCACAGACAAGGAGCA | |
| | GACAGACCTCTACGTCCAAGCCTCCGGCAAGG | |
| | TCACCGTGTCCACCAAGCGGAGCCAGCAGACC | |
| | GTGATCCCCAACGTGGGCAGCCGGCCCTGGGT | |
| | GAGAGGCCTGAGCTCCCGGGTGTCCATCTACT | |
| | GGACCATCGTGAAACCAGGCGACATCCTGGTG | |
| | TGCTCCTCTGAATGCATTACACCCAACGGAAG | |
| | ATCAACAGCAACGGCAACCTGATCGCCCCTCG | |
| | GGGCTACTTCAAGATGAGAACCGGCAAGAGCA | |
| | GCATCATGAGAAGCGATGCCCCAATCGGAACA | |
| | CATCCCCAACGACAAACCCTTCCAGAACGTGA | |
| | ACAAGATTACATATGGCGCCTGCCCCAAGTAC | |
| | GTGAAGCAGAACACACTGAAGCTGGCCACAG | |
| | GAATGAGAAACGTCCCTGAGAAGCAGACAAG | |
| | AGGCATCTTTGGCGCTATCGCCGGATTCATCG | |
| | AAAACGGCTGGGAGGGAATGATCGACGGCTG | |
| | GTACGGATTTAGACACCAAAACAGCGAGGGCA | |
| | CCGGCCAGGCCGCTGACCTGAAGTCAACCCAG | |
| | GCCGCCATCGACCAGATCAACGGCAAGCTGAA | |
| | CAGAGTGATCGAAAAGACCAATGAGAAGTTCC | |
| | ATCAGATCGAGAAGGAGTTCAGCGAAGTGGA | |
| | GGGCAGAATCCAGGACCTGGAGAAGTACGTG | |
| | GAGGATACCAAGATCGACCTGTGGAGCTACAA | |
| | CGCAGAGCTGCTGGTGGCCCTGGAGAACCAGC | |
| | ACACCATCGACCTGACAGACAGCGAGATGAAC | |
| | AAGCTGTTCGAGAAGACAAGAAGACAGCTGA | |
| | GAGAGAACGCCGAGGATATGGGCAACGGCTG | |
| | TTTCAAGATCTATCACAAGTGCGACAACGCCT | |
| | GCATTGGCAGCATCAGAAACGGCACCTACGAC | |
| | CACGATGTCTACAGAGACGAGGCTCTGAACAA | |
| | CAGATTCCAGATCAAGGGCGTGGAGCTGAAGT | |
| | CC | |
| 33 | TATLCLGHHAVPNGTLVKTITDDQIEVTNATELV | A/Hong_Kong/1/1968 |
| | QSSSTGKICNNPHRILDGIDCTLIDALLGDPHCDV | mature HA Amino |
| | FQNETWDLFVERSKAFSNCYPYDVPDYASLRSL | Acid Sequence |
| | VASSGTLEFITEGFTWTGVTQNGGSNACKRGPGS | |
| | GFFSRLNWLTKSGSTYPVLNVTMPNNDNFDKLY | |
| | IWGVHHPSTNQEQTSLYVQASGRVTVSTRRSQQ | |
| | TIIPNIGSRPWVRGLSSRISIYWTIVKPGDVLVINS | |
| | NGNLIAPRGYFKMRTGKSSIMRSDAPIDTCISECI | |
| | TPNGSIPNDKPFQNVNKITYGACPKYVKQNTLKL | |
| | ATGMRNVPEKQTRGLFGAIAGFIENGWEGMIDG | |
| | WYGFRHQNSEGTGQAADLKSTQAAIDQINGKLN | |
| | RVIEKTNEKFHQIEKEFSEVEGRIQDLEKYVEDTK | |
| | IDLWSYNAELLVALENQHTIDLTDSEMNKLFEKT | |
| | RRQLRENAEDMGNGCFKIYHKCDNACIESIRNGT | |
| | YDHDVYRDEALNNRFQIKGVELKS | |
| 34 | ACAGCCACCCTGTGCCTGGGCCACCACGCTGT | A/Hong_Kong/1/1968 |
| | GCCAAACGGCACACTGGTGAAGACAATCACCG | mature HA nucleic acid |
| | ACGACCAGATCGAGGTGACCAATGCAACAGA | sequence |
| | GCTGGTGCAGAGTTCCAGCACCGGAAAGATCT | |
| | GCAACAACCCTCACCGGATCCTGGACGGCATC | |
| | GACTGCACCCTGATTGATGCCCTGCTCGGCGA | |
| | CCCTCACTGCGACGTGTTCCAGAACGAAACAT | |
| | GGGACCTGTTCGTGGAGCGGTCCAAGGCCTTT | |
| | AGCAACTGCTACCCCTACGACGTCCCCGATTA | |
| | CGCCAGCCTGAGATCTCTCGTGGCCTCCAGCG | |
| | GCACCCTGGAGTTCATTACAGAGGGCTTCACA | |
| | TGGACAGGCGTGACACAGAACGGCGGCAGCA | |
| | ACGCCTGCAAGAGAGGCCCTGGCTCTGGCTTC | |
| | TTCAGCAGGCTGAATTGGCTGACAAAGAGCGG | |
| | AAGCACCTACCCTGTGCTGAACGTGACCATGC | |
| | CAAACAACGACAATTTCGACAAGCTGTACATC | |
| | TGGGGCGTCCACCACCCCAGCACCAACCAGGA | |
| | GCAGACCAGCCTCTATGTCCAGGCCAGCGGCA | |
| | GAGTGACAGTGAGCACCAGACGGTCCCAGCAG | |
| | ACCATCATCCCTAACATCGGATCCAGACCTTG | |
| | GGTGCGGGGCCTGAGCAGCAGAATCTCCATCT | |
| | ACTGGACCATCGTGAAACCTGGCGATGTCCTG | |
| | GTGATCAACTCCAACGGCAACCTGATCGCCCC | |
| | TCGGGGCTACTTCAAGATGCGGACAGGCAAGA | |
| | GCTCAATCATGAGAAGCGATGCTCCTATCGAC | |
| | ACCTGCATCAGCGAGTGTATCACACCCAACGG | |
| | CTCTATCCCCAACGACAAGCCCTTCCAGAACG | |
| | TGAACAAGATCACATATGGAGCCTGCCCCAAG | |
| | TACGTGAAACAGAACACCCTGAAGCTGGCCAC | |
| | CGGGATGAGAAACGTCCCTGAGAAGCAGACCC | |
| | GGGGCCTGTTTGGAGCCATCGCCGGCTTCATC | |
| | GAGAACGGCTGGGAAGGCATGATCGACGGCT | |
| | GGTACGGATTCCGGCACCAGAACAGCGAAGGC | |
| | ACCGGCCAGGCTGCTGACCTGAAGTCCACACA | |
| | GGCCGCCATCGACCAGATCAATGGCAAGCTGA | |
| | ACAGAGTGATCGAAAAGACAAACGAAAAGTT | |
| | CCACCAGATCGAAAAGGAGTTCAGCGAAGTGG | |
| | AGGGCAGAATCCAGGACCTGGAGAAGTACGT | |
| | GGAGGACACCAAGATCGACCTGTGGAGCTACA | |
| | ACGCAGAACTGCTGGTCGCCCTGGAGAACCAG | |
| | CACACCATCGACCTGACAGATTCGGAGATGAA | |
| | CAAGCTGTTCGAGAAAACCCGGAGACAGCTGA | |
| | GAGAGAACGCCGAGGACATGGGAAACGGCTG | |
| | CTTCAAGATTTACCACAAGTGCGACAACGCCT | |
| | GCATTGAATCCATTCGGAACGGCACCTACGAC | |
| | CACGATGTCTACAGAGACGAGGCCCTGAACAA | |
| | CCGGTTTCAGATCAAGGGCGTGGAGCTGAAGT | |
| | CC | |
| 35 | ATLCLGHHAVPNGTLVKTITDDQIEVTNATELVQ | A/Indiana/08/2011 |
| | SSSTGGICNSPHQILDGKNCTLIDALLGDPHCDDF | mature HA Amino |
| | QNKEWDLFVERSTAYSNCYPYYVPDYATLRSLV | Acid Sequence |
| | ASSGNLEFTQESFNWTGVAQGGSSYACRRGSVN | |
| | SFFSRLNWLYNLNYKYPEQNVTMPNNDKFDKL | |
| | YIWGVHHPGTDKDQTNLYVQASGRVIVSTKRSQ | |
| | QTVIPNIGSRPWVRGVSSIISIYWTIVKPGDILLINS | |
| | TGNLIAPRGYFKIQSGKSSIMRSDAHIDECNSECIT | |
| | PNGSIPNDKPFQNVNKITYGACPRYVKQNTLKLA | |
| | TGMRNVPEKQTRGIFGAIAGFIENGWEGMVDGW | |
| | YGFRHQNSEGTGQAADLKSTQAAINQITGKLNR | |
| | VIKKTNEKFHQIEKEFSEVEGRIQDLEKYVEDTKI | |
| | DLWSYNAEILVALENQHTIDLTDSEMSKLFERTR | |
| | RQLRENAEDMGNGCFKIYHKCDNACIGSIRNGT | |
| | YDHDIYRNEALNNRFQIKGVQLKS | |
| 36 | GCCACCCTGTGTCTCGGGCACCACGCAGTCCC | A/Indiana/08/2011 |
| | CAACGGCACCCTGGTGAAGACAATCACAGATG | mature HA nucleic acid |
| | ACCAGATCGAAGTGACCAATGCCACAGAGCTG | sequence |
| | GTGCAGAGCAGCTCCACCGGAGGCATCTGCAA | |
| | TTCACCCCACCAGATCCTGGACGGCAAGAACT | |
| | GCACCCTGATCGATGCCCTGCTGGGCGACCCT | |
| | CACTGCGACGACTTTCAGAACAAGGAGTGGGA | |
| | CCTGTTCGTGGAGAGAAGCACCGCCTACAGCA | |
| | ACTGCTACCCTTACTACGTCCCTGACTACGCCA | |
| | CACTGAGGAGCCTGGTGGCCTCCAGCGGCAAC | |
| | CTGGAATTCACCCAGGAGAGCTTCAATTGGAC | |
| | AGGAGTGGCCCAGGGCGGCAGCTCCTATGCCT | |
| | GCCGGAGAGGCTCCGTGAACAGCTTCTTCAGC | |
| | AGACTGAACTGGCTGTACAATCTGAACTACAA | |
| | GTACCCCGAACAGAACGTGACCATGCCAAACA | |
| | ACGACAAGTTCGACAAGCTGTACATCTGGGGA | |
| | GTGCACCACCCCGGCACCGACAAGGATCAGAC | |
| | AAACCTCTACGTCCAGGCTTCTGGCCGGGTGA | |
| | TCGTGTCCACCAAGAGAAGCCAGCAGACAGTG | |
| | ATCCCCAACATCGGCAGCCGGCCTTGGGTCCG | |
| | GGGCGTGTCCAGCATCATCAGCATCTACTGGA | |
| | CAATCGTGAAGCCAGGCGACATCCTGCTGATC | |
| | AATTCCACCGGCAACCTGATCGCCCCCAGAGG | |
| | CTACTTCAAGATCCAGAGCGGCAAAAGCAGCA | |
| | TCATGAGAAGCGATGCCCACATTGATGAATGC | |
| | AACTCTGAGTGTATCACCCCCAACGGCAGCAT | |
| | CCCCAATGACAAGCCCTTCCAGAACGTGAACA | |
| | AGATCACCTATGGCGCCTGCCCCAGGTACGTG | |
| | AAGCAGAACACACTGAAGCTGGCCACAGGAA | |
| | TGAGAAATGTCCCCGAGAAGCAGACCCGGGGC | |
| | ATCTTTGGAGCCATCGCCGGCTTCATCGAAAA | |
| | CGGCTGGGAAGGAATGGTGGACGGCTGGTACG | |
| | GCTTCCGGCACCAGAACAGCGAGGGCACAGGC | |
| | CAGGCCGCTGACCTGAAGTCAACACAGGCCGC | |
| | CATCAATCAGATCACAGGCAAGCTGAACAGAG | |
| | TGATCAAGAAGACCAATGAGAAGTTCCACCAG | |
| | ATCGAGAAGGAGTTCTCCGAAGTGGAGGGCCG | |
| | GATCCAGGACCTGGAAAAGTACGTGGAGGAC | |
| | ACAAAGATTGACCTGTGGTCTTACAACGCCGA | |
| | GATCCTGGTGGCCCTGGAAAACCAGCACACCA | |
| | TCGACCTGACCGACAGCGAGATGTCCAAGCTG | |
| | TTTGAGAGAACACGGCGGCAGCTGAGAGAAA | |
| | ACGCCGAGGACATGGGCAACGGCTGCTTCAAG | |
| | ATTTACCACAAGTGCGACAACGCCTGCATTGG | |
| | CAGCATCAGAAACGGCACATACGACCACGACA | |
| | TTTACAGAAATGAGGCCCTGAACAACAGATTT | |
| | CAGATCAAGGGAGTGCAGCTGAAGTCT | |
| 37 | TDNLWVTVYYGVPVWKDADTTLFCASDAKAHE | Centi-HIV-01 Full |
| | TEVHNIWATHACVPTDPNPQEIPLENVTENFNM | Sequence |
| | WKNNMVEQMQEDVISLWDQSLKPCVKLTPLCV | |
| | TLNCTNANLTNVNISISVSNVSIGNITDEVSNCSF | |
| | DVTTEITDRKQRVHALFYKLDLVPMNKDESSEYI | |
| | LIHCNTSVIKQACPKISFDPIPIHYCTPAGYAILKC | |
| | NDKNFNGTGPCKNVSSVQCTHGIKPVVSTQLLL | |
| | NGSLAEEEIIIRSKNLTDNAKTIIVHLNKSVVIDCT | |
| | RPSNNTRTGMTIGPGRVFYKTGEIVGDIRKAYCQ | |
| | INRTNWFNALKQVAEKLKGHFNNKTIIFQPPSGG | |
| | DPEITMHHFNCRGEFFYCNTSQLFNFTYNGTQTT | |
| | EGKDNITLPCRIKQVIKMWQRVGQAMYAPPISGI | |
| | INCVSNITGILLTRDGGDNDSTNETFRPGGGDMR | |
| | DNWRSELYKYKVVQIEPLGIAPTKCKRRVVEggs | |
| | ggggsggggsggggsggAVGIGALIFGFLGAAGSTMGA | |
| | ASITLTVQARQLLSGIVQQQSNLLRAIEAQQHML | |
| | QLTVWGIKQLQARVLAVERYLKDQQFLGLWGC | |
| | SGKIICCTAVPWNSTWSNKSLEEIWNNMTWIEW | |
| | EREISNYTSKIYEILTESQNQQDRNEKDLLELDGS | |
| | LEVLFQGPGSGYIPEAPRDGQAYVRKDGEWVLL | |
| | STFLHHHHHHHHHH | |
| 38 | TDNLWVTVYYGVPVWKDADTTLFCASDAKAHE | Centi-HIV-01 gp120 |
| | TEVHNIWATHACVPTDPNPQEIPLENVTENFNM | |
| | WKNNMVEQMQEDVISLWDQSLKPCVKLTPLCV | |
| | TLNCTNANLTNVNISISVSNVSIGNITDEVSNCSF | |
| | DVTTEITDRKQRVHALFYKLDLVPMNKDESSEYI | |
| | LIHCNTSVIKQACPKISFDPIPIHYCTPAGYAILKC | |
| | NDKNFNGTGPCKNVSSVQCTHGIKPVVSTQLLL | |
| | NGSLAEEEIIIRSKNLTDNAKTIIVHLNKSVVIDCT | |
| | RPSNNTRTGMTIGPGRVFYKTGEIVGDIRKAYCQ | |
| | INRTNWFNALKQVAEKLKGHFNNKTIIFQPPSGG | |
| | DPEITMHHFNCRGEFFYCNTSQLFNFTYNGTQTT | |
| | EGKDNITLPCRIKQVIKMWQRVGQAMYAPPISGI | |
| | INCVSNITGILLTRDGGDNDSTNETFRPGGGDMR | |
| | DNWRSELYKYKVVQIEPLGIAPTKCKRRVVE | |
| 39 | ggsggggsggggsggggsgg | Centi-HIV-01 linker |
| 40 | AVGIGALIFGFLGAAGSTMGAASITLTVQARQLL | Centi-HIV-01 gp41 |
| | SGIVQQQSNLLRAIEAQQHMLQLTVWGIKQLQA | |
| | RVLAVERYLKDQQFLGLWGCSGKIICCTAVPWN | |
| | STWSNKSLEEIWNNMTWIEWEREISNYTSKIYEIL | |
| | TESQNQQDRNEKDLLELD | |
| 41 | AQNLWVTVYYGVPVWRDADTTLFCASDAKAY | Centi-HIV-03 Full |
| | KTEVHNVWATHACVPTDPNPQEIYLTNVTENFN | Sequence |
| | MWKNKMVEQMHEDIISLWDQSLKPCVQLTPLC | |
| | VTLNCSDVTNNTLRNATVNANANATVTGDMEG | |
| | EMKNCSYNMTTAVRDKQKKVYSLFYRLDVVQI | |
| | SNNSSSSSEYRLINCNTSAITQACPKVTFEPIPIHY | |
| | CAPAGFAILKCNEEGFNGTGPCKNVSTVQCTHGI | |
| | KPVVSTQLLLNGSLAEKGVIIRSENISNNAKTIIVQ | |
| | LAEPVTINCTRPNNNTRKGIHIGPGRAFYATGDII | |
| | GDIRKAYCNVSRTQWNKTLAQVAAQLTKYWNK | |
| | TINFTSSSGGDVEITTHSFNCGGEFFYCNTTNLFN | |
| | STWRGNKTVSNSTELDENGTITLPCRIKQIINMW | |
| | QRTGQAMYAPPIQGVIKCVSNITGLLLTRDGGSD | |
| | NNISSETFRPGGGDMRDNWRSELYKYKVVKLEP | |
| | LGVAPNKCRRRVVEggsggggsggggsggggsggAVGL | |
| | GAVFIGFLGAAGSTMGAASITLTAQARQLLSGIV | |
| | QQQSNLLKAIEAQQHLLKLTVWGIKQLQARVLA | |
| | LERYLKDQQLLGIWGCSGKLICCTSVPWNSSWS | |
| | NKSFEQIWNNMTWLEWDKEVSNYTQIIYELLEV | |
| | SQNQQEKNEQDLLSLDGSLEVLFQGPGSGYIPEA | |
| | PRDGQAYVRKDGEWVLLSTFLHHHHHHHHHH | |
| 42 | AQNLWVTVYYGVPVWRDADTTLFCASDAKAY | Centi-HIV-03 gp120 |
| | KTEVHNVWATHACVPTDPNPQEIYLTNVTENFN | |
| | MWKNKMVEQMHEDIISLWDQSLKPCVQLTPLC | |
| | VTLNCSDVTNNTLRNATVNANANATVTGDMEG | |
| | EMKNCSYNMTTAVRDKQKKVYSLFYRLDVVQI | |
| | SNNSSSSSEYRLINCNTSAITQACPKVTFEPIPIHY | |
| | CAPAGFAILKCNEEGFNGTGPCKNVSTVQCTHGI | |
| | KPVVSTQLLLNGSLAEKGVIIRSENISNNAKTIIVQ | |
| | LAEPVTINCTRPNNNTRKGIHIGPGRAFYATGDII | |
| | GDIRKAYCNVSRTQWNKTLAQVAAQLTKYWNK | |
| | TINFTSSSGGDVEITTHSFNCGGEFFYCNTTNLFN | |
| | STWRGNKTVSNSTELDENGTITLPCRIKQIINMW | |
| | QRTGQAMYAPPIQGVIKCVSNITGLLLTRDGGSD | |
| | NNISSETFRPGGGDMRDNWRSELYKYKVVKLEP | |
| | LGVAPNKCRRRVVE | |
| 43 | ggsggggsggggsggggsgg | Centi-HIV-03 linker |
| 44 | AVGLGAVFIGFLGAAGSTMGAASITLTAQARQL | Centi-HIV-03 gp41 |
| | LSGIVQQQSNLLKAIEAQQHLLKLTVWGIKQLQA | |
| | RVLALERYLKDQQLLGIWGCSGKLICCTSVPWN | |
| | SSWSNKSFEQIWNNMTWLEWDKEVSNYTQIIYE | |
| | LLEVSQNQQEKNEQDLLSLD | |
| 45 | ADNLWVTVYYGVPVWKEATTTLFCASDAKAYK | Centi-HIV-04 Full |
| | EEAHNIWATHACVPTDPNPQEVKLENVTENFNM | Sequence |
| | WKNNMVDQMHEDIISLWDQSLKPCVKLTPLCVT | |
| | LNCIEWKNNSTNVTDDGNNSTNAPDDIGVKNCS | |
| | FNITTEVRDKKKQVYALFYKLDVVQIDDSSNTSY | |
| | RLINCNTSAITQACPKVSFEPIPIHYCAPAGFAILK | |
| | CNDKTFNGTGPCTNVSTVQCTHGIKPVVSTQLLL | |
| | NGSLAEGEIMIRSENLTNNAKTIIVQFNQSVEISCT | |
| | RPNNNTRKSVRIGPGQAFYATGEIIGDIRQAYCNI | |
| | NKAKWNKTLQQVAKKLRDLLNKTTINFKPHSG | |
| | GDLEITTHSFNCGGEFFYCNTSKLENNSIGRNATT | |
| | SNSTESITLPCKIKQIINMWQGVGKAMYAPPIEGL | |
| | IKCESNITGLLLTRDGGNTNNQNETFRPGGGDMR | |
| | DNWRSELYKYKVVRIEPLGLAPTRCKRRVVEggs | |
| | ggggsggggsggggsggAIGLGALFLGFLGAAGSTMGA | |
| | ASLTLTVQARQVLSGIVQQQNNLLRAIEAQQHLL | |
| | QLTVWGIKQLQARILAVERYLRDQQLLGIWGCS | |
| | GKHICCTTVPWNSSWSNKSLEYIWQNMTWMEW | |
| | EKEIDNYTGLIYSLIEESQTQQEKNEKELLELDGS | |
| | LEVLFQGPGSGYIPEAPRDGQAYVRKDGEWVLL | |
| | STFLHHHHHHHHHH | |
| 46 | ADNLWVTVYYGVPVWKEATTTLFCASDAKAYK | Centi-HIV-04 gp120 |
| | EEAHNIWATHACVPTDPNPQEVKLENVTENFNM | |
| | WKNNMVDQMHEDIISLWDQSLKPCVKLTPLCVT | |
| | LNCIEWKNNSTNVTDDGNNSTNAPDDIGVKNCS | |
| | FNITTEVRDKKKQVYALFYKLDVVQIDDSSNTSY | |
| | RLINCNTSAITQACPKVSFEPIPIHYCAPAGFAILK | |
| | CNDKTFNGTGPCTNVSTVQCTHGIKPVVSTQLLL | |
| | NGSLAEGEIMIRSENLTNNAKTIIVQFNQSVEISCT | |
| | RPNNNTRKSVRIGPGQAFYATGEIIGDIRQAYCNI | |
| | NKAKWNKTLQQVAKKLRDLLNKTTINFKPHSG | |
| | GDLEITTHSFNCGGEFFYCNTSKLENNSIGRNATT | |
| | SNSTESITLPCKIKQIINMWQGVGKAMYAPPIEGL | |
| | IKCESNITGLLLTRDGGNTNNQNETFRPGGGDMR | |
| | DNWRSELYKYKVVRIEPLGLAPTRCKRRVVE | |
| 47 | ggsggggsggggsggggsgg | Centi-HIV-04 linker |
| 48 | AIGLGALFLGFLGAAGSTMGAASLTLTVQARQV | Centi-HIV-04 gp41 |
| | LSGIVQQQNNLLRAIEAQQHLLQLTVWGIKQLQ | |
| | ARILAVERYLRDQQLLGIWGCSGKHICCTTVPW | |
| | NSSWSNKSLEYIWQNMTWMEWEKEIDNYTGLI | |
| | YSLIEESQTQQEKNEKELLELD | |
| 49 | KGNLWVTVYYGVPVWKEANPILFCASDAKAYE | Centi-HIV-06 Full |
| | TEVHNVWATHACVPTDPDPQEMVLGNVTENFN | Sequence |
| | MWKNNMVEQMHEDIISLWDQSLKPCVKLTPLC | |
| | VTLSCTAANTTDNSNSTQSTNSSWERIENGEIQN | |
| | CSFNITTNIRDKIQKQYALFNRLDLVPIDRTDNTS | |
| | YRIISCNTSVITQACPKVTFEPIPIHYCAPAGFAILK | |
| | CNEKKFNGTGPCTNVSTVQCTHGIRPVVSTQLLL | |
| | NGSLAEGEIVIRSANLSNNAKTIIVHLNESVDMTC | |
| | IRPNNNTRKSIHIGPGRAFYATDIIGDIRKAHCNIS | |
| | AAKWNTTLYRIAKKLREHFPNKTINFNKSSGGDP | |
| | EIIMHSFNCGGEFFYCNTTQLFNSSWTDGQELNN | |
| | TGQELNSTITLPCRIKQIINRWQEVGKAMYAPPIS | |
| | GPIKCTSNITGLLLTRDGGNSNSSGKNDTETFRPG | |
| | GGDMRDNWRSELYKYKVVKIEPLGVAPTKCRR | |
| | RVVQggsggggsggggggggsggAVGIGAVFLGFLGAA | |
| | GSTMGAASLTLTVQARQLLSGIVQQQNNLLRAIE | |
| | AQQHLLQLTVWGIKQLQARVLAVERYLRDQQL | |
| | LGIWGCSGKLICCTNVPWNASWSNKSLDAIWDN | |
| | MTWMQWEKEIDNYTDFIYRLLEESQNQQEKNE | |
| | QDLLELDGSLEVLFQGPGSGYIPEAPRDGQAYVR | |
| | KDGEWVLLSTFLHHHHHHHHHH | |
| 50 | KGNLWVTVYYGVPVWKEANPILFCASDAKAYE | Centi-HIV-06 gp120 |
| | TEVHNVWATHACVPTDPDPQEMVLGNVTENFN | |
| | MWKNNMVEQMHEDIISLWDQSLKPCVKLTPLC | |
| | VTLSCTAANTTDNSNSTQSTNSSWERIENGEIQN | |
| | CSFNITTNIRDKIQKQYALFNRLDLVPIDRTDNTS | |
| | YRIISCNTSVITQACPKVTFEPIPIHYCAPAGFAILK | |
| | CNEKKFNGTGPCTNVSTVQCTHGIRPVVSTQLLL | |
| | NGSLAEGEIVIRSANLSNNAKTIIVHLNESVDMTC | |
| | IRPNNNTRKSIHIGPGRAFYATDIIGDIRKAHCNIS | |
| | AAKWNTTLYRIAKKLREHFPNKTINFNKSSGGDP | |
| | EIIMHSFNCGGEFFYCNTTQLFNSSWTDGQELNN | |
| | TGQELNSTITLPCRIKQIINRWQEVGKAMYAPPIS | |
| | GPIKCTSNITGLLLTRDGGNSNSSGKNDTETFRPG | |
| | GGDMRDNWRSELYKYKVVKIEPLGVAPTKCRR | |
| | RVVQ | |
| 51 | ggsggggsggggsggggsgg | Centi-HIV-06 linker |
| 52 | AVGIGAVFLGFLGAAGSTMGAASLTLTVQARQL | Centi-HIV-06 gp41 |
| | LSGIVQQQNNLLRAIEAQQHLLQLTVWGIKQLQ | |
| | ARVLAVERYLRDQQLLGIWGCSGKLICCTNVPW | |
| | NASWSNKSLDAIWDNMTWMQWEKEIDNYTDFI | |
| | YRLLEESQNQQEKNEQDLLELD | |
| 53 | VGNLWVTVYYGVPVWKEAKTTLFCASDAKAYE | Centi-HIV-07 Full |
| | KEVHNVWATHACVPTDPNPQEMVLENVTENFN | Sequence |
| | MWKNDMAEQMHEDVISLWDQSLKPCVKLTPLC | |
| | VTLECRNANATKDNGTKGNEQVKITSNREEIKN | |
| | CSFNTTTEIRDKKQKVYALFYRLDLVPLGEEKRN | |
| | DSNSSSNYILINCNTSAIKQACPKVSFDPIPIHYCA | |
| | PAGYAILKCNNKTFNGTGPCNNVSTVQCTHGIKP | |
| | VVSTQLLLNGSLAEGEIIIRSKNLTDNAKTIIVHLN | |
| | QSVEIKCIRPNNNTRKSIRIGPGQTFYATGDIIGDI | |
| | RQAYCTINRTEWNNTLQGVSKKLAEHFPDKTINF | |
| | TSPPAGGDLEITTHSFNCRGEFFYCNTSALFNSTY | |
| | VNSTLFYRPDGNSSTDIIILCRIKQIINMWQEVGQ | |
| | AMYNPPIAGNITCRSNITGLLLLRDGGTGNTTDN | |
| | KTEIFRPGGGDMRDNWRSELYKYKVVEIKPLGIA | |
| | PTTCKRRVVEggsggggsggggsggggsggAVGIGAVFL | |
| | GFLGAAGSTMGAASITLTVQARQLLSGIVQQQSN | |
| | LLRAIEAQQHLLQLTVWGIKQLQTRVLAIERYLK | |
| | DQQLLGIWGCSGKLICCTAVPWNSSWSNRSQEYI | |
| | WGNMTWMQWDREINNYSDTIYRLLEESQNQQE | |
| | KNEKDLLALDGSLEVLFQGPGSGYIPEAPRDGQA | |
| | YVRKDGEWVLLSTFLHHHHHHHHHH | |
| 54 | VGNLWVTVYYGVPVWKEAKTTLFCASDAKAYE | Centi-HIV-07 gp120 |
| | KEVHNVWATHACVPTDPNPQEMVLENVTENFN | |
| | MWKNDMAEQMHEDVISLWDQSLKPCVKLTPLC | |
| | VTLECRNANATKDNGTKGNEQVKITSNREEIKN | |
| | CSFNTTTEIRDKKQKVYALFYRLDLVPLGEEKRN | |
| | DSNSSSNYILINCNTSAIKQACPKVSFDPIPIHYCA | |
| | PAGYAILKCNNKTFNGTGPCNNVSTVQCTHGIKP | |
| | VVSTQLLLNGSLAEGEIIIRSKNLTDNAKTIIVHLN | |
| | QSVEIKCIRPNNNTRKSIRIGPGQTFYATGDIIGDI | |
| | RQAYCTINRTEWNNTLQGVSKKLAEHFPDKTINF | |
| | TSPPAGGDLEITTHSFNCRGEFFYCNTSALFNSTY | |
| | VNSTLFYRPDGNSSTDIIILCRIKQIINMWQEVGQ | |
| | AMYNPPIAGNITCRSNITGLLLLRDGGTGNTTDN | |
| | KTEIFRPGGGDMRDNWRSELYKYKVVEIKPLGIA | |
| | PTTCKRRVVE | |
| 55 | ggsggggsggggsggggsgg | Centi-HIV-07 linker |
| 56 | AVGIGAVFLGFLGAAGSTMGAASITLTVQARQL | Centi-HIV-07 gp41 |
| | LSGIVQQQSNLLRAIEAQQHLLQLTVWGIKQLQT | |
| | RVLAIERYLKDQQLLGIWGCSGKLICCTAVPWNS | |
| | SWSNRSQEYIWGNMTWMQWDREINNYSDTIYR | |
| | LLEESQNQQEKNEKDLLALD | |
| 57 | LGNLWVTVYYGVPVWKEAKATLFCASDAKVYE | Centi-HIV-08 Full |
| | KEIHNVWATHACVPTDPNPQEMVLANVTENFN | Sequence |
| | MWKNDMVDQMQEDIISLWDQSLKPCVKLTPLC | |
| | VTLNCSEVTNSSITTNSSSTTNSSINANDSMSGEM | |
| | RNCSFNTTTEIRDKKRKEHALFYRLDLVPLEDNN | |
| | TTYRLINCNTSTVTQACPKVTFDPIPIHYCAPAGY | |
| | AILKCNNITFNGTGPCTNVSTVQCTHGIKPVVST | |
| | QLLLNGSLAEGKVIIRSENLTNNAKTIIVQFKEAV | |
| | EIMCTRPGNNTRKSVRIGPGQAFYTTNIIGDIRKA | |
| | HCTISEEKWNNTLKEVSEELRKHFPNKTIEFKPSS | |
| | GGDLEITTHSFNCRGEFFYCNTSNLFNRTYSSNST | |
| | NSASNATSNATITLPCRIKQIINMWQGVGQAIYA | |
| | PPVAGNITCNSSITGLLLTRDGGNINGENNDTET | |
| | FRPGGGDMRDNWRSELYKYKVVEIKPLGIAPTK | |
| | CKRRVVEggsggggsggggsggggsggAVGIGAVFLGFL | |
| | GAAGSTMGAASITLTVQARQLLSGIVQQQSNLLR | |
| | AIEAQQHMLQLTVWGIKQLQARVLAIERYLQDQ | |
| | QLLGMWGCSGKLICCTDVPWNLSWSNNKSVEDI | |
| | WNNMTWMQWDKEINNYTQTIYWLLGESQIQQE | |
| | KNEKDLLELDGSLEVLFQGPGSGYIPEAPRDGQA | |
| | YVRKDGEWVLLSTFLHHHHHHHHHH | |
| 58 | LGNLWVTVYYGVPVWKEAKATLFCASDAKVYE | Centi-HIV-08 gp120 |
| | KEIHNVWATHACVPTDPNPQEMVLANVTENFN | |
| | MWKNDMVDQMQEDIISLWDQSLKPCVKLTPLC | |
| | VTLNCSEVTNSSITTNSSSTTNSSINANDSMSGEM | |
| | RNCSFNTTTEIRDKKRKEHALFYRLDLVPLEDNN | |
| | TTYRLINCNTSTVTQACPKVTFDPIPIHYCAPAGY | |
| | AILKCNNITFNGTGPCTNVSTVQCTHGIKPVVST | |
| | QLLLNGSLAEGKVIIRSENLTNNAKTIIVQFKEAV | |
| | EIMCTRPGNNTRKSVRIGPGQAFYTTNIIGDIRKA | |
| | HCTISEEKWNNTLKEVSEELRKHFPNKTIEFKPSS | |
| | GGDLEITTHSFNCRGEFFYCNTSNLFNRTYSSNST | |
| | NSASNATSNATITLPCRIKQIINMWQGVGQAIYA | |
| | PPVAGNITCNSSITGLLLTRDGGNINGENNDTET | |
| | FRPGGGDMRDNWRSELYKYKVVEIKPLGIAPTK | |
| | CKRRVVE | |
| 59 | ggsggggsggggsggggsgg | Centi-HIV-08 linker |
| 60 | AVGIGAVFLGFLGAAGSTMGAASITLTVQARQL | Centi-HIV-08 gp41 |
| | LSGIVQQQSNLLRAIEAQQHMLQLTVWGIKQLQ | |
| | ARVLAIERYLQDQQLLGMWGCSGKLICCTDVPW | |
| | NLSWSNNKSVEDIWNNMTWMQWDKEINNYTQ | |
| | TIYWLLGESQIQQEKNEKDLLELD | |
| 61 | SENLWATVYYGVPVWEDATTPLFCASDAKAYSP | Centi-HIV-09 Full |
| | EKHNVWATHACVPTDPNPQEISLENITENFNMW | Sequence |
| | RNDMVEQMHEDIISLWDESLKPCVKLTPLCVTL | |
| | DCSNVTRSNIGNSTGNNNTVRNSTVNNNQEMKN | |
| | CSFNITTELRDKTKKEYALFYKLDIVPLNSSNNN | |
| | DSSYRLINCNVSTIKQACPKVSFEPIPIHYCAPAGF | |
| | AILKCRDKNFKGTGACKNVSTVQCTHGIKPVVS | |
| | TQLLLNGSLAEGEIMIRSENITDNTKIIIVQLNKSIE | |
| | INCTRPGNNTRRSIRIGPGQSFYATGDIIGDIRQAH | |
| | CTVNKRNWTEMLQGVRTQLKDLLENKNISFNSS | |
| | TGGDLEITTHSFNCRGEFFYCDTSGLFNTTLLTTN | |
| | STDNETITLSCKIKQIVRMWQRVGQAMYAPPIRG | |
| | NITCISNITGLLLTRDGGQTNSSNETFRPGGGNMR | |
| | DNWRSELYKYKIVKIKPLGVAPTRCRRRVVEggs | |
| | ggggsggggsggggsggAVGLGALLLGFLGTAGSTMG | |
| | AASITLTVQARQLLSGIVQQQSNLLRAIEAQQHL | |
| | LQLTVWGIKQLQARLLAVERYLKDQQLLGIWGC | |
| | SGKLICCTNVPWNTSWSNKSFEEIWDNMTWIQW | |
| | DREVSKYTQEIYNLIEESQNQQERNEQDLLALDG | |
| | SLEVLFQGPGSGYIPEAPRDGQAYVRKDGEWVL | |
| | LSTFLHHHHHHHHHH | |
| 62 | SENLWATVYYGVPVWEDATTPLFCASDAKAYSP | Centi-HIV-09 gp120 |
| | EKHNVWATHACVPTDPNPQEISLENITENFNMW | |
| | RNDMVEQMHEDIISLWDESLKPCVKLTPLCVTL | |
| | DCSNVTRSNIGNSTGNNNTVRNSTVNNNQEMKN | |
| | CSFNITTELRDKTKKEYALFYKLDIVPLNSSNNN | |
| | DSSYRLINCNVSTIKQACPKVSFEPIPIHYCAPAGF | |
| | AILKCRDKNFKGTGACKNVSTVQCTHGIKPVVS | |
| | TQLLLNGSLAEGEIMIRSENITDNTKIIIVQLNKSIE | |
| | INCTRPGNNTRRSIRIGPGQSFYATGDIIGDIRQAH | |
| | CTVNKRNWTEMLQGVRTQLKDLLENKNISFNSS | |
| | TGGDLEITTHSFNCRGEFFYCDTSGLFNTTLLTTN | |
| | STDNETITLSCKIKQIVRMWQRVGQAMYAPPIRG | |
| | NITCISNITGLLLTRDGGQTNSSNETFRPGGGNMR | |
| | DNWRSELYKYKIVKIKPLGVAPTRCRRRVVE | |
| 63 | ggsggggsggggsggggsgg | Centi-HIV-09 linker |
| 64 | AVGLGALLLGFLGTAGSTMGAASITLTVQARQL | Centi-HIV-09 gp41 |
| | LSGIVQQQSNLLRAIEAQQHLLQLTVWGIKQLQA | |
| | RLLAVERYLKDQQLLGIWGCSGKLICCTNVPWN | |
| | TSWSNKSFEEIWDNMTWIQWDREVSKYTQEIYN | |
| | LIEESQNQQERNEQDLLALD | |
| 65 | TDQLWVTVYYGVPVWKEATTTLFCASDAKGYD | Centi-HIV-11 Full |
| | TEVHNVWATHACVPTDPNPQEVVMGNVTENFN | Sequence |
| | MWKNSMVDQMHEDIISLWDQSLQPCVKLTPLC | |
| | VTLKCNDTWGNATQTNNSRVVTTPTTDSKLGKR | |
| | EMTNCSFNITSNIRDRVQEEHALFYKFDVVPIKE | |
| | DDNNNNTRYRLIHCNTSVITQACPKVSFEPIPIHY | |
| | CAPAGFAILKCNDNKFNGSGPCRNVSTVQCTHGI | |
| | RPVVSTQLLLNGSLAEEEIVIRSSNFTDNAKTIIVQ | |
| | LNKSVEITCIRPGNNTRKSIPIGPGRAFFATGDVIG | |
| | DIRKAHCTLNRTDWRNTLKQIATKLRRQFENKTI | |
| | SFQKSSGGDPEIVMHSFNCGGEFFYCNTTQLFDS | |
| | NWTLSDITKGSNTTEGNSTHITLPCRIKQIINMWQ | |
| | EVGKAMYAPPIRGNITCISNITGLLLVRDGGNNN | |
| | ETEIFRPGGGDMRDNWRSELYKYKVIKIEPLGIA | |
| | PTKCKRRVVQggsggggsggggsggggsggAALGALFL | |
| | GFLGAAGSTMGAASLTLTVQARLLLSGIVQQQN | |
| | NLLRAIEAQQHLLQLTVWGIKQLQARVLAVERY | |
| | LRDQQLLGIWGCSGRLVCCTDVKWNTTWSNKS | |
| | LEEIWNNMTWMEWEREIENYTSEIYTLIEKSQNQ | |
| | QEKNEQKLLELDGSLEVLFQGPGSGYIPEAPRDG | |
| | QAYVRKDGEWVLLSTFLHHHHHHHHHH | |
| 66 | TDQLWVTVYYGVPVWKEATTTLFCASDAKGYD | Centi-HIV-11 gp120 |
| | TEVHNVWATHACVPTDPNPQEVVMGNVTENFN | |
| | MWKNSMVDQMHEDIISLWDQSLQPCVKLTPLC | |
| | VTLKCNDTWGNATQTNNSRVVTTPTTDSKLGKR | |
| | EMTNCSFNITSNIRDRVQEEHALFYKFDVVPIKE | |
| | DDNNNNTRYRLIHCNTSVITQACPKVSFEPIPIHY | |
| | CAPAGFAILKCNDNKFNGSGPCRNVSTVQCTHGI | |
| | RPVVSTQLLLNGSLAEEEIVIRSSNFTDNAKTIIVQ | |
| | LNKSVEITCIRPGNNTRKSIPIGPGRAFFATGDVIG | |
| | DIRKAHCTLNRTDWRNTLKQIATKLRRQFENKTI | |
| | SFQKSSGGDPEIVMHSFNCGGEFFYCNTTQLFDS | |
| | NWTLSDITKGSNTTEGNSTHITLPCRIKQIINMWQ | |
| | EVGKAMYAPPIRGNITCISNITGLLLVRDGGNNN | |
| | ETEIFRPGGGDMRDNWRSELYKYKVIKIEPLGIA | |
| | PTKCKRRVVQ | |
| 67 | ggsggggsggggsggggsgg | Centi-HIV-11 linker |
| 68 | AALGALFLGFLGAAGSTMGAASLTLTVQARLLL | Centi-HIV-11 gp41 |
| | SGIVQQQNNLLRAIEAQQHLLQLTVWGIKQLQA | |
| | RVLAVERYLRDQQLLGIWGCSGRLVCCTDVKW | |
| | NTTWSNKSLEEIWNNMTWMEWEREIENYTSEIY | |
| | TLIEKSQNQQEKNEQKLLELD | |
| 69 | EEKLWVTVHYGVPVWKEATTTLFCASDAKAYK | Centi-HIV-13 Full |
| | TEAHNVWATHACVPTDPNPREVPLENVTENFNM | Sequence |
| | WKNNMVEQMHEDIISLWDQSLKPCVKLTPLCVI | |
| | LNCSNLNNSTNITSSDQGNLDMREEIKNCSFNITT | |
| | GIGKKVRKDYAIFNRIDIVPIDDNDSKNNSSNNTS | |
| | YMLRSCDTSVITQACPKVTFEPIPIHYCTPAGYAI | |
| | LKCNDKKFNGTGPCKNVSTVQCTHGIKPVVSTQ | |
| | LLLNGSLAEEEVIIRSENITDNGKNIIVQLNETVKI | |
| | NCTRPNNNTRKSIHMGWGRAFYATGAIIGDIRQA | |
| | HCNLSRAEWNKTLEKIAIKLKERVNKTKIIFNQSS | |
| | GGDSEIEMHSFNCGGEFFYCNTTQLFNSTWNGT | |
| | HLSNSIGNETITLPCRIKQIINRWQEVGKAMYAPP | |
| | ISGQISCSSNITGLILTRDGSNDNSSTNETFRPGGG | |
| | NMKDNWRSELYKYKVVEIEPVGLAPTKCKRRV | |
| | VQggsggggsggggsggggsggAVGTLGAMFLGFLGTA | |
| | GSTMGAASLTLTVQARQLMSGIVQQQNNLLRAI | |
| | EAQQHMLQLTVWGIKQLQARVLAVERYLRDQQ | |
| | LLGIWGCSGKLICCTAVPWNTSWSNKYLSYIWN | |
| | NMTWMQWEREIDNYTNLIYNLLEESQNQQEKN | |
| | EQELLELDGSLEVLFQGPGSGYIPEAPRDGQAYV | |
| | RKDGEWVLLSTFLHHHHHHHHHH | |
| 70 | EEKLWVTVHYGVPVWKEATTTLFCASDAKAYK | Centi-HIV-13 gp120 |
| | TEAHNVWATHACVPTDPNPREVPLENVTENFNM | |
| | WKNNMVEQMHEDIISLWDQSLKPCVKLTPLCVI | |
| | LNCSNLNNSTNITSSDQGNLDMREEIKNCSFNITT | |
| | GIGKKVRKDYAIFNRIDIVPIDDNDSKNNSSNNTS | |
| | YMLRSCDTSVITQACPKVTFEPIPIHYCTPAGYAI | |
| | LKCNDKKFNGTGPCKNVSTVQCTHGIKPVVSTQ | |
| | LLLNGSLAEEEVIIRSENITDNGKNIIVQLNETVKI | |
| | NCTRPNNNTRKSIHMGWGRAFYATGAIIGDIRQA | |
| | HCNLSRAEWNKTLEKIAIKLKERVNKTKIIFNQSS | |
| | GGDSEIEMHSFNCGGEFFYCNTTQLFNSTWNGT | |
| | HLSNSIGNETITLPCRIKQIINRWQEVGKAMYAPP | |
| | ISGQISCSSNITGLILTRDGSNDNSSTNETFRPGGG | |
| | NMKDNWRSELYKYKVVEIEPVGLAPTKCKRRV | |
| | VQ | |
| 71 | ggsggggsggggsggggsgg | Centi-HIV-13 linker |
| 72 | AVGTLGAMFLGFLGTAGSTMGAASLTLTVQAR | Centi-HIV-13 gp41 |
| | QLMSGIVQQQNNLLRAIEAQQHMLQLTVWGIKQ | |
| | LQARVLAVERYLRDQQLLGIWGCSGKLICCTAV | |
| | PWNTSWSNKYLSYIWNNMTWMQWEREIDNYT | |
| | NLIYNLLEESQNQQEKNEQELLELD | |
| 73 | ASQLWVTVYYGVPVWKEATTTLFCASNAKAYD | Centi-HIV-14 Full |
| | PEVHNVWATHACVPTDPSPQEVKLNVTENFNM | Sequence |
| | WKNDMVEQMHEDIISIWDQSLTPCVKLTPLCVT | |
| | LNCTNNITYNNNINTTTSNNNTNNTTNDWEKME | |
| | PGEIKNCSFNITTNVRDKVQKTYALFNSLDVVPI | |
| | DSDNSSYMIISCNTSATTQACPKVSFEPIPIHFCAP | |
| | AGFAILKCNSKTFNGTGPCKNVSIVQCTHGIRPV | |
| | VSTQLLLNGSLAEEDVIIRSENFINNAKTILVQLN | |
| | ESVIINCTRPNNNTRKGIHMGPGRTIYATGNIIGDI | |
| | RKAYCNLNKTDWERALKRIGIKLREQFENKTIAF | |
| | NSSSGGDPEIVMHSFNCRGEFFYCNSTPLFHYTW | |
| | DGTNGTWGNNASKGNITLHCRIKQIVNMWQRV | |
| | GRAMYAPPISGPISCSSNITGLLLTRDGGGINETN | |
| | TTETFRPGGGDMKDNWRSELYKYKVVQIEPIGIA | |
| | PTKCKRRVVQggsggggsggggsggggsggAVGVFGAM | |
| | FLGFLGAAGSTMGAASITLTAQARQLLSGIVQQQ | |
| | SNLLRAIEAQQHMLQLTVWGIKQLQARVLAVER | |
| | YLKDQQLLGIWGCSGKLICCTTVPWNASWSNKS | |
| | VNEIWDNMTWMQWEREIDNYTNQIYNLLEKSQ | |
| | NQQEKNEQELLELDGSLEVLFQGPGSGYIPEAPR | |
| | DGQAYVRKDGEWVLLSTFLHHHHHHHHHH | |
| 74 | ASQLWVTVYYGVPVWKEATTTLFCASNAKAYD | Centi-HIV-14 gp120 |
| | PEVHNVWATHACVPTDPSPQEVKLNVTENFNM | |
| | WKNDMVEQMHEDIISIWDQSLTPCVKLTPLCVT | |
| | LNCTNNITYNNNINTTTSNNNTNNTTNDWEKME | |
| | PGEIKNCSFNITTNVRDKVQKTYALFNSLDVVPI | |
| | DSDNSSYMIISCNTSATTQACPKVSFEPIPIHFCAP | |
| | AGFAILKCNSKTFNGTGPCKNVSIVQCTHGIRPV | |
| | VSTQLLLNGSLAEEDVIIRSENFINNAKTILVQLN | |
| | ESVIINCTRPNNNTRKGIHMGPGRTIYATGNIIGDI | |
| | RKAYCNLNKTDWERALKRIGIKLREQFENKTIAF | |
| | NSSSGGDPEIVMHSFNCRGEFFYCNSTPLFHYTW | |
| | DGTNGTWGNNASKGNITLHCRIKQIVNMWQRV | |
| | GRAMYAPPISGPISCSSNITGLLLTRDGGGINETN | |
| | TTETFRPGGGDMKDNWRSELYKYKVVQIEPIGIA | |
| | PTKCKRRVVQ | |
| 75 | ggsggggsggggsggggsgg | Centi-HIV-14 linker |
| 76 | AVGVFGAMFLGFLGAAGSTMGAASITLTAQARQ | Centi-HIV-14 gp41 |
| | LLSGIVQQQSNLLRAIEAQQHMLQLTVWGIKQL | |
| | QARVLAVERYLKDQQLLGIWGCSGKLICCTTVP | |
| | WNASWSNKSVNEIWDNMTWMQWEREIDNYTN | |
| | QIYNLLEKSQNQQEKNEQELLELD | |
| 77 | MRTLWIMAVLLLGVEGNLYQFEKLIQKIVGRSG | Agkistrodon contortrix |
| | VLWYSAYGCYCGWGGQGRPQDATDRCCFVHD | contortrix PLA2 |
| | CCYNKVTSCNPKLDIYTYSVKNRDVVCGGTNPC | |
| | KKQICECDRAAAICFRDNKDTYDSKKYWKYPKK | |
| | NCKEESEPC | |
| 78 | MRLSLTDNRLFSDRSKSPFPDFSPAEAINGKNAIT | Agkistrodon piscivorus |
| | SYGSYGCNCGWGHRGQPKDATDRCCFVHKCCY | leucostoma PLA2 |
| | KKLTDCNHKTDRYSYSWKNKAIICEEKNPCLKE | |
| | MCECDKAVAICLRENLDTYNKKYKAYFKLKCK | |
| | KPDTC | |
| 79 | MAVLLLGVEGNLFQFEKLIKKMTGKSGMLCYSA | Agkistrodon piscivorus |
| | YGCYCGWGGQGRPKDATDRCCFVHDCCYGKV | piscivorus PLA2 |
| | TGCDPKLDSYTYSVENGDVVCGGNDPCKKEICE | |
| | CDRAAAICFRDNKVTYDNKYWRFPPQNCKEESE | |
| | PC | |
| 80 | MRTLWIVAVWLIGVEGSVIEFGTMIIEETGRSPFP | Bitis arietans PLA2 |
| | FYTSYGCYCGLGGKGKPKDDTDRCCFVHDCCY | |
| | GGMPDCSPKTDIYRYHRENGEIICESGTSCEKRIC | |
| | ECDKAAAVCFRENLKTYKNKYMVYPDSLCKEE | |
| | SEKC | |
| 81 | NLVQFKTLIMKIAGRSVVYKYFYGCYCGWGGIG | Bothrops alternatus |
| | QPRDATDRCCFVHDCCYGKVTNCNPKTATYSYT | PLA2 |
| | EENGALVCGGDDPCKKQVCECDRVAAMCFRDN | |
| | KDTYDNKYWFLPPKNCQEDSEPC | |
| 82 | MRTLWIMAVLLVGVEGSLIEFAKMILEETKRLPF | Bothrops asper PLA2 |
| | PYYTTYGCYCGWGGQGQPKDATDRCCFVHDCC | |
| | YGKLSNCKPKTDRYSYSRKSGVIICGEGTPCEKQI | |
| | CECDKAAAVCFRENLRTYKKRYMAYPDLLCKK | |
| | PAEKC | |
| 83 | MRTLWIMAVLLVGVEGNLWQLGKMILLETGKIP | Bothrops atrox PLA2 |
| | AKSYAAYGCNCGVLGRGKPKDATDRCCYVHKC | |
| | CYKKLTGCDPKKDRYSYSWKDKTIVCGENNSCL | |
| | KELCECDKAVAICLRENLDTYNKKYRYNYLKPF | |
| | CKKAEPC | |
| 84 | DLWQFGQMMNDVMREYVVFNYLYYGCYCGW | Bothrops jararaca |
| | GGIGKPRDATDRCCFVHDCCYGKVTGCNPKTDS | PLA2 |
| | YTYTYSEENGDVVCGGDDLCKKQICECDRVAAT | |
| | CFRDNKDTYDTKYWLYGAKNCQEESEPC | |
| 85 | MACSLGGSSPVLLLCVLVFASANLIQFGHIIEHLT | Crotalus atrox PLA2 |
| | GRHPLIYNGYGCYCGLGGSRQPVDATDWCCQV | |
| | HDCCYQALSRRHCKPKMEKYFYSVRKDTVTCG | |
| | GETECRRQTCECDKAAALCFRHSKFQDQYIGYH | |
| | NRLCEGPTPPCQGVCPCWAPTKGG | |
| 86 | MRALWILAVLLLGVEGSLVEFETLIMKIAGRSGV | Crotalus horridus |
| | WYYSSYGCYCGAGGQGWPQDASDRCCFEHDCC | PLA2 |
| | YAKLTGCDPTTDVYTYRQEDGEIVCGGDDPCGT | |
| | QICECDKAAAICFRDSMDTYDHKYWRFSLENCQ | |
| | GESQPC | |
| 87 | GCPLGKAPSRPRRGILQLAGMIQCTTGRTPLAYIR | Daboia russelii PLA2 |
| | YGCYCGWGGRGWPKDQVXXCCFKHDCCYGRA | |
| | EEHSCAPKTWWYPWECQDGKAKCDDIEDKCQK | |
| | MACECDRSAAKCLAKAPYNMTYLFWPDTQCGE | |
| | KGPTCPDD | |
| 88 | MRTLWILAVCLIGVEGNLFQFARMINGKLGAFS | Daboia russelii limitis |
| | VWNYISYGCYCGWGGQGTPKDATDRCCFVHDC | PLA2 |
| | CYGGVKGCNPKLAIYSYSFQRGNIVCGRNNGCL | |
| | RTICECDRVAANCFHQNKNTYNKRYKFLSSYYC | |
| | RQTSEKC | |
| 89 | MRALWIVAVLLVGVEGSLFELGKMIWQETGKNP | Deinagkistrodon acutus |
| | VKNYGLYGCNCGVGGRGEPLDATDRCCFVHKC | PLA2 |
| | CYKKLTDCDSKKDRYSYKWKNKAIVCGKNQPC | |
| | MQEMCECDKAFAICLRENLDTYNKSFRYHLKPL | |
| | CKKTSEQC | |
| 90 | MRALWIVAVWLIGVEGSVVELGKMIIQETGKSPF | Echis ocellatus PLA2 |
| | PSYTSYGCFCGGGEKGTPKDATDRCCFVHSCCY | |
| | DKLPDCSPKTDRYKYQRENGEIICENSTSCKKRIC | |
| | ECDKAVAVCLRENLQTYNKKYTYYPNFLCKGEP | |
| | EKC | |

### NUMBERED EMBODIMENTS

Embodiment 1. A vaccine composition comprising six or more homologous distinct antigen components, wherein any two of said six or more homologous distinct antigen components share less than 95% sequence identity and
   a. wherein the homologous distinct antigen components comprise proteins, and wherein a concentration/amount of a protein in a dose of said human adult vaccine composition is about 1 nanogram (ng) to about 3 micrograms (μg);
   b. wherein the homologous distinct antigen components comprise a plurality of RNA, and wherein a concentration/amount of an RNA of said plurality of RNA in a dose of said vaccine composition is about 1 ng to about 5 μg per dose; or
   c. wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said vaccine composition is about 1 ng to about 5 μg per dose.
Embodiment 2. The vaccine composition of Embodiment 1, wherein said vaccine composition is for administration in a human subject.
Embodiment 3. The vaccine composition of Embodiment 2, wherein said vaccine composition is for administration in an adult who is 18 years of age or older.
Embodiment 4. The vaccine composition of Embodiment 3, wherein said vaccine composition is for administration in an adult who is 25 years of age or older.
Embodiment 5. The vaccine composition of Embodiment 3, wherein said vaccine composition is for administration in an adult who is 50 years of age or older.
Embodiment 6. The vaccine composition of Embodiment 3, wherein said vaccine composition is for administration in an adult who is 75 years of age or older.
Embodiment 7. The vaccine composition of Embodiment 2, wherein said vaccine composition is for administration in a child between the ages of about 1 day old and about 18 years old.
Embodiment 8. The vaccine composition of Embodiment 2, wherein said vaccine composition is for administration in a child between the ages of about 1 day old and about 5 years old.
Embodiment 9. The vaccine composition of Embodiment 2, wherein said vaccine composition is for administration in a child between the ages of about 5 years old and about 18 years old.
Embodiment 10. The vaccine composition of Embodiment 1, wherein said vaccine composition is for administration in an animal.
Embodiment 11. The vaccine composition of Embodiment 10, wherein the animal is a livestock animal.
Embodiment 12. The vaccine composition of Embodiment 11, wherein the livestock animal is a cow, a bull, an alpaca, a llama, a sheep, a pig, or a bird.
Embodiment 13. The vaccine composition of Embodiment 10, wherein the animal is a domesticated animal.
Embodiment 14. The vaccine composition of Embodiment 13, wherein the domesticated animal is a primate.
Embodiment 15. The vaccine composition of Embodiment 1, wherein six or more homologous distinct antigen components comprise an antigen selected from the group consisting of a virus, a bacterium, a fungus, a prion, a plant, or a combination thereof.
Embodiment 16. The vaccine composition of Embodiment 1, wherein a concentration/amount of a homologous distinct antigen components component of said homologous distinct antigen components in a dose of said vaccine composition is about 1 nanogram (ng) to about 5 micrograms (μg).
Embodiment 17. The vaccine composition of Embodiment 1, wherein a concentration/amount of a homologous distinct antigen components component of said homologous distinct antigen components in a dose of said vaccine composition is about 1 nanogram (ng) to about 1 microgram (μg).
Embodiment 18. The vaccine composition of any one of Embodiment 1-Embodiment 17, wherein the homologous distinct antigen components comprise a component of a virus.
Embodiment 19. The vaccine composition of Embodiment 18, wherein the component of the virus is a receptor-binding domain.
Embodiment 20. The vaccine composition of Embodiment 18, wherein at least two homologous distinct antigen components of said six or more homologous distinct antigen components comprise receptor binding domains for a cell surface protein.
Embodiment 21. The vaccine composition of Embodiment 20, wherein said cell surface protein is mammalian.
Embodiment 22. The vaccine composition of Embodiment 20, wherein said receptor binding domains of said at least two homologous distinct antigen components of said six or more homologous distinct antigen components share less than 99% sequence identity.
Embodiment 23. The vaccine composition of Embodiment 22, wherein said receptor binding domains of said at least two homologous distinct antigen components of said six or more homologous distinct antigen components share less than 98% sequence identity.
Embodiment 24. The vaccine composition of Embodiment 23, wherein said receptor binding domains of said at least two homologous distinct antigen components of said six or more homologous distinct antigen components share less than 95% sequence identity.
Embodiment 25. The vaccine composition of Embodiment 24, wherein said receptor binding domains of said at least two homologous distinct antigen components of said six or more homologous distinct antigen components share less than 95% sequence identity.
Embodiment 26. The vaccine composition of Embodiment 25, wherein said receptor binding domains of said at least two homologous distinct antigen components of said six or more homologous distinct antigen components share less than 90% sequence identity.
Embodiment 27. The vaccine composition of Embodiment 26, wherein said receptor binding domains of said at least two homologous distinct antigen components of said six or more homologous distinct antigen components share less than 85% sequence identity.
Embodiment 28. The vaccine composition of Embodiment 27, wherein said receptor binding domains of said at least two homologous distinct antigen components of said six or more homologous distinct antigen components share less than 80% sequence identity.
Embodiment 29. The vaccine composition of Embodiment 28, wherein said receptor binding domains of said at least two homologous distinct antigen components of said six or more homologous distinct antigen components share less than 75% sequence identity.
Embodiment 30. The vaccine composition of Embodiment 29, wherein said receptor binding domains of said at least two homologous distinct antigen components of said six or more homologous distinct antigen components share less than 70% sequence identity.
Embodiment 31. The vaccine composition of any one of Embodiment 1-Embodiment 30, wherein an administration of said vaccine composition reduces a dominant population of immunogenic single variant epitopes corresponding to said homologous distinct antigen components.
Embodiment 32. The vaccine composition of any one of Embodiment 1-Embodiment 31, wherein any 2 of said six or more homologous distinct antigen components share less than about 90% sequence identity.
Embodiment 33. The vaccine composition of Embodiment 31, wherein any 2 of said six or more homologous distinct antigen components share less than about 85% sequence identity.
Embodiment 34. The vaccine composition of Embodiment 33, wherein any 2 of said six or more homologous distinct antigen components share less than about 80% sequence identity.
Embodiment 35. The vaccine composition of Embodiment 34, wherein any 2 of said six or more homologous distinct antigen components share less than about 75% sequence identity.
Embodiment 36. The vaccine composition of Embodiment 35, wherein any 2 of said six or more homologous distinct antigen components share less than about 70% sequence identity.
Embodiment 37. The vaccine composition of any one of Embodiment 1-Embodiment 36, wherein said vaccine composition comprises 7 or more homologous distinct antigen components.
Embodiment 38. The vaccine composition of any one of Embodiment 1-Embodiment 37, wherein said vaccine composition comprises 10 or more homologous distinct antigen components.
Embodiment 39. The vaccine composition of any one of Embodiment 1-Embodiment 38, wherein said vaccine composition comprises 15 or more homologous distinct antigen components.
Embodiment 40. The vaccine composition of any one of Embodiment 1-Embodiment 39, wherein said vaccine composition comprises 20 or more homologous distinct antigen components.
Embodiment 41. The vaccine composition of any one of Embodiment 1-Embodiment 40, wherein said vaccine composition comprises 25 or more homologous distinct antigen components.
Embodiment 42. The vaccine composition of any one of Embodiment 1-Embodiment 41, wherein said vaccine composition comprises 30 or more homologous distinct antigen components.
Embodiment 43. The vaccine composition of any one of Embodiment 1-Embodiment 42, wherein any 3 of said six or more homologous distinct antigen components share less than 95% sequence identity.
Embodiment 44. The vaccine composition of any one of Embodiment 1-Embodiment 43, wherein any 4 of said six or more homologous distinct antigen components share less than 95% sequence identity.
Embodiment 45. The vaccine composition of any one of Embodiment 1-Embodiment 44, wherein any 5 of said six or more homologous distinct antigen components share less than 95% sequence identity.
Embodiment 46. The vaccine composition of any one of Embodiment 1-Embodiment 45, wherein any 6 of said six or more homologous distinct antigen components share less than 95% sequence identity.
Embodiment 47. The vaccine composition of any one of Embodiment 1-Embodiment 46, wherein said vaccine composition further comprises one or more non-homologous antigen components.
Embodiment 48. The vaccine composition of any one of Embodiment 1-Embodiment 47, wherein said vaccine composition further comprises an adjuvant.
Embodiment 49. The vaccine composition of any one of Embodiment 1-Embodiment 48, wherein said six or more homologous distinct antigen components are present in said vaccine composition at the same concentration/amount.
Embodiment 50. The vaccine composition of any one of Embodiment 1-Embodiment 49, wherein said six or more homologous distinct antigen components are present in said vaccine composition at distinct concentrations/amounts.
Embodiment 51. The vaccine composition of any one of Embodiment 1-Embodiment 50, wherein a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components.
Embodiment 52. The vaccine composition of any one of Embodiment 1-Embodiment 51, wherein a dose of a homologous distinct antigen components of said six or more homologous distinct antigen components is calculated to be proportional to its average phylogenetic distance from the other homologous distinct antigen components of said six or more homologous distinct antigen components.
Embodiment 53. The vaccine composition of any one of Embodiment 1-Embodiment 52, wherein said vaccine composition comprises a fragment of a SARS virus.
Embodiment 54. The vaccine composition of any one of Embodiment 1-Embodiment 53, wherein said vaccine composition comprises a fragment of an influenza virus.
Embodiment 55. The vaccine composition of any one of Embodiment 1-Embodiment 54, wherein said vaccine composition comprises a fragment of an HIV virus.
Embodiment 56. The vaccine composition of any one of Embodiment 1-Embodiment 55, wherein said vaccine composition comprises a fragment of a SARS1 virus.
Embodiment 57. A vaccine composition for human adults comprising six or more homologous distinct antigen components, wherein two of said six or more homologous distinct antigen components share less than 95% sequence identity, and
   a. wherein the homologous distinct antigen components comprise proteins, and wherein a concentration/amount of a protein in a dose of said human adult vaccine composition is about 1 nanogram (ng) to about 3 micrograms (μg);
   b. wherein the homologous distinct antigen components comprise a plurality of RNA, and wherein a concentration/amount of an RNA of said plurality of RNA in a dose of said human adult vaccine composition is about 1 ng to about 5 μg per dose; or
   c. wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said human adult vaccine composition is about 1 ng to about 5 μg per dose.
Embodiment 58. The vaccine composition of Embodiment 57, wherein said vaccine composition is for administration in an adult who is 25 years of age or older.
Embodiment 59. The vaccine composition of Embodiment 57, wherein said vaccine composition is for administration in an adult who is 50 years of age or older.
Embodiment 60. The vaccine composition of Embodiment 57, wherein said vaccine composition is for administration in an adult who is 75 years of age or older.
Embodiment 61. The vaccine composition of any one of Embodiment 57-Embodiment 60, wherein a concentration/amount of a homologous distinct antigen components component of said homologous distinct antigen components in a dose of said vaccine composition is about 1 nanogram (ng) to about 2 μg.
Embodiment 62. The vaccine composition of Embodiment 61, wherein a concentration/amount of a homologous distinct antigen components component of said homologous distinct antigen components in a dose of said vaccine composition is about 1 nanogram (ng) to about 1 microgram (μg).
Embodiment 63. The vaccine composition of Embodiment 62, wherein a concentration/amount of a homologous distinct antigen components component of said homologous distinct antigen components in a dose of said vaccine composition is about 1 nanogram (ng) to about 0.1 microgram (μg).
Embodiment 64. The vaccine composition of any one of Embodiment 57-Embodiment 63, wherein the homologous distinct antigen components comprise a component of a virus.
Embodiment 65. The vaccine composition of Embodiment 64, wherein the component of the virus is a receptor-binding domain.
Embodiment 66. The vaccine composition of Embodiment 64, wherein at least two homologous distinct antigen components of said six or more homologous distinct antigen components comprise receptor binding domains for a cell surface protein.
Embodiment 67. The vaccine composition of Embodiment 66, wherein said cell surface protein is mammalian.
Embodiment 68. The vaccine composition of Embodiment 66, wherein said receptor binding domains of said at least two homologous distinct antigen components of said six or more homologous distinct antigen components share less than 99% sequence identity.
Embodiment 69. The vaccine composition of Embodiment 68, wherein said receptor binding domains of said at least two homologous distinct antigen components of said six or more homologous distinct antigen components share less than 98% sequence identity.
Embodiment 70. The vaccine composition of Embodiment 69, wherein said receptor binding domains of said at least two homologous distinct antigen components of said six or more homologous distinct antigen components share less than 95% sequence identity.
Embodiment 71. The vaccine composition of Embodiment 70, wherein said receptor binding domains of said at least two homologous distinct antigen components of said six or more homologous distinct antigen components share less than 95% sequence identity.
Embodiment 72. The vaccine composition of Embodiment 71, wherein said receptor binding domains of said at least two homologous distinct antigen components of said six or more homologous distinct antigen components share less than 90% sequence identity.
Embodiment 73. The vaccine composition of Embodiment 72, wherein said receptor binding domains of said at least two homologous distinct antigen components of said six or more homologous distinct antigen components share less than 85% sequence identity.
Embodiment 74. The vaccine composition of Embodiment 73, wherein said receptor binding domains of said at least two homologous distinct antigen components of said six or more homologous distinct antigen components share less than 80% sequence identity.
Embodiment 75. The vaccine composition of Embodiment 74, wherein said receptor binding domains of said at least two homologous distinct antigen components of said six or more homologous distinct antigen components share less than 75% sequence identity.
Embodiment 76. The vaccine composition of Embodiment 75, wherein said receptor binding domains of said at least two homologous distinct antigen components of said six or more homologous distinct antigen components share less than 70% sequence identity.
Embodiment 77. The vaccine composition of any one of Embodiment 57-Embodiment 76, wherein an administration of said vaccine composition reduces a dominant population of immunogenic single variant epitopes corresponding to said homologous distinct antigen components.
Embodiment 78. The vaccine composition of any one of Embodiment 57-Embodiment 77, wherein any 2 of said six or more homologous distinct antigen components share less than about 90% sequence identity.
Embodiment 79. The vaccine composition of Embodiment 78, wherein any 2 of said six or more homologous distinct antigen components share less than about 85% sequence identity.
Embodiment 80. The vaccine composition of Embodiment 79, wherein any 2 of said six or more homologous distinct antigen components share less than about 80% sequence identity.
Embodiment 81. The vaccine composition of Embodiment 80, wherein any 2 of said six or more homologous distinct antigen components share less than about 75% sequence identity.
Embodiment 82. The vaccine composition of Embodiment 81, wherein any 2 of said six or more homologous distinct antigen components share less than about 70% sequence identity.
Embodiment 83. The vaccine composition of any one of Embodiment 57-Embodiment 82, wherein said vaccine composition comprises 7 or more homologous distinct antigen components.
Embodiment 84. The vaccine composition of any one of Embodiment 57-Embodiment 83, wherein said vaccine composition comprises 10 or more homologous distinct antigen components.
Embodiment 85. The vaccine composition of any one of Embodiment 57-Embodiment 84, wherein said vaccine composition comprises 15 or more homologous distinct antigen components.
Embodiment 86. The vaccine composition of any one of Embodiment 57-Embodiment 85, wherein said vaccine composition comprises 20 or more homologous distinct antigen components.
Embodiment 87. The vaccine composition of any one of Embodiment 57-Embodiment 86, wherein said vaccine composition comprises 25 or more homologous distinct antigen components.
Embodiment 88. The vaccine composition of any one of Embodiment 57-Embodiment 87, wherein said vaccine composition comprises 30 or more homologous distinct antigen components.
Embodiment 89. The vaccine composition of any one of Embodiment 57-Embodiment 87, wherein any 3 of said six or more homologous distinct antigen components share less than 95% sequence identity.
Embodiment 90. The vaccine composition of any one of Embodiment 57-Embodiment 87, wherein any 4 of said six or more homologous distinct antigen components share less than 95% sequence identity.
Embodiment 91. The vaccine composition of any one of Embodiment 57-Embodiment 87, wherein any 5 of said six or more homologous distinct antigen components share less than 95% sequence identity.
Embodiment 92. The vaccine composition of any one of Embodiment 57-Embodiment 87, wherein any 6 of said six or more homologous distinct antigen components share less than 95% sequence identity.
Embodiment 93. The vaccine composition of any one of Embodiment 57-Embodiment 87, wherein any 2 of said six or more homologous distinct antigen components share at least about 95% sequence identity.
Embodiment 94. The vaccine composition of any one of Embodiment 57-Embodiment 87, wherein any 3 of said six or more homologous distinct antigen components share at least about 95% sequence identity.
Embodiment 95. The vaccine composition of any one of Embodiment 57-Embodiment 94, wherein any 4 of said six or more homologous distinct antigen components share at least about 95% sequence identity.
Embodiment 96. The vaccine composition of any one of Embodiment 57-Embodiment 95, wherein said vaccine composition further comprises one or more non-homologous antigen components.
Embodiment 97. The vaccine composition of any one of Embodiment 57-Embodiment 96, wherein said vaccine composition further comprises an adjuvant.
Embodiment 98. The vaccine composition of any one of Embodiment 57-Embodiment 97, wherein said six or more homologous distinct antigen components are present in said vaccine composition at the same concentration/amount.
Embodiment 99. The vaccine composition of any one of Embodiment 57-Embodiment 98, wherein said six or more homologous distinct antigen components are present in said vaccine composition at distinct concentrations/amounts.
Embodiment 100. The vaccine composition of any one of Embodiment 57-Embodiment 99, wherein a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components.
Embodiment 101. The vaccine composition of any one of Embodiment 57-Embodiment 100, wherein a dose of a homologous distinct antigen components of said six or more homologous distinct antigen components is calculated to be proportional to its average phylogenetic distance from the other homologous distinct antigen components of said six or more homologous distinct antigen components.
Embodiment 102. The vaccine composition of any one of Embodiment 57-Embodiment 101, wherein said vaccine composition comprises a fragment of a SARS virus.
Embodiment 103. The vaccine composition of any one of Embodiment 57-Embodiment 102, wherein said vaccine composition comprises a fragment of an influenza virus.
Embodiment 104. The vaccine composition of any one of Embodiment 57-Embodiment 103, wherein said vaccine composition comprises a fragment of an HIV virus.
Embodiment 105. The vaccine composition of any one of Embodiment 57-Embodiment 104, wherein said vaccine composition comprises a fragment of a SARS1 virus.
Embodiment 106. A vaccine composition comprising six or more homologous distinct antigen components, wherein two of said six or more homologous distinct antigen components share less than 95% sequence identity, and
   a. wherein the homologous distinct antigen components comprise proteins, and wherein a concentration/amount of a protein in a dose of said vaccine composition is about 1 nanogram (ng) to about 1 microgram (μg);
   b. wherein the homologous distinct antigen components comprise a plurality of RNA, and wherein a concentration/amount of an RNA of said plurality of RNA in a dose of said vaccine composition is about 1 ng to about 2.5 μg per dose; or
   c. wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said vaccine composition is about 1 ng to about 2.5 μg per dose,
wherein the vaccine composition is a human pediatric vaccine composition.
Embodiment 107. The vaccine composition of Embodiment 106, wherein said vaccine composition is for administration in a child between the ages of about 1 day old and about 18 years old.
Embodiment 108. The vaccine composition of Embodiment 106, wherein said vaccine composition is for administration in a child between the ages of about 1 day old and about 5 years old.
Embodiment 109. The vaccine composition of Embodiment 106, wherein said vaccine composition is for administration in a child between the ages of about 5 years old and about 18 years old.
Embodiment 110. The vaccine composition of any one of Embodiment 106-Embodiment 109, wherein a concentration/amount of a homologous distinct antigen components component of said homologous distinct antigen components in a dose of said vaccine composition is about 1 nanogram (ng) to about 0.5 micrograms (μg).
Embodiment 111. The vaccine composition of Embodiment 106 wherein a concentration/amount of a homologous distinct antigen components component of said homologous distinct antigen components in a dose of said vaccine composition is about 1 nanogram (ng) to about 0.1 micrograms (μg).
Embodiment 112. The vaccine composition of any one of Embodiment 106-Embodiment 111, wherein the homologous distinct antigen components comprise a component of a virus.
Embodiment 113. The vaccine composition of Embodiment 112, wherein the component of the virus is a receptor-binding domain.
Embodiment 114. The vaccine composition of Embodiment 113, wherein at least two homologous distinct antigen components of said six or more homologous distinct antigen components comprise receptor binding domains for a cell surface protein.
Embodiment 115. The vaccine composition of Embodiment 114, wherein said cell surface protein is mammalian.
Embodiment 116. The vaccine composition of Embodiment 113, wherein said receptor binding domains of said at least two homologous distinct antigen components of said six or more homologous distinct antigen components share less than 99% sequence identity.
Embodiment 117. The vaccine composition of Embodiment 113, wherein said receptor binding domains of said at least two homologous distinct antigen components of said six or more homologous distinct antigen components share less than 98% sequence identity.
Embodiment 118. The vaccine composition of Embodiment 113, wherein said receptor binding domains of said at least two homologous distinct antigen components of said six or more homologous distinct antigen components share less than 95% sequence identity.
Embodiment 119. The vaccine composition of Embodiment 113, wherein said receptor binding domains of said at least two homologous distinct antigen components of said six or more homologous distinct antigen components share less than 95% sequence identity.
Embodiment 120. The vaccine composition of Embodiment 113, wherein said receptor binding domains of said at least two homologous distinct antigen components of said six or more homologous distinct antigen components share less than 90% sequence identity.
Embodiment 121. The vaccine composition of Embodiment 113, wherein said receptor binding domains of said at least two homologous distinct antigen components of said six or more homologous distinct antigen components share less than 85% sequence identity.
Embodiment 122. The vaccine composition of Embodiment 113, wherein said receptor binding domains of said at least two homologous distinct antigen components of said six or more homologous distinct antigen components share less than 80% sequence identity.
Embodiment 123. The vaccine composition of Embodiment 113, wherein said receptor binding domains of said at least two homologous distinct antigen components of said six or more homologous distinct antigen components share less than 75% sequence identity.
Embodiment 124. The vaccine composition of Embodiment 113, wherein said receptor binding domains of said at least two homologous distinct antigen components of said six or more homologous distinct antigen components share less than 70% sequence identity.
Embodiment 125. The vaccine composition of any one of Embodiment 106-Embodiment 124, wherein an administration of said vaccine composition reduces a dominant population of immunogenic single variant epitopes corresponding to said homologous distinct antigen components.
Embodiment 126. The vaccine composition of any one of Embodiment 106-Embodiment 125, wherein any 2 of said six or more homologous distinct antigen components share less than about 90% sequence identity.
Embodiment 127. The vaccine composition of Embodiment 126, wherein any 2 of said six or more homologous distinct antigen components share less than about 85% sequence identity.
Embodiment 128. The vaccine composition of Embodiment 127, wherein any 2 of said six or more homologous distinct antigen components share less than about 80% sequence identity.
Embodiment 129. The vaccine composition of Embodiment 128, wherein any 2 of said six or more homologous distinct antigen components share less than about 75% sequence identity.
Embodiment 130. The vaccine composition of Embodiment 129, wherein any 2 of said six or more homologous distinct antigen components share less than about 70% sequence identity.
Embodiment 131. The vaccine composition of any one of Embodiment 106-Embodiment 130, wherein said vaccine composition comprises 7 or more homologous distinct antigen components.
Embodiment 132. The vaccine composition of any one of Embodiment 106-Embodiment 131, wherein said vaccine composition comprises 10 or more homologous distinct antigen components.
Embodiment 133. The vaccine composition of any one of Embodiment 106-Embodiment 132, wherein said vaccine composition comprises 15 or more homologous distinct antigen components.
Embodiment 134. The vaccine composition of any one of Embodiment 106-Embodiment 133, wherein said vaccine composition comprises 20 or more homologous distinct antigen components.
Embodiment 135. The vaccine composition of any one of Embodiment 106-Embodiment 134, wherein said vaccine composition comprises 25 or more homologous distinct antigen components.
Embodiment 136. The vaccine composition of any one of Embodiment 106-Embodiment 135, wherein said vaccine composition comprises 30 or more homologous distinct antigen components.
Embodiment 137. The vaccine composition of any one of Embodiment 106-Embodiment 136, wherein any 3 of said six or more homologous distinct antigen components share less than 95% sequence identity.
Embodiment 138. The vaccine composition of any one of Embodiment 106-Embodiment 137, wherein any 4 of said six or more homologous distinct antigen components share less than 95% sequence identity.
Embodiment 139. The vaccine composition of any one of Embodiment 106-Embodiment 138, wherein any 5 of said six or more homologous distinct antigen components share less than 95% sequence identity.
Embodiment 140. The vaccine composition of any one of Embodiment 106-Embodiment 139, wherein any 6 of said six or more homologous distinct antigen components share less than 95% sequence identity.
Embodiment 141. The vaccine composition of any one of Embodiment 106-Embodiment 140, wherein any 2 of said six or more homologous distinct antigen components share at least about 95% sequence identity.
Embodiment 142. The vaccine composition of any one of Embodiment 106-Embodiment 141, wherein any 3 of said six or more homologous distinct antigen components share at least about 95% sequence identity.
Embodiment 143. The vaccine composition of any one of Embodiment 106-Embodiment 142, wherein any 4 of said six or more homologous distinct antigen components share at least about 95% sequence identity.
Embodiment 144. The vaccine composition of any one of Embodiment 106-Embodiment 143, wherein said vaccine composition further comprises one or more non-homologous antigen components.
Embodiment 145. The vaccine composition of any one of Embodiment 106-Embodiment 144, wherein said vaccine composition further comprises an adjuvant.
Embodiment 146. The vaccine composition of any one of Embodiment 106-Embodiment 145, wherein said six or more homologous distinct antigen components are present in said vaccine composition at the same concentration/amount.
Embodiment 147. The vaccine composition of any one of Embodiment 106-Embodiment 146, wherein said six or more homologous distinct antigen components are present in said vaccine composition at distinct concentrations/amounts.
Embodiment 148. The vaccine composition of any one of Embodiment 106-Embodiment 147, wherein a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components.
Embodiment 149. The vaccine composition of any one of Embodiment 106-Embodiment 148, wherein a dose of a homologous distinct antigen components of said six or more homologous distinct antigen components is calculated to be proportional to its average phylogenetic distance from the other homologous distinct antigen components of said six or more homologous distinct antigen components.
Embodiment 150. The vaccine composition of any one of Embodiment 106-Embodiment 149, wherein said vaccine composition comprises a fragment of a SARS virus.
Embodiment 151. The vaccine composition of any one of Embodiment 106-Embodiment 150, wherein said vaccine composition comprises a fragment of an influenza virus.
Embodiment 152. The vaccine composition of any one of Embodiment 106-Embodiment 151, wherein said vaccine composition comprises a fragment of an HIV virus.
Embodiment 153. The vaccine composition of any one of Embodiment 106-Embodiment 152, wherein said vaccine composition comprises a fragment of a SARS1 virus.
Embodiment 154. A vaccine composition comprising six or more homologous distinct antigen components, wherein two of said six or more homologous distinct antigen components share less than 95% sequence identity, and
   a. wherein the homologous distinct antigen components comprise proteins, and wherein a concentration/amount of a protein in a dose of said vaccine composition is about 1 nanogram (ng) to about 3 microgram (μg);
   b. wherein the homologous distinct antigen components comprise a plurality of RNA, and wherein a concentration/amount of an RNA of said plurality of RNA in a dose of said vaccine composition is about 1 ng to about 5 μg per dose; or
   c. wherein the homologous distinct antigen components comprise a plurality of proteins displayed on heterologous viral-like particles (VLPs), and wherein a concentration/amount of a protein displayed on a heterologous VLP of said plurality of proteins displayed on heterologous VLPs in a dose of said vaccine composition is about 1 ng to about 5 μg per dose.
wherein the vaccine composition is a veterinary vaccine composition.
Embodiment 155. The vaccine composition of Embodiment 154, wherein said vaccine composition is for administration in a livestock animal.
Embodiment 156. The vaccine composition of Embodiment 155, wherein said vaccine composition is for administration in a cow.
Embodiment 157. The vaccine composition of Embodiment 155, wherein said vaccine composition is for administration in a sheep.
Embodiment 158. The vaccine composition of Embodiment 155, wherein said vaccine composition is for administration in a pig.
Embodiment 159. The vaccine composition of Embodiment 154, wherein said vaccine composition is for administration in a bird.
Embodiment 160. The vaccine composition of Embodiment 154, wherein said vaccine composition is for administration in a canine animal.
Embodiment 161. The vaccine composition of Embodiment 154, wherein said vaccine composition is for administration in a feline animal.
Embodiment 162. The vaccine composition of any one of Embodiment 154-Embodiment 161, wherein a concentration/amount of a homologous distinct antigen components component of said homologous distinct antigen components in a dose of said vaccine composition is about 1 nanogram (ng) to about 3 micrograms (μg).
Embodiment 163. The vaccine composition of Embodiment 162, wherein a concentration/amount of a homologous distinct antigen components component of said homologous distinct antigen components in a dose of said vaccine composition is about 1 nanogram (ng) to about 1 microgram (μg).
Embodiment 164. The vaccine composition of Embodiment 163, wherein a concentration/amount of a homologous distinct antigen components component of said homologous distinct antigen components in a dose of said vaccine composition is about 1 nanogram (ng) to about 0.1 microgram (μg).
Embodiment 165. The vaccine composition of any one of Embodiment 154-Embodiment 164, wherein the homologous distinct antigen components comprise a component of a virus.
Embodiment 166. The vaccine composition of Embodiment 165, wherein the component of the virus is a receptor-binding domain.
Embodiment 167. The vaccine composition of Embodiment 166, wherein at least two homologous distinct antigen components of said six or more homologous distinct antigen components comprise receptor binding domains for a cell surface protein.
Embodiment 168. The vaccine composition of Embodiment 167, wherein said cell surface protein is mammalian.
Embodiment 169. The vaccine composition of Embodiment 167, wherein said receptor binding domains of said at least two homologous distinct antigen components of said six or more homologous distinct antigen components share less than 99% sequence identity.
Embodiment 170. The vaccine composition of Embodiment 169, wherein said receptor binding domains of said at least two homologous distinct antigen components of said six or more homologous distinct antigen components share less than 98% sequence identity.
Embodiment 171. The vaccine composition of Embodiment 169, wherein said receptor binding domains of said at least two homologous distinct antigen components of said six or more homologous distinct antigen components share less than 95% sequence identity.
Embodiment 172. The vaccine composition of Embodiment 169, wherein said receptor binding domains of said at least two homologous distinct antigen components of said six or more homologous distinct antigen components share less than 95% sequence identity.
Embodiment 173. The vaccine composition of Embodiment 169, wherein said receptor binding domains of said at least two homologous distinct antigen components of said six or more homologous distinct antigen components share less than 90% sequence identity.
Embodiment 174. The vaccine composition of Embodiment 169, wherein said receptor binding domains of said at least two homologous distinct antigen components of said six or more homologous distinct antigen components share less than 85% sequence identity.
Embodiment 175. The vaccine composition of Embodiment 169, wherein said receptor binding domains of said at least two homologous distinct antigen components of said six or more homologous distinct antigen components share less than 80% sequence identity.
Embodiment 176. The vaccine composition of Embodiment 169, wherein said receptor binding domains of said at least two homologous distinct antigen components of said six or more homologous distinct antigen components share less than 75% sequence identity.
Embodiment 177. The vaccine composition of Embodiment 169, wherein said receptor binding domains of said at least two homologous distinct antigen components of said six or more homologous distinct antigen components share less than 70% sequence identity.
Embodiment 178. The vaccine composition of any one of Embodiment 154-Embodiment 177, wherein an administration of said vaccine composition reduces a dominant population of immunogenic single variant epitopes corresponding to said homologous distinct antigen components.
Embodiment 179. The vaccine composition of any one of Embodiment 154-Embodiment 178, wherein any 2 of said six or more homologous distinct antigen components share less than about 90% sequence identity.
Embodiment 180. The vaccine composition of Embodiment 179, wherein any 2 of said six or more homologous distinct antigen components share less than about 85% sequence identity.
Embodiment 181. The vaccine composition of Embodiment 180, wherein any 2 of said six or more homologous distinct antigen components share less than about 80% sequence identity.
Embodiment 182. The vaccine composition of Embodiment 181, wherein any 2 of said six or more homologous distinct antigen components share less than about 75% sequence identity.
Embodiment 183. The vaccine composition of Embodiment 182, wherein any 2 of said six or more homologous distinct antigen components share less than about 70% sequence identity.
Embodiment 184. The vaccine composition of any one of Embodiment 154-Embodiment 183, wherein said vaccine composition comprises 7 or more homologous distinct antigen components.
Embodiment 185. The vaccine composition of any one of Embodiment 154-Embodiment 184, wherein said vaccine composition comprises 10 or more homologous distinct antigen components.
Embodiment 186. The vaccine composition of any one of Embodiment 154-Embodiment 185, wherein said vaccine composition comprises 15 or more homologous distinct antigen components.
Embodiment 187. The vaccine composition of any one of Embodiment 154-Embodiment 186, wherein said vaccine composition comprises 20 or more homologous distinct antigen components.
Embodiment 188. The vaccine composition of any one of Embodiment 154-Embodiment 187, wherein said vaccine composition comprises 25 or more homologous distinct antigen components.
Embodiment 189. The vaccine composition of any one of Embodiment 154-Embodiment 188, wherein said vaccine composition comprises 30 or more homologous distinct antigen components.
Embodiment 190. The vaccine composition of any one of Embodiment 154-Embodiment 189, wherein any 3 of said six or more homologous distinct antigen components share less than 95% sequence identity.
Embodiment 191. The vaccine composition of any one of Embodiment 154-Embodiment 190, wherein any 4 of said six or more homologous distinct antigen components share less than 95% sequence identity.
Embodiment 192. The vaccine composition of any one of Embodiment 154-Embodiment 191, wherein any 5 of said six or more homologous distinct antigen components share less than 95% sequence identity.
Embodiment 193. The vaccine composition of any one of Embodiment 154-Embodiment 192, wherein any 6 of said six or more homologous distinct antigen components share less than 95% sequence identity.
Embodiment 194. The vaccine composition of any one of Embodiment 154-Embodiment 193, wherein any 2 of said six or more homologous distinct antigen components share at least about 95% sequence identity.
Embodiment 195. The vaccine composition of any one of Embodiment 154-Embodiment 194, wherein any 3 of said six or more homologous distinct antigen components share at least about 95% sequence identity.
Embodiment 196. The vaccine composition of any one of Embodiment 154-Embodiment 195, wherein any 4 of said six or more homologous distinct antigen components share at least about 95% sequence identity.
Embodiment 197. The vaccine composition of any one of Embodiment 154-Embodiment 196, wherein said vaccine composition further comprises one or more non-homologous antigen components.
Embodiment 198. The vaccine composition of any one of Embodiment 154-Embodiment 197, wherein said vaccine composition further comprises an adjuvant.
Embodiment 199. The vaccine composition of any one of Embodiment 154-Embodiment 198, wherein said six or more homologous distinct antigen components are present in said vaccine composition at the same concentration/amount.
Embodiment 200. The vaccine composition of any one of Embodiment 154-Embodiment 199, wherein said six or more homologous distinct antigen components are present in said vaccine composition at distinct concentrations/amounts.
Embodiment 201. The vaccine composition of any one of Embodiment 154-Embodiment 200, wherein a dose of a homologous distinct antigen components of said six or more homologous distinct antigen components is calculated to be proportional to the phylogenetic distance of the homologous distinct antigen components to another homologous distinct antigen components, wherein said another homologous distinct antigen components has the least distance to said homologous distinct antigen components out of said six or more homologous distinct antigen components.
Embodiment 202. The vaccine composition of any one of Embodiment 154-Embodiment 201, wherein a dose of a homologous distinct antigen components of said six or more homologous distinct antigen components is calculated to be proportional to its average phylogenetic distance from the other homologous distinct antigen components of said six or more homologous distinct antigen components.
Embodiment 203. The vaccine composition of any one of Embodiment 154-Embodiment 202, wherein said vaccine composition comprises a fragment of a SARS virus.
Embodiment 204. The vaccine composition of any one of Embodiment 154-Embodiment 203, wherein said vaccine composition comprises a fragment of an influenza virus.
Embodiment 205. The vaccine composition of any one of Embodiment 154-Embodiment 204, wherein said vaccine composition comprises a fragment of an HIV virus.
Embodiment 206. The vaccine composition of any one of Embodiment 154-Embodiment 205, wherein said vaccine composition comprises a fragment of a SARS1 virus.
Embodiment 207. A method of inducing an immune response against a pathogen in a subject comprising administering the vaccine composition of any one of Embodiment 1-Embodiment 206.
Embodiment 208. The method of Embodiment 207, where the method is for prophylaxis against disease caused by the pathogen.

### EXAMPLES

### Example 1: Serum Activity to mRNA Antigens in Mice

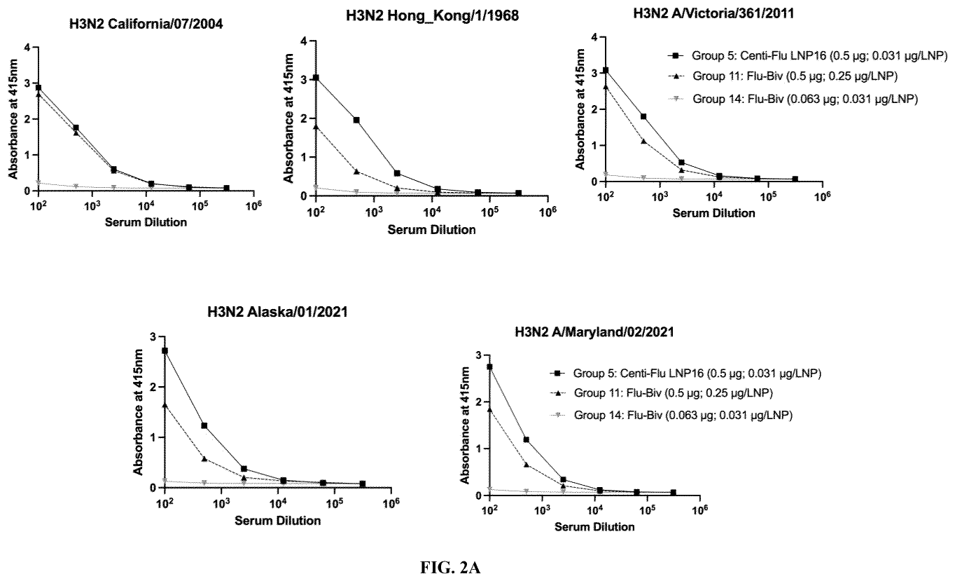

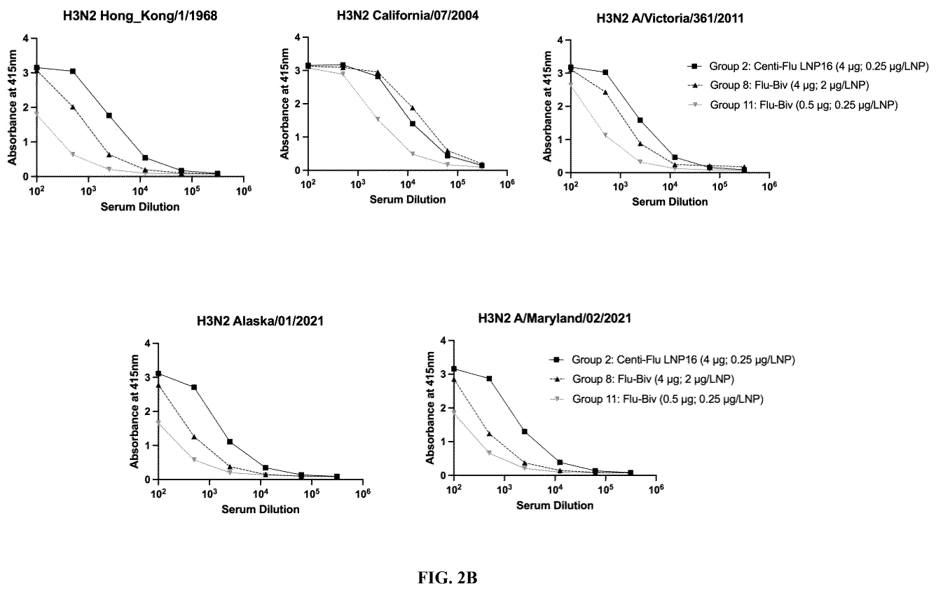

Mice were injected with either Centi-Flu (mRNA encoding a mixture of 16 hemagglutinin antigens, including eight H1, corresponding to SEQ ID NOs. 4, 6, 8, 10, 12, 14, 16, 18; and eight H3, corresponding to SEQ ID NOs. 20, 22, 24, 26, 28, 30, 32, 34) or Flu-Biv (mRNA encoding a mixture of 2 hemagglutinin antigens, including one H1 (SEQ ID NO. 10) and one H3 (SEQ ID NO. 22)). All mRNA vaccine compositions were complexed in lipid nanoparticles (LNPs). Specifically, the same LNP composition as BNT162b2 was used. The serum was collected 16 days after immunization and measured by ELISA for antibodies that bind to recombinant hemagglutinin proteins of H3N2 Hong Kong/i/1968 (present in Centi-Flu), H3N2 California/07/2004 (present in Centi-Flu and Flu-Biv), H3N2 Alaska/01/2021 (present in Centi-Flu), H3N2 A/Victoria/361/2011 (heterologous to both Centi-Flu and Flu-Biv), or H3N2 A/Maryland/02/2021 (heterologous to both Centi-Flu and Flu-Biv). FIG. 2A suggests that at a lower matched per-antigen dose, Flu-Biv induced no response while Centi-Flu induced a robust response. For example, when the mice were injected with Centi-Flu LNP at 0.031 μg per antigen (=0.5 ug total mRNA) or Flu-Biv at 0.031 μg per antigen (=0.063 ug total mRNA), Flu-Biv induced no response, while Centi-Flu induced a robust response. Furthermore, this difference cannot be explained by the total antigen dose, as Flu-Biv at 0.25 ug per antigen (=0.5 ug total mRNA) still exhibits a weaker response in all unmatched antigens than Centi-Flu at 0.031 μg/antigen (=0.5 ug total mRNA). FIG. 2B suggests that at a higher per-antigen dose, Flu-Biv induced a weak response, while Centi-Flu induced a more potent response. For example, when the mice were injected with Centi-Flu LNP at 0.25 μg per antigen (=4 ug total mRNA) or Flu-Biv at 0.25 μg per antigen (=0.5 ug total mRNA), Flu-Biv induced a weak response, while Centi-Flu induced a robust response. Furthermore, this difference cannot be explained by the total antigen dose, as Flu-Biv at 2 μg per antigen (=4 ug total mRNA) still exhibits a weaker response in all unmatched antigens than Centi-Flu at 0.25 ug/antigen (=4 ug total mRNA). Therefore it is the unique combination of (1) using six or more homologous distinct antigen components and (2) mRNA encoding each component at a low dose, that achieves the desired serological effect.

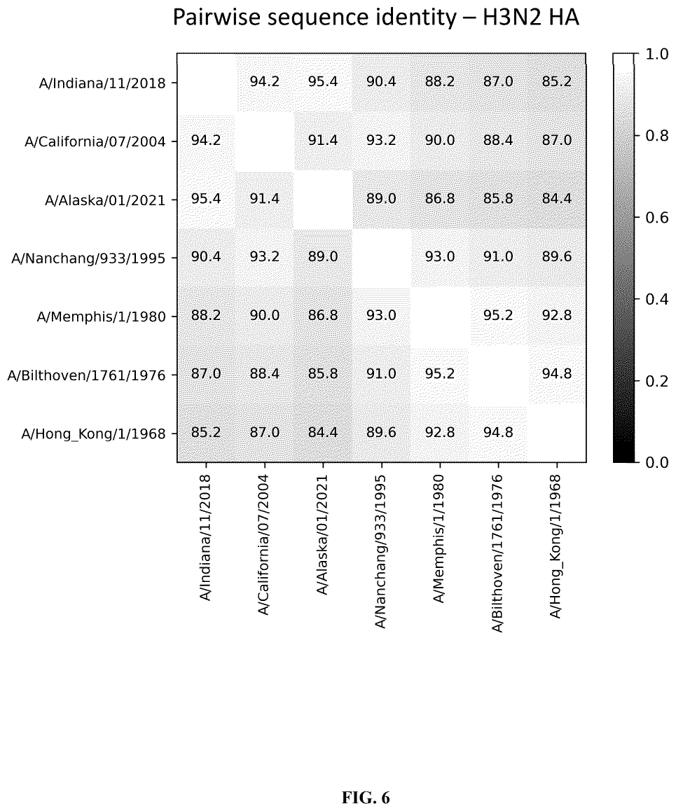

One exemplary set of eight homologous distinct antigen components encoded by mRNA in the Centi-Flu vaccine composition are A/Hong Kong/1/1968 (SEQ ID NO. 33), A/Nanchang/933/1995 (SEQ ID NO. 27), A/California/07/2004 (SEQ ID NO. 21), A/Memphis/1/1980 (SEQ ID NO. 29), A/Alaska/01/2021 (SEQ ID NO. 23), A/Indiana/11/2018 (SEQ ID NO. 19), A/Cambodia/e0826360/2020 (SEQ ID NO. 25), and A/Bilthoven/1971/1976 (SEQ ID NO. 31). FIG. 6 shows that any two of these eight H3N2 homologous distinct antigen components have pairwise sequence identity less than 96%, and greater than 80%. Any subset of six of these eight constitute sets of six homologous distinct antigen components with pairwise sequence identity less than 96% and greater than 80%.

mRNA generation from DNA plasmids and subsequent encapsulation in lipid nanoparticles (LNPs) was performed using methods well-known in the art, summarized here. DNA plasmids were constructed that comprise, under the SP6 promoter, the same 5′ UTR as used in the clinically approved BNT162b2 vaccine (SEQ ID NO: 1), DNA encoding an antigen component, the same 3′ UTR as used in BNT162b2 (SEQ ID NO: 2), and a poly-adenosine(120) tail. The plasmids were linearized using a NotI restriction site and utilized as templates for in vitro transcription (IVT) using SP6 RNA polymerase and a mix of nucleoside triphosphates. N1-methyl-pseudouridine-5′-triphosphate was used instead of uridine-5′-triphosphate for IVT. The Vaccinia enzymatic capping system (New England BioLabs) was used to generate capped mRNA. Purified mRNA was further encapsulated in the same LNP formulation as BNT162b2 (46.3% ionizable lipid ALC-0315, 9.4% phospholipid DSPC, 1.6% ALC-0159 PEG-lipid, and 42.7% cholesterol) to obtain the LNP-encapsulated RNA.

Serum ELISA assays in all Examples were performed as follows: The indicated proteins (5 ug/mL) were added to microtiter plates (CoStar), in coating buffer (0.1 M sodium bicarbonate, pH 8.6). After incubation at 4° C. overnight and blocking with 3% bovine serum albumin (BSA) in PBS, for 1 hour at 37° C., serially diluted serum/plasma in blocking buffer was added to individual wells and incubated for 1 hour at 37° C. Then, plates were washed three times with 0.05% PBST. Horseradish peroxidase (HRP)-conjugated anti-IgG secondary antibody was added to wells and incubated for 1 hour at 37° C. After washing three times with PBST, 2,2′-azino-bis-3-ethylbenzothiazoline-6-sulfonic (Thermo Fisher Scientific Inc. Waltham, MA, USA) or 3,3′,5,5′-tetramethylbenzidineliquid substrate system (Thermo Fisher Scientific Inc.) was added to the wells. Absorbance was measured at 405 or 650 nm, respectively, using a microplate spectrophotometer (Multiskan GO, Thermo Fisher Scientific Inc.).

### Example 2: Serum Activity to mRNA Antigens in Ferret

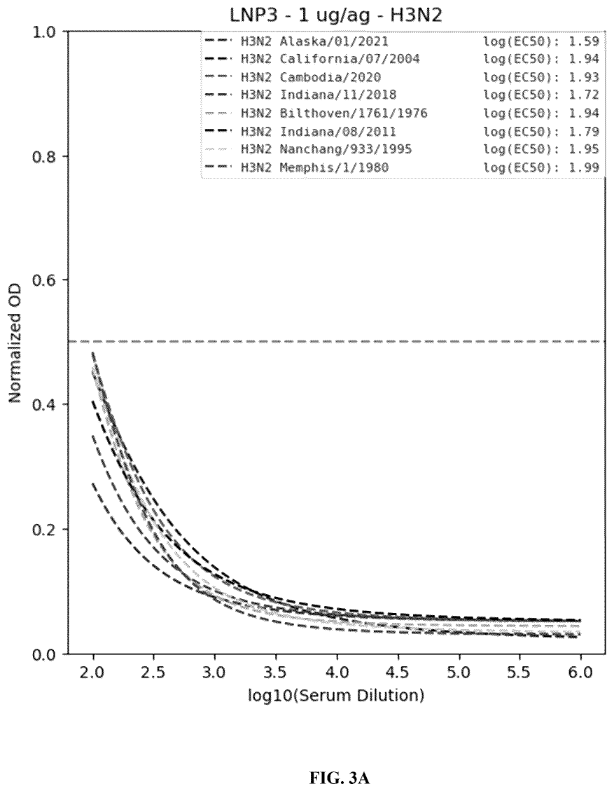

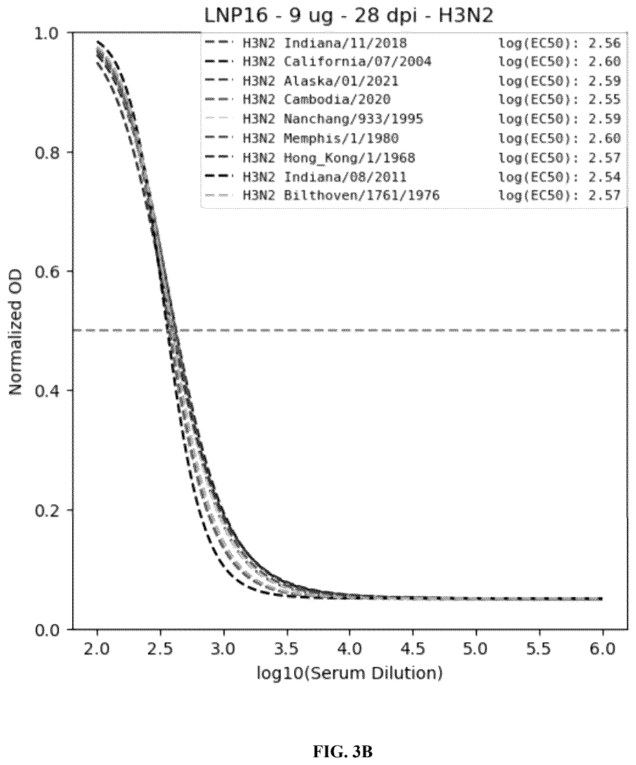

Ferrets were immunized once with mRNA encoding any one of eight distinct antigens complexed with a lipid nanoparticle (LNP). Specifically, ferrets were immunized with 1 μg of LNP-encapsulated mRNA encoding the hemagglutinin corresponding to H3N2 Alaska/01/2021 (SEQ ID NO: 24), H3N2 California/07/2004 (SEQ ID NO: 22), H3N2 Cambodia/2020 (SEQ ID NO: 26), H3N2 Indiana/11/2018 (SEQ ID NO: 20), H3N2 Bilthoven/1761/1976 (SEQ ID NO: 32), H3N2 Nanchang/933/1995 (SEQ ID NO: 28), or H3N2 Memphis/i/1980 (SEQ ID NO: 30). Serum was obtained 28 days after immunization. Serum reactivity to the recombinant protein of each antigen with which the ferrets were immunized was measured, as shown in FIG. 3A. The antigen-matched serum reactivity (i.e., serum from mice immunized with a given antigen tested against that same antigen) was very weak in all cases (EC50 not attained in any group even at the highest tested serum dilution of 1:100).

Ferrets were immunized with LNP-encapsulated mRNAs encoding the hemagglutinin corresponding to 8 H3N2 antigens (SEQ ID NOs: 24, 32, 22, 26, 20, 34, 28, 30), each at about 0.5 μg per antigen (total of 4 μg of H3N2 antigens). Serum reactivity to the recombinant protein of each antigen with which the ferrets were immunized was measured, as shown in FIG. 3B. The Centi-Flu immunized ferrets exhibited potent responses against all immunized antigens, as shown in FIG. 3B. It suggests that the single antigen at a low per-antigen dose (1 ug per antigen) is insufficient, but Centi-Flu at an even lower per-antigen dose (0.5 ug per antigen) induces potent and broad reactivity. Therefore, it is the unique combination of (1) using six or more homologous distinct antigen components and (2) mRNA encoding each component at a low dose, that achieves the desired serological effect.

All LNP-encapsulated RNA constructs were generated as described in Example 1. Serum ELISA was performed as described in Example 1.

One exemplary set of eight homologous distinct antigen components encoded by mRNA in the Centi-Flu vaccine composition are A/Hong Kong/1/1968 (SEQ ID NO. 33), A/Nanchang/933/1995 (SEQ ID NO. 27), A/California/07/2004 (SEQ ID NO. 21), A/Memphis/1/1980 (SEQ ID NO. 29), A/Alaska/01/2021 (SEQ ID NO. 23), A/Indiana/11/2018 (SEQ ID NO. 19), A/Cambodia/e0826360/2020 (SEQ ID NO. 25), and A/Bilthoven/1971/1976 (SEQ ID NO. 31). FIG. 6 shows that any two of these eight H3N2 homologous distinct antigen components have pairwise sequence identity less than 96%, and greater than 80%. Any subset of six of these eight constitute sets of six homologous distinct antigen components with pairwise sequence identity less than 96% and greater than 80%.

### Example 3: Serum Activity to HIV Protein Antigens in Mice

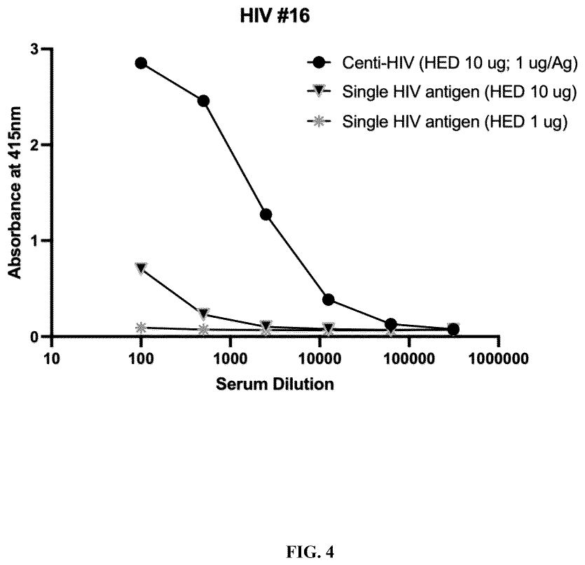

Centi-HIV is a mixture of gp120/gp41 recombinant fusion proteins (joined by a flexible glycine-serine linker) from 10 distinct HIV antigens. Mice were immunized twice (day 0, day 14) with either Centi-HIV, or a single gp120/gp41 recombinant fusion protein (“Single HIV antigen”). Serum was obtained at day 28. Serum reactivity against the same fusion protein used in “Single HIV antigen” is shown in FIG. 4. FIG. 4 demonstrates that at a per-antigen human-equivalent (i.e., allometrically scaled) dose of 1 ug (=total human-equivalent dose (“HED”) of 10 ug), Centi-HIV results in a potent response while Single HIV antigen at 1 ug exhibits no reactivity even against the same antigen used for immunization. This cannot be explained by total antigen dose, as Single HIV antigen at 10 ug still mounts a deficient response compared to Centi-HIV. Therefore, it is the unique combination of (1) using six or more homologous distinct antigen components and (2) each component being administered a low dose, that achieves the desired serological effect.

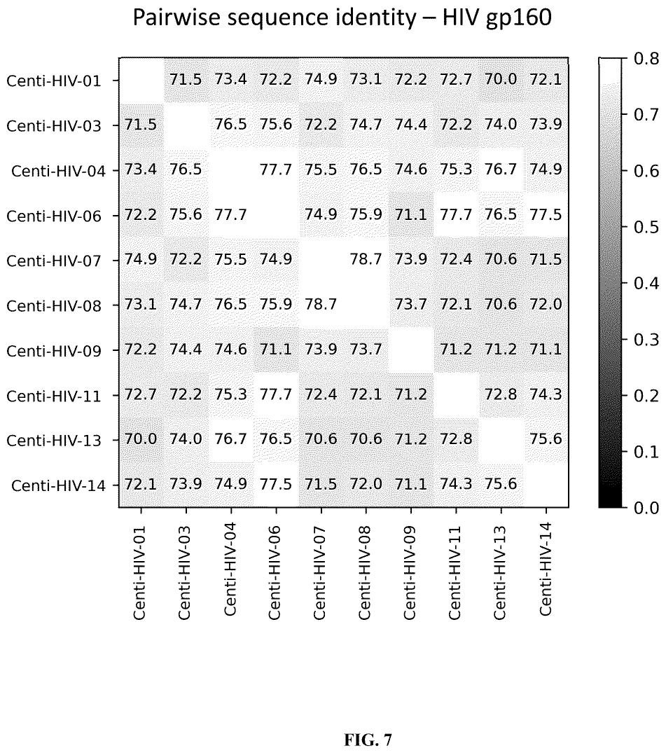

Each antigen was recombinantly expressed in HEK293 cells by Sino Biological using methods well-known in the art. Amino acid sequences for each immunogen are disclosed in SEQ ID NOs: 37, 41, 45, 49, 53, 57, 61, 65, 69, 73. Each immunogen comprises the given strain gp120, followed by a glycine-serine flexible linker (SEQ ID NO. 39), the given strain gp41, a 3C protease cleavage site, a fold-on domain to promote trimerization, and a C-terminal His-tag. Serum ELISA was performed as described in Example 1. FIG. 7 shows that any two of the ten homologous distinct antigen components in the Centi-HIV composition have pairwise sequence identity less than 80% and pairwise sequence identity greater than 70%.

### Example 4: Serum Reactivity to Venom Protein Antigens in Mice

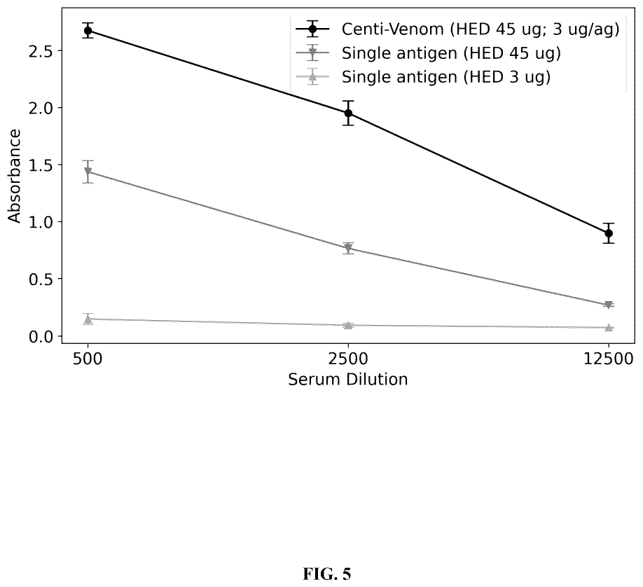

Centi-Venom is a mixture of snake venom toxin proteins from 15 distinct snake species (Agkistrodon contortrix contortrix, Agkistrodon piscivorus leucostoma, Agkistrodon piscivorus piscivorus, Agkistrodon bilineatus, Bitis arietans, Bothrops alternatus, Bothrops asper, Bothrops atrox, Bothrops jararaca, Crotalus atrox, Crotalus horridus, Daboia russelii, Daboia russelii limitis, Deinagkistrodon acutus, Echis ocellatus). Mice were immunized six times at two-week intervals with either Centi-Venom, or snake venom of a single snake species (Single antigen) (Crotalus horridus). Serum was extracted from blood drawn one week after the last immunization. Serum reactivity against the snake venom of the single snake species (Crotalus horridus) is shown in FIG. 5. FIG. 5 demonstrates that at a per-antigen human-equivalent (i.e., allometrically scaled) dose of 3 ug (=total human-equivalent dose (“HED”) of 45 ug), Centi-Venom results in a potent response while Single antigen at 3 ug exhibits no reactivity even against the same venom used for immunization. This cannot be explained by total antigen dose, as Single antigen at 45 ug still mounts a deficient response compared to Centi-Venom. Therefore, it is the unique combination of (1) using six or more homologous distinct antigen components and (2) each component being administered a low dose, that achieves the desired serological effect.

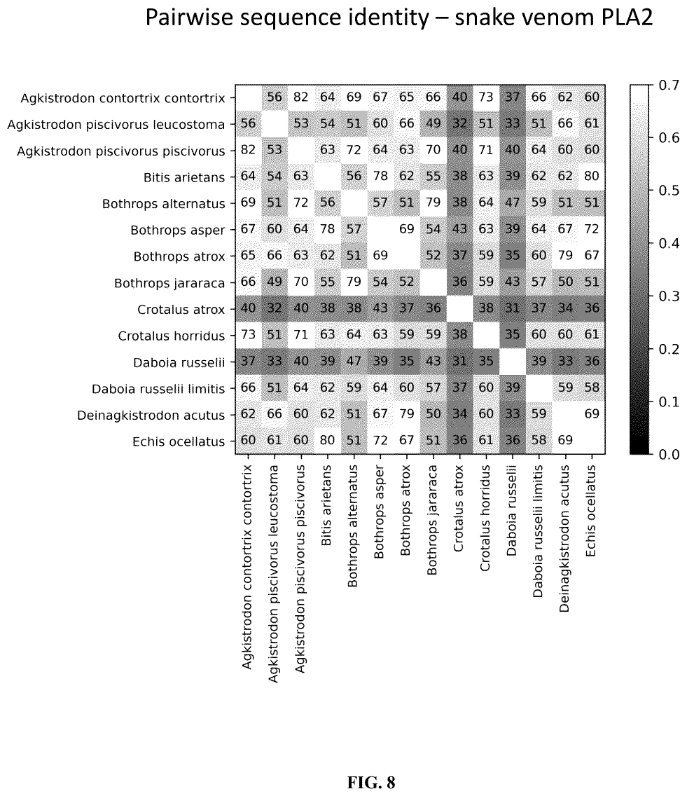

Serum ELISA was performed as described in Example 1. Amino acid sequences for the primary antigen (phospholipase A2; PLA2) in each snake venom are given in SEQ ID NOs 77-90. FIG. 8 shows that any two of the homologous distinct antigen components in the Centi-Venom composition have pairwise sequence identity less than 85% and pairwise sequence identity greater than 30%.

In some embodiments, the invention comprises a method of inducing an immune response against a pathogen in a subject comprising administering a vaccine composition of this invention. In some embodiments, the method is for prophylaxis against disease caused by the pathogen.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Certain Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise. In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “pharmaceutically acceptable” refers to approved or approvable by a regulatory agency of the Federal or a state government or listed in the U.S. Pharmacopeia (U.S.P.) or other generally recognized pharmacopeia for use in animals, including humans.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

1. A vaccine composition comprising:
(a) a plurality of RNAs that collectively encode for six or more homologous distinct antigen components, wherein any two of said six or more homologous distinct antigen components share less than 98% sequence identity, wherein each RNA of the plurality of RNAs that encodes for a distinct antigen component is present in the composition in an amount from 1 ng to 5 micrograms per dose;
(b) a plurality of viral-like particles (VLPs) that collectively display at least six or more homologous distinct antigen components, wherein any two of said six or more homologous antigen components share less than 98% sequence identity, wherein each distinct antigen component is present in the composition in an amount from 1 ng to 5 micrograms; or
(c) at least six or more homologous distinct antigen components, wherein any two of said six or more homologous antigen components share less than 98% sequence identity, wherein each distinct antigen component of said six or more homologous distinct antigen components is present in the composition in an amount from 550 ng to 5 micrograms.

2. (canceled)

3. The vaccine composition of claim 1, wherein each RNA that encodes for a distinct antigen component is present in the composition in an amount from 1 ng to 2.5 micrograms per dose.

4. (canceled)

5. The vaccine composition of claim 1, wherein each RNA that encodes for a distinct antigen component is present in the composition in an amount from 1 ng to 1 microgram per dose.

6.-13. (canceled)

14. The vaccine composition of claim 1, wherein each distinct antigen component displayed on VLPs is present in the composition in an amount from 1 ng to 2.5 micrograms per dose.

15. (canceled)

16. The vaccine composition of claim 1, wherein each distinct antigen component displayed on VLPs is present in the composition in an amount from 1 ng to 1 microgram per dose.

17.-20. (canceled)

21. The vaccine composition of claim 1, wherein each of the at least six or more homologous distinct antigen components is present in the composition in an amount from 550 ng to 3000 ng.

22.-25. (canceled)

26. The vaccine composition of claim 1, wherein said vaccine composition is for administration in a human subject.

27. The vaccine composition of claim 26, wherein said vaccine composition is for administration in an adult who is 18 years of age or older.

28.-33. (canceled)

34. The vaccine composition of claim 1, wherein said vaccine composition is for administration in an animal.

35.-39. (canceled)

40. The vaccine composition of claim 1, wherein the homologous distinct antigen components comprise a component of a virus.

41. (canceled)

42. The vaccine composition of claim 1, wherein at least two homologous distinct antigen components of said six or more homologous distinct antigen components comprise receptor binding domains for a cell surface protein, wherein the receptor binding domains share less than 99% sequence identity.

43.-52. (canceled)

53. The vaccine composition of claim 1, wherein an administration of said vaccine composition reduces a dominant population of immunogenic single variant epitopes corresponding to said homologous distinct antigen components.

54. The vaccine composition of claim 1, wherein any 2 of said six or more homologous distinct antigen components share less than about 97.5% sequence identity.

55.-64. (canceled)

65. The vaccine composition of claim 1, wherein said six or more homologous distinct antigen components comprise 7 or more homologous distinct antigen components.

66.-70. (canceled)

71. The vaccine composition of claim 1, wherein any 2 of said 6 or more homologous distinct antigen components share at least 30% sequence identity.

72.-75. (canceled)

76. The vaccine composition of claim 1, wherein said vaccine composition further comprises an adjuvant.

77.-80. (canceled)

81. A method of inducing an immune response against an influenza pathogen in a subject comprising administering the vaccine composition of claim 1.

82. The method of claim 81, where the method is for prophylaxis against influenza.

83. The vaccine composition of claim 1, wherein the six or more homologous distinct antigen components comprise an amino acid with at least 80% sequence identity to any one of SEQ ID NO: 3, 5, 7, 9, 11, 13, 15, 17, 19, 21, 23, 25, 27, 29, 31, 33, or 35.

84. The vaccine composition of claim 1, wherein the six or more homologous distinct antigen components comprise amino acid sequences having at least 80% sequence identity to each of SEQ ID NOs: 3, 5, 7, 9, 11, 13, 15, 17, 19, 21, 23, 25, 27, 29, 31, 33, or 35, respectively.
